SEQUENCE LISTING (17 sequences)

SEQ ID NO: 1 - protein, 21 aa, Artificial Sequence
  A-chain of analogue [A8H] of human insulin
GIVEQCCHSICSLYQLENYCN

SEQ ID NO: 2 - protein, 21 aa, Artificial Sequence
  A-chain of analogue [A8H, A21A] of human insulin
GIVEQCCHSICSLYQLENYCA

SEQ ID NO: 3 - protein, 21 aa, Artificial Sequence
  A-chain of analogue [A8H, A21G] of human insulin
GIVEQCCHSICSLYQLENYCG

SEQ ID NO: 4 - protein, 21 aa, Artificial Sequence
  A-chain of analogue [A14E] of human insulin
GIVEQCCTSICSLEQLENYCN

SEQ ID NO: 5 - protein, 21 aa, Artificial Sequence
  A-chain of analogue [A14E, A21A] of human insulin
GIVEQCCTSICSLEQLENYCA

SEQ ID NO: 6 - protein, 21 aa, Artificial Sequence
  A-chain of analogue [A21A] of human insulin
GIVEQCCTSICSLYQLENYCA

SEQ ID NO: 7 - protein, 21 aa, Artificial Sequence
  A-chain of analogue [A21G] of human insulin
GIVEQCCTSICSLYQLENYCG

SEQ ID NO: 8 - protein, 29 aa, Artificial Sequence
  B-chain of analogue [B3E, B26E, desB30] of human insulin
  MOD_RES (29)..(29): ACETYLATION
FVEQHLCGDHLVEALYLVCGERGFFETPK

SEQ ID NO: 9 - protein, 29 aa, Artificial Sequence
  B-chain of analogue [B3E, B26E, B28E, desB30] of human insulin
  MOD_RES (29)..(29): ACETYLATION
FVEQHLCGDHLVEALYLVCGERGFFETEK

SEQ ID NO: 10 - protein, 29 aa, Artificial Sequence
  B-chain of analogue [B3E, B27E, desB30] of human insulin
  MOD_RES (29)..(29): ACETYLATION
FVEQHLCGDHLVEALYLVCGERGFFYEPK

SEQ ID NO: 11 - protein, 29 aa, Artificial Sequence
  B-chain of analogue [B3E, B27E, B28D, desB30] of human insulin
  MOD_RES (29)..(29): ACETYLATION
FVEQHLCGDHLVEALYLVCGERGFFYEDK

SEQ ID NO: 12 - protein, 29 aa, Artificial Sequence
  B-chain of analogue [B3E, B27E, B28E, desB30] of human insulin
  MOD_RES (29)..(29): ACETYLATION
FVEQHLCGDHLVEALYLVCGERGFFYEEK

SEQ ID NO: 13 - protein, 29 aa, Artificial Sequence
  B-chain of analogue [B3E, B28D, desB30] of human insulin
  MOD_RES (29)..(29): ACETYLATION
FVEQHLCGDHLVEALYLVCGERGFFYTDK

SEQ ID NO: 14 - protein, 29 aa, Artificial Sequence
  B-chain of analogue [B3E, B28E, desB30] of human insulin
  MOD_RES (29)..(29): ACETYLATION
FVEQHLCGDHLVEALYLVCGERGFFYTEK

SEQ ID NO: 15 - protein, 29 aa, Artificial Sequence
  B-chain of analogue [B3Q, B26E, desB30] of human insulin
  MOD_RES (29)..(29): ACETYLATION
FVQQHLCGDHLVEALYLVCGERGFFETPK

SEQ ID NO: 16 - protein, 29 aa, Artificial Sequence
  B-chain of analogue [B3Q, B28D, desB30] of human insulin
  MOD_RES (29)..(29): ACETYLATION
FVQQHLCGDHLVEALYLVCGERGFFYTDK

SEQ ID NO: 17 - protein, 29 aa, Artificial Sequence
  B-chain of analogue [B3Q, B28E, desB30] of human insulin
  MOD_RES (29)..(29): ACETYLATION
FVQQHLCGDHLVEALYLVCGERGFFYTEK

(19) United States
(12) Patent Application Publication
(10) Pub. No.: US 2019/0010206 A1
(43) Pub. Date: Jan. 10, 2019

(54) Novel Insulin Derivatives and the Medical Uses Hereof

(71) Applicant: Novo Nordisk A/S, Bagsvaerd (DK)

(72) Inventors: Peter Madsen, Bagsvaerd (DK); Martin Muenzel, Broenshoej (DK); Claudia Ulrich Hjoerringgaard, Glostrup (DK); Susanne Hostrup, Vaerloese (DK); Tine Glendorf, Hilleroed (DK); Mathias Norrman, Staffanstorp (SE); Christian Fledelius, Copenhagen O (DK)

(21) Appl. No.: 15/754,173

(22) PCT Filed: Aug. 24, 2016
(86) PCT No.: PCT/EP2016/069969

§ 371 Date: Feb. 21, 2018

(30) Foreign Application Priority Data
Aug. 25, 2015 (EP) 15182279.8

(51) Int. Cl. C07K 14/62; A61K 38/28; A61K 31/4406; A61P 3/10

(52) U.S. Cl. CPC C07K 14/62; A61P 3/10; A61K 31/4406; A61K 38/28

(57) ABSTRACT

The present invention is in the therapeutic fields of drugs for medical conditions relating to diabetes. More specifically the invention relates to novel acylated derivatives of human insulin analogues. The invention also provides pharmaceutical compositions comprising such insulin derivatives, and relates to the use of such derivatives for the treatment or prevention of medical conditions relating to diabetes.

### TECHNICAL FIELD

The present invention is in the therapeutic fields of drugs for medical conditions relating to diabetes. More specifically the invention relates to novel acylated derivatives of human insulin analogues. The invention also provides pharmaceutical compositions comprising such derivatized insulin analogues, and relates to the use of such derivatives for the treatment or prevention of medical conditions relating to diabetes.

### BACKGROUND OF THE INVENTION

Insulin therapy for the treatment of diabetes has been used for decades. Insulin therapy usually involves administering several injections of insulin each day. Such therapy usually involves administration of a long-acting basal injection once or twice daily, and an injection of a fast-acting insulin at mealtime (i.e. prandial use). One of the key improvements in insulin therapy was the introduction of rapid-acting insulin analogues. However, even with the rapid-acting insulin analogues, peak insulin levels typically do not occur until 50 to 70 minutes following the injection.

Therefore insulin injections do not replicate the natural time-action profile of insulin. In particular, the natural spike of the first-phase insulin release in a person without diabetes results in blood insulin levels rising within several minutes of the entry into the blood of glucose from a meal. By contrast, injected insulin enters the blood only slowly, with peak insulin levels occurring within 80 to 100 minutes following the injection of regular human insulin.

Because the rapid-acting insulin analogues do not adequately mimic the first-phase insulin release, diabetics using insulin therapy continue to have inadequate levels of insulin present at the initiation of a meal, and too much insulin present between meals. This lag in insulin delivery can result in hyperglycemia early after meal onset.

Insulin possesses self-association properties, and its concentration represents a major factor of self-association. At high concentrations, especially in pharmaceutical formulations, insulin will self-associate into dimer, hexamer, dodecamer, and crystal. However, the physiologically active form of insulin is the monomer, which binds with the insulin receptor and triggers a biological response.

The rapidity of insulin action is dependent on how quickly the insulin is absorbed from the subcutaneous tissue. When regular human insulin is injected subcutaneously, the formulation is primarily composed of hexamers containing two zinc ions. Due to its size, the hexameric insulin has a lower rate of diffusion and consequently, the absorption rate is slower than for smaller species.

Located within the hexamer are two zinc atoms that stabilize the molecule towards chemical and physical degradation. Post injection, a concentration driven dynamic equilibrium occurs in the subcutaneous tissue, causing the hexamers to dissociate into dimers, and then to monomers. Historically, these regular human insulin formulations require approximately 120 minutes to reach maximum plasma concentration levels. Zinc-insulin preparations, that are more quickly absorbed than regular human insulin, have been commercialised, e.g. insulin aspart and insulin lispro.

Zinc-free insulin formulations would enable faster subcutaneous absorption, but for insulins in general, the chemical and physical stability of zinc-free formulations is a challenge.

Various insulin derivatives have been suggested for different formulations and uses:

WO 1998 042749 describes zinc-free insulin crystals for pulmonary administration, WO 2002 076495 describes zinc-free and low-zinc insulin preparations having improved stability, and WO 2013 063572 describes ultra-concentrated rapid-acting insulin analogue formulations optionally devoid of zinc.

Finally, WO 9731022, WO 2005 012347, WO 2006 125765 and WO 2009 02206 describe certain acylated insulins.

Moreover, acylation of peptides and proteins with albumin binding moieties have been used to prolong the duration of action of the peptides and proteins.

However, the insulin derivatives according to the present invention have not been reported, and their use as fast acting insulin derivatives for prandial use has never been suggested.

### OBJECTS OF THE INVENTION

It is an object of the invention to provide insulin analogues that have a prandial profile following subcutaneous administration.

Another object of the invention is to provide insulin analogues that are chemically stable in formulation.

A third object of the invention is to provide insulin analogues that are chemically stable in formulation without added zinc.

A fourth object of the invention is to provide insulin analogues that are physically stable in formulation.

A fifth object of the invention is to provide insulin analogues that are physically stable in formulation without added zinc.

A sixth object of the invention is to provide insulin analogues that are chemically and physically stable in formulation.

A seventh object of the invention is to provide insulin analogues that are chemically and physically stable in formulation without added zinc.

An eight object of the invention is to provide insulin analogues that are hepatopreferential relative to currently marketed prandial insulins following subcutaneous administration.

A ninth object of the invention is to provide insulin analogues that are hepatoselective relative to currently marketed prandial insulins following subcutaneous administration.

A tenth object of the invention is to provide insulin analogues that are less prone to induce hypoglycaemia relative to currently marketed prandial insulins following prandial subcutaneous administration.

An eleventh object of the invention is to provide insulin analogues that are less prone to induce weight gain relative to currently marketed prandial insulins following prandial subcutaneous administration.

A twelfth object of the invention is to provide insulin analogues that are less prone to induce hypoglycaemia and weight gain relative to currently marketed prandial insulins following prandial subcutaneous administration.

A thirteenth object of the invention is to provide insulin analogues that have less action in muscle and or fat tissue relative to currently marketed prandial insulins following subcutaneous administration.

Further objects of this invention are drawn to combinations of one or more of the objects mentioned above, and in particular the provision of insulin analogues that show a prandial profile following subcutaneous administration, while being chemically stable in formulations, and in particular in formulations without added zinc.

### SUMMARY OF THE INVENTION

We have discovered that the acylated insulin derivatives of the present invention have significantly improved properties relative to similar insulin derivatives of the prior art. We have in particular discovered that the insulin derivatives of the invention, in formulations containing no added zinc ions, and when compared to similar derivatives of the prior art, are associated with a smaller size of the molecular aggregates. Smaller species are known to diffuse more rapidly than larger species, and faster absorption is consequently to be expected. The size of these molecular aggregates can e.g. be measured as described herein by Small Angle X-ray Scattering (SAXS) as described in the examples section.

We have also discovered that the insulin derivatives of the invention, relative to similar derivatives of the prior art, in formulations containing no added zinc ions, are absorbed more rapidly after subcutaneous administration to pigs and/or rats, thereby demonstrating a potential clinical utility as insulins for prandial use. We have discovered that the insulin derivatives of the invention, relative to similar derivatives of the prior art, in formulations containing no added zinc ions are associated with less “tailing” following subcutaneous administration to pigs. By less tailing is meant that the subcutaneous depot of injected insulin is absorbed more rapidly than for similar analogues of the prior art, so that the mean residence time (MRT) following subcutaneous administration is shorter for the insulin derivatives of the invention when compared to similar acylated derivatives of the prior art.

Zinc-free formulations enable faster subcutaneous absorption, but for insulins in general, chemical and physical stability of zinc-free formulations is a challenge, and has until now only been shown to be possible with insulin glulisine (Apidra®; B3K, B29E human insulin), and only in the presence of surfactants when dispensed in vials.

We have now discovered that the acylated insulin derivatives of the invention, with substitutions in position B3, very unexpectedly and unprecedented are both chemically and physically stable in formulations with no added zinc-ions and no added surfactants.

The rate of absorption of insulin following subcutaneous administration is to a large extent correlated by the rate of diffusion. Thus, smaller species have faster diffusion rates and show faster rates of absorption when compared to larger species.

Insulin preparations containing zinc are absorbed more slowly than zinc-free formulations as the zinc-hexamers of the formulation needs to dissociate to dimers and/or monomers before absorption can take place.

Chemical and physical stability of insulin formulations requires presence of zinc, and absence of zinc is required for fast absorption. A solution to this problem is provided in the present invention.

Since insulin needs to be stable in formulation in order to be clinically useful, the property of the insulins of the invention being stable in zinc-free formulation results in pharmacokinetic and pharmacodynamic properties superior to those of the insulins of the prior art. This is because that the insulins of the prior art need to be formulated with zinc ions in order to be stable in formulation. The proper comparison regarding pharmacokinetic and pharmacodynamic properties is thus to compare stable formulations and, consequently, to compare stable zinc-free formulations of insulins of the invention with zinc-containing formulations of insulins of the prior art.

An advantage of using acylated insulin derivatives as prandial insulin therapy is to achieve higher plasma insulin concentrations than those achieved by treatment with un-acylated prandial insulins, like insulin aspart, insulin lispro or insulin glulisine.

The acylated insulin derivatives according to the invention have a prandial-like time-action profile following subcutaneous administration.

The acylated insulin derivatives with tetradecanedioic acid, pentadecanedioic acid, or hexadecanedioic acid based albumin binders according to the invention have shown to confer high insulin receptor binding affinities, affinities that are reduced in the presence of 1.5% human serum albumin (HSA).

The acylated insulin derivatives according to the invention do not have reduced solubility at physiological salt concentrations.

Accordingly, in its first aspect, the invention provides novel insulin derivatives, which insulin derivatives are acylated derivatives of human insulin analogues, which analogues are [B3aar¹, desB30] relative to human insulin; wherein

aar¹ represents Glu (E), Gln (Q), Asp (D), Ser (S) or Thr (T); and

one or two of the amino acid residues located in positions B26, B27 and/or B28 are substituted for Glu (E) and/or Asp (D);

which analogue may additionally comprise an A8aar² substitution, and/or an A14Glu (E) substitution, and/or an A21aar³ substitution; wherein

aar² represents His (H) or Arg (R); and

aar³ represents Gly (G) or Ala (A);

which insulin analogue is derivatized by acylation of the epsilon amino group of the naturally occurring lysine residue at the B29 position with a group of Formula II

[Acyl]-[Linker]-

wherein the Linker group is an amino acid chain composed of from 1 to 10 amino acid residues selected from gGlu and/or OEG; wherein

gGlu represents a gamma glutamic acid residue;

OEG represents a residue of 8-amino-3,6-dioxaoctanoic acid (i.e. a group of the formula —NH—(CH₂)₂—O—(CH₂)₂—O—CH₂—CO—);

which amino acid residues may be present in any order; and

which amino acid chain comprises at least one gGlu residue; and

wherein the Acyl group is a residue of an α,ω-di-carboxylic acid selected from 1,14-tetradecanedioic acid; 1,15-pentadecanedioic acid; and 1,16-hexadecanedioic acid.

In another first aspect, the invention provides pharmaceutical compositions comprising the insulin derivative of the invention, and one or more pharmaceutically acceptable excipients.

In a further aspect, the invention relates to use of the insulin derivative of the invention as a medicament.

In a yet further aspect the invention provides methods for the treatment, prevention or alleviation of diseases, disorders or conditions relating to diabetes, Type 1 diabetes, Type 2 diabetes, impaired glucose tolerance, hyperglycemia, dyslipidemia, obesity, metabolic syndrome (metabolic syndrome X, insulin resistance syndrome), hypertension, cognitive disorders, atherosclerosis, myocardial infarction, stroke, cardiovascular disorders, coronary heart disease, inflammatory bowel syndrome, dyspepsia, or gastric ulcers, which method comprises administration to a subject in need thereof a therapeutically effective amount of the insulin derivative of the invention.

Other objects of the invention will be apparent to the person skilled in the art from the following detailed description and examples.

### DETAILED DESCRIPTION OF THE INVENTION

### Insulin Derivatives

In its first aspect the present invention provides novel insulin derivatives, which insulin derivative are acylated analogues of human insulin.

The insulin derivative of the invention may in particular be characterised as an acylated analogue of human insulin, which analogue is [B3aar¹, desB30] relative to human insulin; wherein

aar¹ represents Glu (E), Gln (Q), Asp (D), Ser (S) or Thr (T); and

one or two of the amino acid residues located in positions B26, B27 and/or B28 are substituted for Glu (E) and/or Asp (D);

which analogue may additionally comprise an A8aar² substitution, and/or an A14Glu (E) substitution, and/or an A21aar³ substitution; wherein

aar² represents His (H) or Arg (R); and

aar³ represents Gly (G) or Ala (A);

which insulin analogue is derivatized by acylation of the epsilon amino group of the naturally occurring lysine residue at the B29 position with a group of Formula II

[Acyl]-[Linker]-

wherein the Linker group is an amino acid chain composed of from 1 to 10 amino acid residues selected from gGlu and/or OEG; wherein

gGlu represents a gamma glutamic acid residue;

OEG represents a residue of 8-amino-3,6-dioxaoctanoic acid (i.e. a group of the formula —NH—(CH₂)₂—O—(CH₂)₂—O—CH₂—CO—);

which amino acid residues may be present in any order; and

which amino acid chain comprises at least one gGlu residue; and

wherein the Acyl group is a residue of an α,ω-di-carboxylic acid selected from 1,14-tetradecanedioic acid; 1,15-pentadecanedioic acid; and 1,16-hexadecanedioic acid.

### Preferred Features of the Invention

The acylated analogue of human insulin of the invention may be further characterised by reference to one or more of the following clauses:

1. An acylated analogue of human insulin, which analogue is [B3aar¹, desB30] relative to human insulin; wherein aar¹ represents Glu (E), Gln (Q), Asp (D), Ser (S) or Thr (T).

2. An acylated analogue of human insulin, which analogue is [B3aar¹, desB30] relative to human insulin; wherein aar¹ represents Glu (E) or Gln (Q).

3. An acylated analogue of human insulin, which analogue is [B3aar¹, B26aar⁴, desB30] relative to human insulin; wherein aar¹ represents Glu (E), Gln (Q), Asp (D), Ser (S) or Thr (T); and aar⁴ represents Glu (E) and/or Asp (D).

4. An acylated analogue of human insulin, which analogue is [B3aar¹, B26aar⁴, desB30] relative to human insulin, wherein aar¹ represents Glu (E) or Gln (Q); and aar⁴ represents Glu (E).

5. An acylated analogue of human insulin, which analogue is [B3aar¹, B26aar⁴, desB30] relative to human insulin, wherein aar¹ represents Glu (E); and aar⁴ represents Glu (E).

6. The acylated analogue of clause 3, wherein [B3aar¹, B26aar⁴, desB30] analogue of the invention is

B3E, B26E, B29K(N(eps)tetradecanedioyl-4×gGlu), desB30 human insulin;

B3E, B26E, B29K(N(eps)tetradecanedioyl-gGlu-2×OEG), desB30 human insulin; or

B3Q, B26E, B29K(N(eps)tetradecanedioyl-4×gGlu), desB30 human insulin.

7. An acylated analogue of human insulin, which analogue is [B3aar¹, B27aar⁴, desB30] relative to human insulin; wherein aar¹ represents Glu (E), Gln (Q), Asp (D), Ser (S) or Thr (T); and aar⁴ represents Glu (E) and/or Asp (D).

8. An acylated analogue of human insulin, which analogue is [B3aar¹, B28aar⁴, desB30] relative to human insulin; wherein aar¹ represents Glu (E), Gln (Q), Asp (D), Ser (S) or Thr (T); and aar⁴ represents Glu (E) and/or Asp (D).

9. An acylated analogue of human insulin, which analogue is [B3aar¹, B28aar⁴, desB30] relative to human insulin; wherein aar¹ represents Glu (E) or Gln (Q); and aar⁴ represents Glu (E) or Asp (D).

10. The acylated analogue of clause 8, wherein the [B3aar¹, B28aar⁴, desB30] analogue of the invention is

B3E, B28D, B29K(N(eps)tetradecanedioyl-4×gGlu), desB30 human insulin;

B3E, B28D, B29K(N(eps)tetradecanedioyl-gGlu-2×OEG), desB30 human insulin;

B3E, B28E, B29K(N(eps)tetradecanedioyl-4×gGlu), desB30 human insulin;

B3E, B28E, B29K(N(eps)tetradecanedioyl-gGlu-2×OEG), desB30 human insulin;

B3Q, B28D, B29K(N(eps)tetradecanedioyl-4×gGlu), desB30 human insulin;

B3Q, B28D, B29K(N(eps)tetradecanedioyl-gGlu-2×OEG), desB30 human insulin; or

B3Q, B28E, B29K(N(eps)tetradecanedioyl-4×gGlu), desB30 human insulin.

11. An acylated analogue of human insulin, which analogue is [B3aar¹, B26aar⁴, B27aar⁴, desB30] relative to human insulin; wherein aar¹ represents Glu (E), Gln (Q), Asp (D), Ser (S) or Thr (T); and aar⁴ independently of each other represent Glu (E) and/or Asp (D).

12. An acylated analogue of human insulin, which analogue is [B3aar¹, B26aar⁴, B28aar⁴, desB30] relative to human insulin; wherein aar¹ represents Glu (E), Gln (Q), Asp (D), Ser (S) or Thr (T); and aar⁴ independently of each other represent Glu (E) and/or Asp (D).

13. An acylated analogue of human insulin, which analogue is [B3aar¹, B26aar⁴, B28aar⁴, desB30] relative to human insulin; wherein aar¹ represents Glu (E); and both of aar⁴ represent Glu (E).

14. The acylated analogue of clause 12, wherein the [B3aar¹, B26aar⁴, B28aar⁴, desB30] analogue of the invention is

B3E, B26E, B28E, B29K(N(eps)tetradecanedioyl-4×gGlu), desB30 human insulin; or

B3E, B26E, B28E, B29K(N(eps)tetradecanedioyl-gGlu-2×OEG), desB30 human insulin.

15. An acylated analogue of human insulin, which analogue is [B3aar¹, B27aar⁴, B28aar⁴, desB30] relative to human insulin; wherein aar¹ represents Glu (E), Gln (Q), Asp (D), Ser (S) or Thr (T); and aar⁴ independently of each other represent Glu (E) and/or Asp (D).

16. An acylated analogue of human insulin, which analogue is [B3aar¹, B27aar⁴, B28aar⁴, desB30] relative to human insulin; wherein aar¹ represents Glu (E); and aar⁴ represents Glu (E).

17. The acylated analogue of clause 15, wherein the [B3aar¹, B27aar⁴, B28aar⁴, desB30] analogue of the invention is

B3E, B27E, B28E, B29K(N(eps)tetradecanedioyl-gGlu-2×OEG), desB30 human insulin; or

B3E, B27E, B28E, B29K(N(eps)tetradecanedioyl-4×gGlu), desB30 human insulin.

18. An acylated analogue of human insulin, which analogue is [A8aar², B3aar¹, B26aar⁴, desB30] relative to human insulin; wherein aar¹ represents Glu (E), Gln (Q), Asp (D), Ser (S) or Thr (T); aar² represents His (H) or Arg (R); and aar⁴ represents Glu (E) and/or Asp (D).

19. An acylated analogue of human insulin, which analogue is [A8aar², B3aar¹, B27aar⁴, desB30] relative to human insulin; wherein aar¹ represents Glu (E), Gln (Q), Asp (D), Ser (S) or Thr (T); aar² represents His (H) or Arg (R); and aar⁴ represents Glu (E) and/or Asp (D).

20. An acylated analogue of human insulin, which analogue is [A8aar², B3aar¹, B28aar⁴, desB30] relative to human insulin; wherein aar¹ represents Glu (E), Gln (Q), Asp (D), Ser (S) or Thr (T); aar² represents His (H) or Arg (R); and aar⁴ represents Glu (E) and/or Asp (D).

21. An acylated analogue of human insulin, which analogue is [A8aar², B3aar¹, B28aar⁴, desB30] relative to human insulin; wherein aar¹ represents Glu (E); aar² represents His (H); and aar⁴ represents Asp (D).

22. The acylated analogue of clause 20, wherein the [A8aar², B3aar¹, B28aar⁴, desB30] analogue of the invention is

A8H, B3E, B28D, B29K(N(eps)tetradecanedioyl-gGlu-2×OEG), desB30 human insulin; or

A8H, B3E, B28D, B29K(N(eps)tetradecanedioyl-4×gGlu), desB30 human insulin.

23. An acylated analogue of human insulin, which analogue is [A8aar², B3aar¹, B26aar⁴, B27aar⁴, desB30] relative to human insulin; wherein aar¹ represents Glu (E), Gln (Q), Asp (D), Ser (S) or Thr (T); aar² represents His (H) or Arg (R); and aar⁴ independently of each other represent Glu (E) and/or Asp (D).

24. An acylated analogue of human insulin, which analogue is [A8aar², B3aar¹, B26aar⁴, B28aar⁴, desB30] relative to human insulin; wherein aar¹ represents Glu (E), Gln (Q), Asp (D), Ser (S) or Thr (T); aar² represents His (H) or Arg (R); and aar⁴ independently of each other represent Glu (E) and/or Asp (D).

25. An acylated analogue of human insulin, which analogue is [A8aar², B3aar¹, B27aar⁴, B28aar⁴, desB30] relative to human insulin; wherein aar¹ represents Glu (E), Gln (Q), Asp (D), Ser (S) or Thr (T); aar² represents His (H) or Arg (R); and aar⁴ independently of each other represent Glu (E) and/or Asp (D).

26. An acylated analogue of human insulin, which analogue is [A8aar², B3aar¹, B27aar⁴, B28aar⁴, desB30] relative to human insulin; wherein aar¹ represents Glu (E); aar² represents His (H); and aar⁴ represents Glu (E).

27. The acylated analogue of clause 25, wherein the [A8aar², B3aar¹, B27aar⁴, B28aar⁴, desB30] analogue of the invention is

A8H, B3E, B27E, B28E, B29K(N(eps)tetradecanedioyl-gGlu-2×OEG), desB30 human insulin.

28. An acylated analogue of human insulin, which analogue is [A14Glu, B3aar¹, B26aar⁴, desB30] relative to human insulin; wherein aar¹ represents Glu (E), Gln (Q), Asp (D), Ser (S) or Thr (T); and aar⁴ represents Glu (E) and/or Asp (D).

29. An acylated analogue of human insulin, which analogue is [A14Glu, B3aar¹, B27aar⁴, desB30] relative to human insulin; wherein aar¹ represents Glu (E), Gln (Q), Asp (D), Ser (S) or Thr (T); and aar⁴ represents Glu (E) and/or Asp (D).

30. An acylated analogue of human insulin, which analogue is [A14Glu, B3aar¹, B28aar⁴, desB30] relative to human insulin; wherein aar¹ represents Glu (E), Gln (Q), Asp (D), Ser (S) or Thr (T); and aar⁴ represents Glu (E) and/or Asp (D).

31. An acylated analogue of human insulin, which analogue is [A14Glu, B3aar¹, B28aar⁴, desB30] relative to human insulin; wherein aar¹ represents Gln (Q); and aar⁴ represents Asp (D).

32. The acylated analogue of clause 30, wherein the [A14Glu, B3aar¹, B28aar⁴, desB30] analogue of the invention is

A14E, B3Q, B28D, B29K(N(eps)tetradecanedioyl-4×gGlu), desB30 human insulin.

33. An acylated analogue of human insulin, which analogue is [A14Glu, B3aar¹, B26aar⁴, B27aar⁴, desB30] relative to human insulin; wherein aar¹ represents Glu (E), Gln (Q), Asp (D), Ser (S) or Thr (T); and aar⁴ independently of each other represent Glu (E) and/or Asp (D).

34. An acylated analogue of human insulin, which analogue is [A14Glu, B3aar¹, B26aar⁴, B28aar⁴, desB30] relative to human insulin; wherein aar¹ represents Glu (E), Gln (Q), Asp (D), Ser (S) or Thr (T); and aar⁴ independently of each other represent Glu (E) and/or Asp (D).

35. An acylated analogue of human insulin, which analogue is [A14Glu, B3aar¹, B27aar⁴, B28aar⁴, desB30] relative to human insulin; wherein aar¹ represents Glu (E), Gln (Q), Asp (D), Ser (S) or Thr (T); and aar⁴ independently of each other represent Glu (E) and/or Asp (D).

36. An acylated analogue of human insulin, which analogue is [A21aar³, B3aar¹, B26aar⁴, desB30] relative to human insulin; wherein aar¹ represents Glu (E), Gln (Q), Asp (D), Ser (S) or Thr (T); aar³ represents Gly (G) or Ala (A); and aar⁴ represents Glu (E) and/or Asp (D).

37. An acylated analogue of human insulin, which analogue is [A21aar³, B3aar¹, B26aar⁴, desB30] relative to human insulin; wherein aar¹ represents Glu (E) or Gln (Q); aar³ represents Gly (G) or Ala (A); and aar⁴ represents Glu (E).

38. An acylated analogue of human insulin, which analogue is [A21aar³, B3aar¹, B26aar⁴, desB30] relative to human insulin; wherein aar¹ represents Glu (E); aar³ represents Gly (G) or Ala (A); and aar⁴ represents Glu (E).

39. The acylated analogue of clause 36, wherein the [A21aar³, B3aar¹, B26aar⁴, desB30] analogue of the invention is

A21A, B3E, B26E, B29K(N(eps)tetradecanedioyl-4×gGlu), desB30 human insulin;

A21A, B3E, B26E, B29K(N(eps)tetradecanedioyl-gGlu-2×OEG), desB30 human insulin;

A21G, B3E, B26E, B29K(N(eps)tetradecanedioyl-gGlu-2×OEG), desB30 human insulin;

A21G, B3E, B26E, B29K(N(eps)tetradecanedioyl-4×gGluG), desB30 human insulin; or

A21A, B3Q, B26E, B29K(N(eps)tetradecanedioyl-4×gGlu), desB30 human insulin.

40. An acylated analogue of human insulin, which analogue is [A21aar³, B3aar¹, B27aar⁴, desB30] relative to human insulin; wherein aar¹ represents Glu (E), Gln (Q), Asp (D), Ser (S) or Thr (T); aar³ represents Gly (G) or Ala (A); and aar⁴ represents Glu (E) and/or Asp (D).

41. An acylated analogue of human insulin, which analogue is [A21aar³, B3aar¹, B27aar⁴, desB30] relative to human insulin; wherein aar¹ represents Glu (E); aar³ represents Gly (G); and aar⁴ represents Glu (E).

42. The acylated analogue of clause 40, wherein the [A21aar³, B3aar¹, B27aar⁴, desB30] analogue of the invention is

A21G, B3E, B27E, B29K(N(eps)tetradecanedioyl-4×gGlu), desB30 human insulin.

43. An acylated analogue of human insulin, which analogue is [A21aar³, B3aar¹, B28aar⁴, desB30] relative to human insulin; wherein aar¹ represents Glu (E), Gln (Q), Asp (D), Ser (S) or Thr (T); aar³ represents Gly (G) or Ala (A); and aar⁴ represents Glu (E) and/or Asp (D).

44. An acylated analogue of human insulin, which analogue is [A21aar³, B3aar¹, B28aar⁴, desB30] relative to human insulin; wherein aar¹ represents Glu (E) or Gln (Q); aar³ represents Gly (G) or Ala (A); and aar⁴ represents Glu (E) or Asp (D).

45. The acylated analogue of clause 43, wherein the [A21aar³, B3aar¹, B28aar⁴, desB30] analogue of the invention is

A21A, B3E, B28D, B29K(N(eps)tetradecanedioyl-4×gGlu), desB30 human insulin;

A21A, B3E, B28D, B29K(N(eps)tetradecanedioyl-gGlu-2×OEG), desB30 human insulin;

A21A, B3E, B28E, B29K(N(eps)tetradecanedioyl-4×gGlu), desB30 human insulin;

A21A, B3E, B28E, B29K(N(eps)tetradecanedioyl-gGlu-2×OEG), desB30 human insulin;

A21A, B3Q, B28D, B29K(N(eps)tetradecanedioyl-gGlu-2×OEG), desB30 human insulin;

A21G, B3E, B28D, B29K(N(eps)tetradecanedioyl-4×gGlu), desB30 human insulin;

A21G, B3E, B28D, B29K(N(eps)tetradecanedioyl-gGlu-2×OEG), desB30 human insulin;

A21G, B3E, B28D, B29K(N(eps)hexadecanedioyl-gGlu-2×OEG), desB30 human insulin;

A21G, B3E, B28D, B29K(N(eps)hexadecanedioyl-4×gGlu), desB30 human insulin;

A21G, B3E, B28E, B29K(N(eps)tetradecanedioyl-4×gGlu), desB30 human insulin;

A21G, B3E, B28E, B29K(N(eps)tetradecanedioyl-gGlu-2×OEG), desB30 human insulin;

A21A, B3Q, B28E, B29K(N(eps)tetradecanedioyl-4×gGlu), desB30 human insulin;

A21A, B3E, B28E, B29K(N(eps)hexadecanedioyl-4×gGlu), desB30 human insulin; or

A21A, B3E, B28E, B29K(N(eps)hexadecanedioyl-gGlu-2×OEG), desB30 human insulin.

46. An acylated analogue of human insulin, which analogue is [A21aar³, B3aar¹, B26aar⁴, B27aar⁴, desB30] relative to human insulin; wherein aar¹ represents Glu (E), Gln (Q), Asp (D), Ser (S) or Thr (T); aar³ represents Gly (G) or Ala (A); and aar⁴ independently of each other represent Glu (E) and/or Asp (D).

47. An acylated analogue of human insulin, which analogue is [A21aar³, B3aar¹, B26aar⁴, B28aar⁴, desB30] relative to human insulin; wherein aar¹ represents Glu (E), Gln (Q), Asp (D), Ser (S) or Thr (T); aar³ represents Gly (G) or Ala (A); and aar⁴ independently of each other represent Glu (E) and/or Asp (D).

48. An acylated analogue of human insulin, which analogue is [A21aar³, B3aar¹, B26aar⁴, B28aar⁴, desB30] relative to human insulin; wherein aar¹ represents Glu (E); aar³ represents Gly (G) or Ala (A); and aar⁴ both represent Glu (E).

49. The acylated analogue of clause 47, wherein the [A21aar³, B3aar¹, B26aar⁴, B28aar⁴, desB30] analogue of the invention is

A21A, B3E, B26E, B28E, B29K(N(eps)tetradecanedioyl-4×gGlu), desB30 human insulin;

A21A, B3E, B26E, B28E, B29K(N(eps)tetradecanedioyl-gGlu-2×OEG), desB30 human insulin;

A21G, B3E, B26E, B28E, B29K(N(eps)tetradecanedioyl-4×gGlu), desB30 human insulin; or

A21G, B3E, B26E, B28E, B29K(N(eps)tetradecanedioyl-gGlu-2×OEG), desB30 human insulin.

50. An acylated analogue of human insulin, which analogue is [A21aar³, B3aar¹, B27aar⁴, B28aar⁴, desB30] relative to human insulin; wherein aar¹ represents Glu (E), Gln (Q), Asp (D), Ser (S) or Thr (T); aar³ represents Gly (G) or Ala (A); and aar⁴ independently of each other represent Glu (E) and/or Asp (D).

51. An acylated analogue of human insulin, which analogue is [A21aar³, B3aar¹, B27aar⁴, B28aar⁴, desB30] relative to human insulin; wherein aar¹ represents Glu (E); aar³ represents Gly (G) or Ala (A); and aar⁴ independently of each other represent Glu (E) and/or Asp (D).

52. The acylated analogue of clause 50, wherein the [A21aar³, B3aar¹, B27aar⁴, B28aar⁴, desB30] analogue of the invention is

A21A, B3E, B27E, B28E, B29K(N(eps)tetradecanedioyl-4×gGlu), desB30 human insulin;

A21A, B3E, B27E, B28E, B29K(N(eps)tetradecanedioyl-gGlu-2×OEG), desB30 human insulin;

A21A, B3E, B27E, B28E, B29K(N(eps)hexadecanedioyl-4×gGlu), desB30 human insulin;

A21G, B3E, B27E, B28D, B29K(N(eps)tetradecanedioyl-gGlu-2×OEG), desB30 human insulin; or

A21G, B3E, B27E, B28E, B29K(N(eps)tetradecanedioyl-gGlu-2×OEG), desB30 human insulin.

53. An acylated analogue of human insulin, which analogue is [A8aar², A14Glu, B3aar¹, B26aar⁴, desB30] relative to human insulin; wherein aar¹ represents Glu (E), Gln (Q), Asp (D), Ser (S) or Thr (T); aar² represents His (H) or Arg (R); and aar⁴ represents Glu (E) and/or Asp (D).

54. An acylated analogue of human insulin, which analogue is [A8aar², A14Glu, B3aar¹, B27aar⁴, desB30] relative to human insulin; wherein aar¹ represents Glu (E), Gln (Q), Asp (D), Ser (S) or Thr (T); aar² represents His (H) or Arg (R); and aar⁴ represents Glu (E) and/or Asp (D).

55. An acylated analogue of human insulin, which analogue is [A8aar², A14Glu, B3aar¹, B28aar⁴, desB30] relative to human insulin; wherein aar¹ represents Glu (E), Gln (Q), Asp (D), Ser (S) or Thr (T); aar² represents His (H) or Arg (R); and aar⁴ represents Glu (E) and/or Asp (D).

56. An acylated analogue of human insulin, which analogue is [A8aar², A14Glu, B3aar¹, B26aar⁴, B27aar⁴, desB30] relative to human insulin; wherein aar¹ represents Glu (E), Gln (Q), Asp (D), Ser (S) or Thr (T); aar² represents His (H) or Arg (R); and aar⁴ independently of each other represent Glu (E) and/or Asp (D).

57. An acylated analogue of human insulin, which analogue is [A8aar², A14Glu, B3aar¹, B26aar⁴, B28aar⁴, desB30] relative to human insulin; wherein aar¹ represents Glu (E), Gln (Q), Asp (D), Ser (S) or Thr (T); aar² represents His (H) or Arg (R); and aar⁴ independently of each other represent Glu (E) and/or Asp (D).

58. An acylated analogue of human insulin, which analogue is [A8aar², A14Glu, B3aar¹, B27aar⁴, B28aar⁴, desB30] relative to human insulin; wherein aar¹ represents Glu (E), Gln (Q), Asp (D), Ser (S) or Thr (T); aar² represents His (H) or Arg (R); and aar⁴ independently of each other represent Glu (E) and/or Asp (D).

59. An acylated analogue of human insulin, which analogue is [A8aar², A21aar³, B3aar¹, B26aar⁴, desB30] relative to human insulin; wherein aar¹ represents Glu (E), Gln (Q), Asp (D), Ser (S) or Thr (T); aar² represents His (H) or Arg (R); aar³ represents Gly (G) or Ala (A); and aar⁴ represents Glu (E) and/or Asp (D).

60. An acylated analogue of human insulin, which analogue is [A8aar², A21aar³, B3aar¹, B27aar⁴, desB30] relative to human insulin; wherein aar¹ represents Glu (E), Gln (Q), Asp (D), Ser (S) or Thr (T); aar² represents His (H) or Arg (R); aar³ represents Gly (G) or Ala (A); and aar⁴ represents Glu (E) and/or Asp (D).

61. An acylated analogue of human insulin, which analogue is [A8aar², A21aar³, B3aar¹, B28aar⁴, desB30] relative to human insulin; wherein aar¹ represents Glu (E), Gln (Q), Asp (D), Ser (S) or Thr (T); aar² represents His (H) or Arg (R); aar³ represents Gly (G) or Ala (A); and aar⁴ represents Glu (E) and/or Asp (D).

62. An acylated analogue of human insulin, which analogue is [A8aar², A21aar³, B3aar¹, B28aar⁴, desB30] relative to human insulin; wherein aar¹ represents Glu (E); aar² represents His (H); aar³ represents Gly (G) or Ala (A); and aar⁴ represents Asp (D).

63. The acylated analogue of clause 61, wherein the [A8aar², A21aar³, B3aar¹, B28aar⁴, desB30] analogue of the invention is

A8H, A21A, B3E, B28D, B29K(N(eps)tetradecanedioyl-gGlu-2×OEG), desB30 human insulin; or

A8H, A21G, B3E, B28D, B29K(N(eps)tetradecanedioyl-gGlu-2×OEG), desB30 human insulin.

64. An acylated analogue of human insulin, which analogue is [A8aar², A21aar³, B3aar¹, B26aar⁴, B27aar⁴, desB30] relative to human insulin; wherein aar¹ represents Glu (E), Gln (Q), Asp (D), Ser (S) or Thr (T); aar² represents His (H) or Arg (R); aar³ represents Gly (G) or Ala (A); and aar⁴ independently of each other represent Glu (E) and/or Asp (D).

65. An acylated analogue of human insulin, which analogue is [A8aar², A21aar³, B3aar¹, B26aar⁴, B28aar⁴, desB30] relative to human insulin; wherein aar¹ represents Glu (E), Gln (Q), Asp (D), Ser (S) or Thr (T); aar² represents His (H) or Arg (R); aar³ represents Gly (G) or Ala (A); and aar⁴ independently of each other represent Glu (E) and/or Asp (D).

66. An acylated analogue of human insulin, which analogue is [A8aar², A21aar³, B3aar¹, B27aar⁴, B28aar⁴, desB30] relative to human insulin; wherein aar¹ represents Glu (E), Gln (Q), Asp (D), Ser (S) or Thr (T); aar² represents His (H) or Arg (R); aar³ represents Gly (G) or Ala (A); and aar⁴ independently of each other represent Glu (E) and/or Asp (D).

67. An acylated analogue of human insulin, which analogue is [A8aar², A21aar³, B3aar¹, B27aar⁴, B28aar⁴, desB30] relative to human insulin; wherein aar¹ represents Glu (E); aar² represents His (H); aar³ represents Gly (G) or Ala (A); and aar⁴ represents Glu (E).

68. The acylated analogue of clause 66, wherein the [A8aar², A21aar³, B3aar¹, B27aar⁴, B28aar⁴, desB30] analogue of the invention is

A8H, A21A, B3E, B27E, B28E, B29K(N(eps)tetradecanedioyl-gGlu-2×OEG), desB30 human insulin; or

A8H, A21G, B3E, B27E, B28E, B29K(N(eps)tetradecanedioyl-gGlu-2×OEG), desB30 human insulin.

69. An acylated analogue of human insulin, which analogue is [A14Glu, A21aar³, B3aar¹, B26aar⁴, desB30] relative to human insulin; wherein aar¹ represents Glu (E), Gln (Q), Asp (D), Ser (S) or Thr (T); aar³ represents Gly (G) or Ala (A); and aar⁴ represents Glu (E) and/or Asp (D).

70. An acylated analogue of human insulin, which analogue is [A14Glu, A21aar³, B3aar¹, B27aar⁴, desB30] relative to human insulin; wherein aar¹ represents Glu (E), Gln (Q), Asp (D), Ser (S) or Thr (T); aar³ represents Gly (G) or Ala (A); and aar⁴ represents Glu (E) and/or Asp (D).

71. An acylated analogue of human insulin, which analogue is [A14Glu, A21aar³, B3aar¹, B28aar⁴, desB30] relative to human insulin; wherein aar¹ represents Glu (E), Gln (Q), Asp (D), Ser (S) or Thr (T); aar³ represents Gly (G) or Ala (A); and aar⁴ represents Glu (E) and/or Asp (D).

72. An acylated analogue of human insulin, which analogue is [A14Glu, A21aar³, B3aar¹, B28aar⁴, desB30] relative to human insulin; wherein aar¹ represents Gln (Q); aar³ represents Ala (A); and aar⁴ represents Asp (D).

73. The acylated analogue of clause 71, wherein the [A14Glu, A21aar³, B3aar¹, B28aar⁴, desB30] analogue of the invention is

A14E, A21A, B3Q, B28D, B29K(N(eps)tetradecanedioyl-4×gGlu), desB30 human insulin.

74. An acylated analogue of human insulin, which analogue is [A14Glu, A21aar³, B3aar¹, B26aar⁴, B27aar⁴, desB30] relative to human insulin; wherein aar¹ represents Glu (E), Gln (Q), Asp (D), Ser (S) or Thr (T); aar³ represents Gly (G) or Ala (A); and aar⁴ independently of each other represent Glu (E) and/or Asp (D).

75. An acylated analogue of human insulin, which analogue is [A14Glu, A21aar³, B3aar¹, B26aar⁴, B28aar⁴, desB30] relative to human insulin; wherein aar¹ represents Glu (E), Gln (Q), Asp (D), Ser (S) or Thr (T); aar³ represents Gly (G) or Ala (A); and aar⁴ independently of each other represent Glu (E) and/or Asp (D).

76. An acylated analogue of human insulin, which analogue is [A14Glu, A21aar³, B³aar¹, B27aar⁴, B28aar⁴, desB30] relative to human insulin; wherein aar¹ represents Glu (E), Gln (Q), Asp (D), Ser (S) or Thr (T); aar³ represents Gly (G) or Ala (A); and aar⁴ independently of each other represent Glu (E) and/or Asp (D).

77. An acylated analogue of human insulin, which analogue is [A8aar², A14Glu, A21aar³, B3aar¹, B26aar⁴, desB30] relative to human insulin; wherein aar¹ represents Glu (E), Gln (Q), Asp (D), Ser (S) or Thr (T); aar² represents His (H) or Arg (R); aar³ represents Gly (G) or Ala (A); and aar⁴ represents Glu (E) and/or Asp (D).

78. An acylated analogue of human insulin, which analogue is [A8aar², A14Glu, A21aar³, B3aar¹, B27aar⁴, desB30] relative to human insulin; wherein aar¹ represents Glu (E), Gln (Q), Asp (D), Ser (S) or Thr (T); aar² represents His (H) or Arg (R); aar³ represents Gly (G) or Ala (A); and aar⁴ represents Glu (E) and/or Asp (D).

79. An acylated analogue of human insulin, which analogue is [A8aar², A14Glu, A21aar³, B3aar¹, B28aar⁴, desB30] relative to human insulin; wherein aar¹ represents Glu (E), Gln (Q), Asp (D), Ser (S) or Thr (T); aar² represents His (H) or Arg (R); aar³ represents Gly (G) or Ala (A); and aar⁴ represents Glu (E) and/or Asp (D).

80. An acylated analogue of human insulin, which analogue is [A8aar², A14Glu, A21aar³, B3aar¹, B26aar⁴, B27aar⁴, desB30] relative to human insulin; wherein aar¹ represents Glu (E), Gln (Q), Asp (D), Ser (S) or Thr (T); aar² represents His (H) or Arg (R); aar³ represents Gly (G) or Ala (A); and aar⁴ independently of each other represent Glu (E) and/or Asp (D).

81. An acylated analogue of human insulin, which analogue is [A8aar², A14Glu, A21aar³, B3aar¹, B27aar⁴, B28aar⁴, desB30] relative to human insulin; wherein aar¹ represents Glu (E), Gln (Q), Asp (D), Ser (S) or Thr (T); aar² represents His (H) or Arg (R); aar³ represents Gly (G) or Ala (A); and aar⁴ independently of each other represent Glu (E) and/or Asp (D).

82. An acylated analogue of human insulin, which analogue is

[A8H, A21A, B3E, B27E, B28E, desB30];

[A8H, A21A, B3E, B28D, desB30];

[A8H, A21G, B3E, B27E, B28E, desB30];

[A8H, A21G, B3E, B28D, desB30];

[A8H, B3E, B27E, B28E, desB30];

[A8H, B3E, B28D, desB30];

[A14E, A21A, B3Q, B28D, desB30;

[A14E, B3Q, B28D, desB30];

[A21A, B3E, B26E, desB30];

[A21A, B3E, B26E, B28E, desB30];

[A21A, B3E, B27E, B28E, desB30];

[A21A, B3E, B28D, desB30];

[A21A, B3E, B28E, desB30];

[A21A, B3Q, B28D, desB30];

[A21G, B3E, B26E, desB30];

[A21G, B3E, B26E, B28E, desB30];

[A21G, B3E, B27E, desB30];

[A21G, B3E, B27E, B28D, desB30];

[A21G, B3E, B27E, B28E, desB30];

[A21G, B3E, B28D, desB30];

[A21G, B3E, B28E, desB30];

[B3E, B26E, desB30];

[B3E, B26E, B28E, desB30];

[B3E, B27E, B28E, desB30];

[B3E, B28E, desB30];

[B3E, B28D, desB30];

[B3Q, B26E, desB30];

[B3Q, B28E, desB30]; or

[B3Q, B28D, desB30];

relative to human insulin.

83. An acylated analogue of human insulin, which analogue is derivatized by acylation of the epsilon amino group of the naturally occurring lysine residue at the B29 position with a group of Formula II

[Acyl]-[Linker]-

wherein the Linker group is an amino acid chain composed of from 1 to 10 amino acid residues selected from gGlu and/or OEG; wherein

gGlu represents a gamma glutamic acid residue;

OEG represents a residue of 8-amino-3,6-dioxaoctanoic acid (i.e. a group of the formula —NH—(CH₂)₂—O—(CH₂)₂—O—CH₂—CO—);

which amino acid residues may be present in any order; and

which amino acid chain comprises at least one gGlu residue; and

wherein the Acyl group is a residue of an α,ω-di-carboxylic acid selected from 1,14-tetradecanedioic acid; 1,15-pentadecanedioic acid; and 1,16-hexadecanedioic acid.

84. An acylated analogue of human insulin, wherein the Linker group according to Formula II above is an amino acid chain composed of from 1 to 8 amino acid residues selected from gGlu and/or OEG; which amino acid residues may be present in any order; and which amino acid chain comprises at least one gGlu residue.

85. An acylated analogue of human insulin, wherein the Linker group according to Formula II above is an amino acid chain composed of from 1 to 6 amino acid residues selected from gGlu and/or OEG.

86. An acylated analogue of human insulin, wherein the Linker group according to Formula II above is an amino acid chain composed of from 1 to 5 amino acid residues selected from gGlu and/or OEG.

87. An acylated analogue of human insulin, wherein the Linker group according to Formula II above is an amino acid chain composed of from 1 to 4 amino acid residues selected from gGlu and/or OEG.

88. An acylated analogue of human insulin, wherein the Linker group according to Formula II above is an amino acid chain composed of from 2 to 4 amino acid residues selected from gGlu and/or OEG.

89. An acylated analogue of human insulin, wherein the Linker group according to Formula II above is an amino acid chain composed of 3 or 4 amino acid residues selected from gGlu and/or OEG.

90. An acylated analogue of human insulin, wherein the Linker group according to Formula II above is an amino acid chain composed of four gGlu amino acid residues.

91. An acylated analogue of human insulin, wherein the Linker group according to Formula II above is an amino acid chain composed of one gGlu and two OEG amino acid residues.

92. An acylated analogue of human insulin, wherein the Acyl group according to Formula II above is a residue of an α,ω-di-carboxylic acid selected from 1,14-tetradecanedioic acid; 1,15-pentadecanedioic acid; and 1,16-hexadecanedioic acid.

93. An acylated analogue of human insulin, wherein the Acyl group according to Formula II above is a 1,14-tetradecanedioic acid residue.

94. An acylated analogue of human insulin, wherein the Acyl group according to Formula II above is a 1,15-pentadecanedioic acid residue.

95. An acylated analogue of human insulin, wherein the Acyl group according to Formula II above is a 1,16-hexadecanedioic acid residue.

96. An acylated analogue of human insulin, wherein the Linker group according to Formula II above is selected from tetradecanedioyl-gGlu-2×OEG; tetradecanedioyl-4×gGlu; hexadecanedioyl-gGlu-2×OEG; and hexadecanedioyl-4×gGlu.

97. An acylated analogue of human insulin, which analogue is

B3E, B26E, B29K(N(eps)tetradecanedioyl-4×gGlu), desB30 human insulin;

B3E, B26E, B29K(N(eps)tetradecanedioyl-gGlu-2×OEG), desB30 human insulin;

B3E, B26E, B28E, B29K(N(eps)tetradecanedioyl-4×gGlu), desB30 human insulin;

B3E, B26E, B28E, B29K(N(eps)tetradecanedioyl-gGlu-2×OEG), desB30 human insulin;

B3E, B27E, B28E, B29K(N(eps)tetradecanedioyl-4×gGlu), desB30 human insulin;

B3E, B27E, B28E, B29K(N(eps)tetradecanedioyl-gGlu-2×OEG), desB30 human insulin;

B3E, B28D, B29K(N(eps)tetradecanedioyl-4×gGlu), desB30 human insulin;

B3E, B28D, B29K(N(eps)tetradecanedioyl-gGlu-2×OEG), desB30 human insulin;

B3E, B28E, B29K(N(eps)tetradecanedioyl-4×gGlu), desB30 human insulin;

B3E, B28E, B29K(N(eps)tetradecanedioyl-gGlu-2×OEG), desB30 human insulin;

B3Q, B28D, B29K(N(eps)tetradecanedioyl-4×gGlu), desB30 human insulin;

B3Q, B28D, B29K(N(eps)tetradecanedioyl-gGlu-2×OEG), desB30 human insulin;

A8H, B3E, B27E, B28E, B29K(N(eps)tetradecanedioyl-gGlu-2×OEG), desB30 human insulin;

A8H, B3E, B28D, B29K(N(eps)tetradecanedioyl-4×gGlu), desB30 human insulin;

A8H, B3E, B28D, B29K(N(eps)tetradecanedioyl-gGlu-2×OEG), desB30 human insulin;

A8H, A21A, B3E, B27E, B28E, B29K(N(eps)tetradecanedioyl-gGlu-2×OEG), desB30 human insulin;

A8H, A21A, B3E, B28D, B29K(N(eps)tetradecanedioyl-gGlu-2×OEG), desB30 human insulin;

A8H, A21G, B3E, B27E, B28E, B29K(N(eps)tetradecanedioyl-gGlu-2×OEG), desB30 human insulin;

A8H, A21G, B3E, B28D, B29K(N(eps)tetradecanedioyl-gGlu-2×OEG), desB30 human insulin;

A14E, A21A, B3Q, B28D, B29K(N(eps)tetradecanedioyl-4×gGlu), desB30 human insulin;

A14E, B3Q, B28D, B29K(N(eps)tetradecanedioyl-4×gGlu), desB30 human insulin;

A21A, B3E, B26E, B29K(N(eps)tetradecanedioyl-gGlu-2×OEG), desB30 human insulin;

A21A, B3E, B26E, B29K(N(eps)tetradecanedioyl-4×gGlu), desB30 human insulin;

A21A, B3E, B26E, B28E, B29K(N(eps)tetradecanedioyl-4×gGlu), desB30 human insulin;

A21A, B3E, B26E, B28E, B29K(N(eps)tetradecanedioyl-gGlu-2×OEG), desB30 human insulin;

A21A, B3E, B27E, B28E, B29K(N(eps)tetradecanedioyl-4×gGlu), desB30 human insulin;

A21A, B3E, B27E, B28E, B29K(N(eps)hexadecanedioyl-4×gGlu), desB30 human insulin;

A21A, B3E, B27E, B28E, B29K(N(eps)tetradecanedioyl-gGlu-2×OEG), desB30 human insulin;

A21A, B3E, B28D, B29K(N(eps)tetradecanedioyl-4×gGlu), desB30 human insulin;

A21A, B3E, B28D, B29K(N(eps)tetradecanedioyl-gGlu-2×OEG), desB30 human insulin;

A21A, B3E, B28E, B29K(N(eps)tetradecanedioyl-4×gGlu), desB30 human insulin;

A21A, B3E, B28E, B29K(N(eps)tetradecanedioyl-gGlu-2×OEG), desB30 human insulin;

A21A, B3Q, B28D, B29K(N(eps)tetradecanedioyl-gGlu-2×OEG), desB30 human insulin;

A21G, B3E, B26E, B29K(N(eps)tetradecanedioyl-4×gGluG), desB30 human insulin;

A21G, B3E, B26E, B29K(N(eps)tetradecanedioyl-gGlu-2×OEG), desB30 human insulin;

A21G, B3E, B26E, B28E, B29K(N(eps)tetradecanedioyl-4×gGlu), desB30 human insulin;

A21G, B3E, B26E, B28E, B29K(N(eps)tetradecanedioyl-gGlu-2×OEG), desB30 human insulin;

A21G, B3E, B27E, B29K(N(eps)tetradecanedioyl-4×gGlu), desB30 human insulin;

A21G, B3E, B27E, B28D, B29K(N(eps)tetradecanedioyl-gGlu-2×OEG), desB30 human insulin;

A21G, B3E, B27E, B28E, B29K(N(eps)tetradecanedioyl-gGlu-2×OEG), desB30 human insulin;

A21G, B3E, B28D, B29K(N(eps)tetradecanedioyl-4×gGlu), desB30 human insulin;

A21G, B3E, B28D, B29K(N(eps)tetradecanedioyl-gGlu-2×OEG), desB30 human insulin;

A21G, B3E, B28D, B29K(N(eps)hexadecanedioyl-4×gGlu), desB30 human insulin;

A21G, B3E, B28D, B29K(N(eps)hexadecanedioyl-gGlu-2×OEG), desB30 human insulin;

A21G, B3E, B28E, B29K(N(eps)tetradecanedioyl-4×gGlu), desB30 human insulin;

A21G, B3E, B28E, B29K(N(eps)tetradecanedioyl-gGlu-2×OEG), desB30 human insulin;

B3Q, B26E, B29K(N(eps)tetradecanedioyl-4×gGlu), desB30 human insulin;

A21A, B3Q, B28E, B29K(N(eps)tetradecanedioyl-4×gGlu), desB30 human insulin;

B3Q, B28E, B29K(N(eps)tetradecanedioyl-4×gGlu), desB30 human insulin;

A21A, B3Q, B26E, B29K(N(eps)tetradecanedioyl-4×gGlu), desB30 human insulin;

A21A, B3E, B28E, B29K(N(eps)hexadecanedioyl-4×gGlu), desB30 human insulin; or

A21A, B3E, B28E, B29K(N(eps)hexadecanedioyl-gGlu-2×OEG), desB30 human insulin.

Any combination of two or more of the embodiments described herein is considered within the scope of the present invention.

### Definitions

### Nomenclature

Herein, the naming of the insulins is done according to the following principles:

The term “analogue” is frequently used for the insulin protein or peptide in question before it undergoes further chemical modification (derivatisation), and in particular acylation. The product resulting from such a chemical modification (derivatisation) is usually called a “derivative” or “acylated analogue”. However, in the context of this application, the term “analogue” designates analogues of human insulin as well as (the acylated) derivatives of such human insulin analogues.

The names are given as analogues, derivatives and modifications (acylations) relative to human insulin. For the naming of the acyl moiety (i.e. the [Acyl]-[Linker]- group of formula II), in some instances the naming is done according to IUPAC nomenclature, and in other instances the naming is done as peptide nomenclature.

As an example, the acyl moiety of the following structure (Chem. 1):

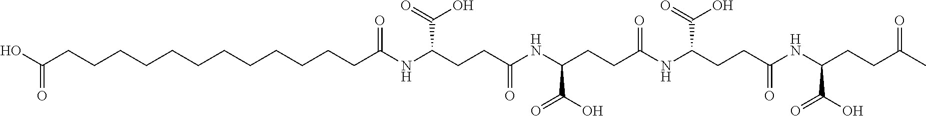 [SMILES: CC(=O)CC[C@H](NC(=O)CC[C@H](NC(=O)CC[C@H](NC(=O)CC[C@H](NC(=O)CCCCCCCCCCCCC(=O)O)C(=O)O)C(=O)O)C(=O)O)C(=O)O]

may be named “tetradecanedioyl-4×gGlu”, “tetradecanedioyl-4×γGlu” or, “1,14-tetradecanedioyl-4×gGlu” or the like, wherein γGlu (and gGlu) is short hand notation for the amino acid gamma glutamic acid in the L-configuration, and “4×” means that the residue following is repeated 4 times.

Similarly, the acyl moiety of the following structure (Chem. 2):

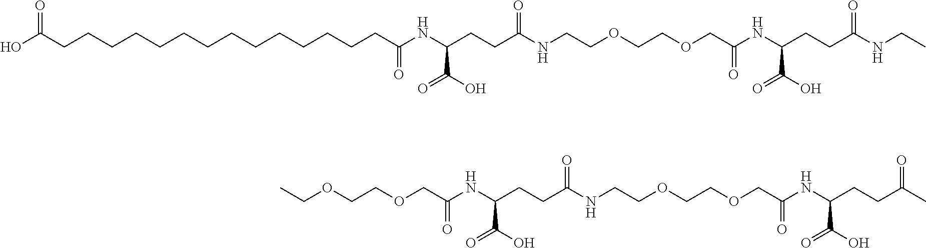

can for example be named “hexadecanedioyl-(gGlu-OEG)₃-gGlu)”, “hexadecanedioyl-(gGlu-OEG)₃-gGlu)”, “hexadecanedioyl-3×(gGlu-OEG)-gGlu)”, “1,16-hexadecanedioyl-(gGlu-OEG)₃-gGlu)”, “1,16-hexadecanedioyl-(gGlu-OEG)₃-gGlu)”, “1,16-hexadecanedioyl-3×(gGlu-OEG)-gGlu)”, “hexadecanedioyl-(Glu-OEG)₃-γGlu)”, “hexadecanedioyl-(γGlu-OEG)₃-γGlu)”, or “hexadecanedioyl-3×(γGlu-OEG)-γGlu)”;

wherein the mole of the following structure (Chem. 3)

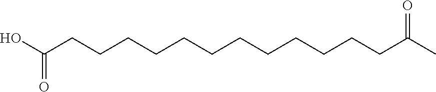 [SMILES: CC(=O)CCCCCCCCCCCCC(=O)O]

can for example be named tetradecanedioyl, 1,14-tetradecanedioyl or (short hand notation) C14 diacid. Similar notations apply for similar residues with 15 and 16 carbon atoms, pentadecanedioyl, C15 diacid, and hexadecanedioyl, C16 diacid, respectively.

γGlu (and gGlu) is short hand notation for the amino acid gamma glutamic acid H₂N—CH(CO₂H)—CH₂CH₂—CO₂H (connected via the alpha amino group and via the gamma (side chain) carboxy group), in the L-configuration.

OEG is short hand notation for the amino acid residue 8-amino-3,6-dioxaoctanoic acid, NH₂(CH₂)₂O(CH₂)₂OCH₂CO₂H.

“2×” and “3×” means that the residues following is repeated 2, respectively, 3 times.

For example, the insulin derivative of Example 1 is named “A21G, B3E, B28D, B29K(N(eps)tetradecanedioyl-gGlu-2×OEG), desB30 human insulin” to indicate that the lysine (K) in position B29 is modified by acylation on the epsilon nitrogen in the lysine residue of B29, denoted N^{ε} (or N(eps)) by the moiety tetradecanedioyl-Glu-2×OEG, the amino acid in position A21, N (aspargine) in human insulin, has been substituted with glycine (G), the amino acid in position B3, N in human insulin, has been substituted with glutamic acid, E, the amino acid in position B28, P (proline) in human insulin, has been substituted with aspartic acid (D), the amino acid in position B30, threonine, T, in human insulin, has been deleted. Asterisks in the formulae below indicate that the residue in question is different (i.e. substituted) as compared to human insulin.

Throughout this application, both formulas and names of preferred insulins of the invention are given.

In addition, the insulins of the invention are also named according to IUPAC nomenclature (OpenEye, IUPAC style). According to this nomenclature, the insulin derivative of Example 1 is assigned the following name: N{Epsilon-B29}-[2-[2-[2-[[2-[2-[2-[[(4S)-4-carboxy-4-(13-carboxytridecanoylamino)butanoyl]amino]ethoxy]-ethoxy]acetyl]amino]ethoxy]ethoxy]acetyl]-[GlyA21,GluB3,AspB28],des-ThrB30-Insulin(human).

It should be noted that formulas can be written with the lysine residue (that is modified by acylation) either is drawn with the lysine residue expanded (as shown e.g. in Example 5) or drawn with the lysine residue contracted (as shown e.g. in Example 1). In all cases the acyl group is attached to the epsilon nitrogen of the lysine residue.

### Physical Stability

The term “physical stability” of the insulin preparation as used herein refers to the tendency of the protein to form biologically inactive and/or insoluble aggregates of the protein as a result of exposure of the protein to thermo-mechanical stresses and/or interaction with interfaces and surfaces that are destabilizing, such as hydrophobic surfaces and interfaces. Physical stability of the aqueous protein preparations is evaluated by means of visual inspection and/or turbidity measurements after exposing the preparation filled in suitable containers (e.g. cartridges or vials) to mechanical/physical stress (e.g. agitation) at different temperatures for various time periods. Visual inspection of the preparations is performed in a sharp focused light with a dark background. A preparation is classified physically unstable with respect to protein aggregation, when it shows visual turbidity in daylight. Alternatively, the turbidity of the preparation can be evaluated by simple turbidity measurements well-known to the skilled person. Physical stability of the aqueous protein preparations can also be evaluated by using a spectroscopic agent or probe of the conformational status of the protein. The probe is preferably a small molecule that preferentially binds to a non-native conformer of the protein. One example of a small molecular spectroscopic probe of protein structure is Thioflavin T. Thioflavin T is a fluorescent dye that has been widely used for the detection of amyloid fibrils. In the presence of fibrils, and perhaps other protein configurations as well, Thioflavin T gives rise to a new excitation maximum at about 450 nm and enhanced emission at about 482 nm when bound to a fibril protein form. Unbound Thioflavin T is essentially non-fluorescent at the wavelengths.

### Chemical Stability

The term “chemical stability” of the protein preparation as used herein refers to changes in the covalent protein structure leading to formation of chemical degradation products with potential less biological potency and/or potential increased immunogenic properties compared to the native protein structure. Various chemical degradation products can be formed depending on the type and nature of the native protein and the environment to which the protein is exposed. Increasing amounts of chemical degradation products are often seen during storage and use of the protein preparation. Most proteins are prone to deamidation, a process in which the side chain amide group in glutaminyl or asparaginyl residues is hydrolysed to form a free carboxylic acid or asparaginyl residues to form an isoAsp derivative. Other degradations pathways involves formation of high molecular weight products where two or more protein molecules are covalently bound to each other through transamidation and/or disulfide interactions leading to formation of covalently bound dimer, oligomer and polymer degradation products (Stability of Protein Pharmaceuticals, Ahern T J & Manning M G, Plenum Press, New York 1992). Oxidation (of for instance methionine residues) can be mentioned as another variant of chemical degradation. The chemical stability of the protein preparation can be evaluated by measuring the amount of the chemical degradation products at various time-points after exposure to different environmental conditions (the formation of degradation products can often be accelerated by for instance increasing temperature). The amount of each individual degradation product is often determined by separation of the degradation products depending on molecule size, hydrofobicity, and/or charge using various chromatography techniques (e.g. SEC-HPLC and/or RP-HPLC). Since HMWP products are potentially immunogenic and not biologically active, low levels of HMWP are advantageous.

### Methods of Synthesis

The insulin derivatives of the invention may be obtained by conventional methods for the preparation of insulin, insulin analogues and insulin derivatives, and in particular the methods described in the working examples.

### Biological Activity

In another aspect the invention provides novel insulin derivatives for use as medicaments, or for use in the manufacture of medicaments or pharmaceutical compositions. The insulin analogue of the invention may in particular be useful as medicaments for the treatment of metabolic disorders.

The insulin derivatives of the invention are found to be short and fast acting insulin derivatives that are considered well suited for prandial use.

The insulin derivatives of the invention all possess insulin receptor affinities adequate for activating the insulin receptor in order to give the glycaemic response needed, i.e. being able to lower blood glucose in animals and humans. As a measure of functional (agonistic) activity of the insulins of the invention, lipogenesis activity in rat adipocytes are demonstrated.

The insulin derivatives of the invention are found to have a balanced insulin receptor (IR) to insulin-like growth factor 1 receptor (IGF-1R) affinity ratio (IR/IGF-1R).

In one aspect, the acylated insulin of the invention has an IR/IGF-1R ratio of above 0.5; of above 0.6; of above 0.7; of above 0.8; of above 0.9; of above 1; of above 1.5; or of above 2.

In another aspect, the acylated insulin analogue is a compound of the invention, wherein the Acyl group of Formula II is derived from 1,14-tetradecanedioic acid, and which acylated insulin analogue has a mean residence time (MRT) of less than 250 minutes; of less than 200 minutes; of less than 175 minutes; of less than 150 minutes; of less than 125 minutes; of less than 100 minutes; following subcutaneous injection of a 600 μM (approx.) formulation of the acylated insulin analogue of the invention, containing 1.6% (w/vol, approx.) glycerol and 30 mM phenol/m-cresol, pH 7.4, to pigs.

In another aspect, the acylated insulin analogue is a compound of the invention, wherein the Acyl group of Formula II is derived from 1,16-hexadecanedioic acid, and which acylated insulin analogue has a mean residence time (MRT) of less than 700 minutes; of less than 600 minutes; of less than 500 minutes; of less than 400 minutes; of less than 300 minutes; of less than 250 minutes; following subcutaneous injection of a 600 μM (approx.) formulation of the acylated insulin analogue of the invention, containing 1.6% (w/vol, approx.) glycerol and 30 mM phenol/m-cresol, pH 7.4, to pigs.

In a further aspect, the invention relates to the medical use of the acylated insulin analogue of the invention, and in particular to the use of such insulin derivatives for the treatment, prevention or alleviation of diseases, disorders or conditions relating to diabetes, Type 1 diabetes, Type 2 diabetes, impaired glucose tolerance, hyperglycemia, dyslipidemia, obesity, metabolic syndrome (metabolic syndrome X, insulin resistance syndrome), hypertension, cognitive disorders, atherosclerosis, myocardial infarction, stroke, cardiovascular disorders, coronary heart disease, inflammatory bowel syndrome, dyspepsia, or gastric ulcers, which method comprises administration to a subject in need thereof a therapeutically effective amount of the insulin derivative of the invention.

In another embodiment, the invention relates to the use of such insulin derivatives for the treatment, prevention or alleviation of diseases, disorders or conditions relating to diabetes, Type 1 diabetes, Type 2 diabetes, or impaired glucose tolerance, which method comprises administration to a subject in need thereof a therapeutically effective amount of the insulin derivative of the invention.

In a third embodiment, the invention relates to the use of such insulin derivatives for the treatment, prevention or alleviation of diseases, disorders or conditions relating to diabetes, and in particular Type 1 diabetes or Type 2 diabetes.

### Pharmaceutical Compositions

The present invention relates to acylated insulin analogues useful as medicaments, or for the manufacture of a pharmaceutical composition/medicament.

Therefore, in another aspect, the invention provides novel pharmaceutical compositions comprising a therapeutically effective amount of an insulin derivative according to the present invention.

The pharmaceutical composition according to the invention optionally comprises one or more pharmaceutically acceptable carriers and/or diluents.

The pharmaceutical composition of the present invention may further comprise other excipients commonly used in pharmaceutical compositions e.g. preservatives, chelating agents, tonicity agents, absorption enhancers, stabilizers, antioxidants, polymers, surfactants, metal ions, oleaginous vehicles and proteins (e.g., human serum albumin, gelatine or proteins).

In one embodiment of the invention the pharmaceutical composition of the invention is an aqueous preparation, i.e. preparation comprising water. Such preparation is typically a solution or a suspension. In a further embodiment of the invention the pharmaceutical composition is an aqueous solution.

The term “aqueous preparation” is defined as a preparation comprising at least 50% w/w water. Likewise, the term “aqueous solution” is defined as a solution comprising at least 50% w/w water, and the term “aqueous suspension” is defined as a suspension comprising at least 50% w/w water. Aqueous suspensions may contain the active compounds in admixture with excipients suitable for the manufacture of aqueous suspensions.

In one embodiment of the invention the insulin preparation comprises an aqueous solution of an insulin derivative of the present invention, wherein said insulin compound is present in a concentration from about 0.1 mM to about 20.0 mM; more particularly of from about 0.2 mM to about 4.0 mM; of from about 0.3 mM to about 2.5 mM; of from about 0.5 mM to about 2.5 mM; of from about 0.6 mM to about 2.0 mM; or of from about 0.6 mM to about 1.2 mM.

In another embodiment of the invention the insulin preparation comprises an aqueous solution of an insulin derivative of the present invention, wherein said insulin compound is present in a concentration of about 0.1 mM, of about 0.3 mM, of about 0.4 mM, of about 0.6 mM, of about 0.9 mM, of about 1.2 mM, of about 1.5 mM, or of about 1.8 mM

The pharmaceutical composition of the present invention may further comprise a buffer system. The buffer may be selected from the group consisting of, but not limited to, sodium acetate, sodium carbonate, sodium dihydrogen phosphate, disodium hydrogen phosphate, sodium phosphate, and tris(hydroxymethyl)-aminomethan, bicine, tricine, malic acid, glycyl-glycine, ethylene diamine, succinic acid, maleic acid, fumaric acid, tartaric acid, aspartic acid or mixtures thereof. Each one of these specific buffers constitutes an alternative embodiment of the invention.

In one embodiment the buffer is a phosphate buffer. In yet another embodiment, the concentration of said phosphate buffer is in the range from about 0.1 mM to 20 mM, in yet another embodiment the concentration of said phosphate buffer is in the range from 0.1 mM to about 10 mM, or from about 0.1 mM to about 8 mM, from about 1 mM to about 8 mM, or from about 2 mM to about 8 mM, or from 6 mM to 8 mM.

The pH of the injectable pharmaceutical composition of the invention is in the range of from 3 to 8.5. Preferably, the injectable pharmaceutical composition according to the invention has a pH in the range from about 6.8 to about 7.8.

In one embodiment the pH is in the range from about 7.0 to about 7.8, or from 7.2 to 7.6.

The insulin preparations of the present invention may further comprise a tonicity agent. The tonicity agent may be selected from the group consisting of a salt (e.g. sodium chloride), a sugar or sugar alcohol, an amino acid (e.g. L-glycine, L-histidine, arginine, lysine, isoleucine, aspartic acid, tryptophan, threonine), an alditol (e.g. glycerol (glycerine), 1,2-propanediol (propyleneglycol), 1,3-propanediol, 1,3-butanediol) polyethyleneglycol (e.g. PEG400), or mixtures thereof. Any sugar such as mono-, di-, or polysaccharides, or water-soluble glucans, including for example fructose, glucose, mannose, sorbose, xylose, maltose, lactose, sucrose, trehalose, dextran, pullulan, dextrin, cyclodextrin, soluble starch, hydroxyethyl starch and carboxymethylcellulose-Na may be used. In one embodiment the sugar additive is sucrose. Sugar alcohol includes, for example, mannitol, sorbitol, inositol, galactitol, dulcitol, xylitol, and arabitol. In one embodiment the sugar alcohol additive is mannitol. The sugars or sugar alcohols mentioned above may be used individually or in combination. Each one of these specific tonicity agents or mixtures hereof constitutes an alternative embodiment of the invention.

In a one embodiment of the invention, glycerol and/or mannitol and/or sodium chloride may be present in an amount corresponding to a concentration of from 0 to 250 mM, from 0 to 200 mM, or from 0 to 100 mM.

The insulin preparations of the present invention may further comprise a pharmaceutically acceptable preservative. The preservative may be present in an amount sufficient to obtain a preserving effect. The amount of preservative in a pharmaceutical composition of the invention may be determined from e.g. literature in the field and/or the known amount(s) of preservative in e.g. commercial products. Each one of these specific preservatives or mixtures hereof constitutes an alternative embodiment of the invention. The use of a preservative in pharmaceutical preparations is described, for example in Remington: The Science and Practice of Pharmacy, 19^{th} edition, 1995.

In one embodiment, the injectable pharmaceutical composition comprises at least one phenolic compound as preservative agent.

In another embodiment the phenolic compound for use according to the invention may be present in up to about 6 mg/mL of final injectable pharmaceutical composition, in particular of up to about 4 mg/mL of final injectable pharmaceutical composition.

In another embodiment the phenolic compound for use according to the invention may be present in an amount of up to about 4.0 mg/mL of final injectable pharmaceutical composition; in particular of from about 0.5 mg/mL to about 4.0 mg/mL; or of from about 0.6 mg/mL to about 4.0 mg/mL.

In another embodiment the preservative is phenol.

In another embodiment, the injectable pharmaceutical composition comprises a mixture of phenol and m-cresol as preservative agent.

In another embodiment, the injectable pharmaceutical composition comprises about 16 mM phenol (1.5 mg/mL) and about 16 mM m-cresol (1.72 mg/mL).

The pharmaceutical composition of the present invention may further comprise a chelating agent. The use of a chelating agent in pharmaceutical preparations is well-known to the skilled person. For convenience reference is made to Remington: The Science and Practice of Pharmacy, 19^{th} edition, 1995.

The pharmaceutical composition of the present invention may further comprise a absorption enhancer. The group of absorption enhancers may include but is not limited to nicotinic compounds. The term nicotinic compound includes nicotinamide, nicotinic acid, niacin, niacin amide and vitamin B3 and/or salts thereof and/or any combination thereof.

In one embodiment, the nicotinic compound is nicotinamide, and/or nicotinic acid, and/or a salt thereof. In another embodiment the nicotinic compound is nicotinamide. The nicotinic compound for use according to the invention may in particular be N-methyl nicotinamide, N,N-diethylnicotinamide, N-ethylnicotinamide, N,N-dimethylnicotinamide, N-propyl nicotinamide or N-butyl nicotinamide.

In another embodiment, the nicotinic compound is present in the amount of from about 5 mM to about 200 mM; in particular in the amount of from about 20 mM to about 200 mM; in the amount of from about 100 mM to about 170 mM; or in the amount of from about 130 mM to about 170 mM, such as about 130 mM, about 140 mM, about 150 mM, about 160 mM or about 170 mM.

The pharmaceutical composition of the present invention may further comprise a stabilizer. The term “stabilizer” as used herein refers to chemicals added to polypeptide containing pharmaceutical preparations in order to stabilize the peptide, i.e. to increase the shelf life and/or in-use time of such preparations. For convenience reference is made to Remington: The Science and Practice of Pharmacy, 19^{th} edition, 1995.

The pharmaceutical composition of the invention may further comprise an amount of an amino acid base sufficient to decrease aggregate formation by the polypeptide or protein during storage of the composition. The term “amino acid base” refers to an amino acid or a combination of amino acids, where any given amino acid is present either in its free base form or in its salt form. The amino acids may in particular be arginine, lysine, aspartic acid, glutamic acid, aminoguanidine, ornithine or N-monoethyl L-arginine, ethionine or buthionine, or S-methyl-L cysteine. In one embodiment of the invention the amino acid base may be present in an amount corresponding to a concentration of from 1 to 100 mM; of from 1 to 50 mM; or of from 1 to 30 mM.

In one embodiment, the pharmaceutical composition of the present invention may further comprise a surfactant. The term “surfactant” as used herein refers to any molecules or ions that are comprised of a water-soluble (hydrophilic) part, the head, and a fat-soluble (lipophilic) segment. Surfactants accumulate preferably at interfaces, which the hydrophilic part is orientated towards the water (hydrophilic phase) and the lipophilic part towards the oil- or hydrophobic phase (i.e. glass, air, oil etc.). The concentration at which surfactants begin to form micelles is known as the critical micelle concentration or CMC. Furthermore, surfactants lower the surface tension of a liquid. Surfactants are also known as amphipathic compounds. The use of a surfactant in pharmaceutical preparations is well-known to the skilled person. For convenience reference is made to Remington: The Science and Practice of Pharmacy, 19^{th} edition, 1995.

The invention further relates to a method for the preparation of such insulin preparations. The insulin preparations of this invention can be prepared by using any of a number of recognized methods. For example, the preparations can be prepared by mixing an aqueous solution of excipients with an aqueous solution of the insulin derivative, after which the pH is adjusted to a desired level and the mixture is made up to the final volume with water followed by sterile filtration.

### Zinc-Free Pharmaceutical Compositions

Insulin preparations traditionally comprise zinc added as e.g. the chloride or acetate salt to obtain an acceptable stability of the pharmaceutical preparation. However, it has surprisingly been found that the insulin derivatives of the invention, while maintaining a sufficient chemical and physical stability, may be formulated into pharmaceutical compositions without the addition of zinc, thereby giving a faster onset of action than comparable insulin analogues that need Zn²⁺ ions for maintaining sufficient chemical and physical stability. The zinc-free formulations are faster absorbed from the subcutaneous tissue, and thus allowing for prandial use.

In this respect it needs mentioning, that a zinc-free insulin pharmaceutical composition is indeed difficult to obtain, as traces of zinc, to a more or less extent, may be present in the excipients conventionally used for the manufacture of pharmaceutical compositions, and in particular in the rubber materials used in medical containers.

Therefore, in one aspect, the invention provides pharmaceutical compositions comprising an insulin derivative of the invention, formulated as a low-zinc composition, with no added zinc ions. Such pharmaceutical compositions are usually referred to as “zinc-free compositions”, although they may in fact be considered “low-zinc compositions”.

However, provided zinc-free excipients can be provided, the insulin derivatives of the present invention in fact allows for the preparation of zinc-free pharmaceutical compositions. Therefore, in another aspect, the invention provides zinc-free pharmaceutical compositions comprising an insulin derivative of the invention, and one or more pharmaceutically acceptable carriers or diluents, devoid of any zinc.

We have discovered that the B29K acylated insulin derivatives of the invention, holding a substitution in position B3, that adds to both the chemical and physical stability of pharmaceutical compositions formulated without addition of zinc-ions and with no added surfactants. Therefore, in a further aspect, the invention provides a low-zinc or zinc-free pharmaceutical composition as described above, comprising an insulin derivative of the invention comprising an additional substitution in position B3 (i.e. B3E or B3Q), and one or more pharmaceutically acceptable carriers or diluents, in which pharmaceutical composition, however, no surfactant has been added.

In one embodiment, the invention provides a low-zinc pharmaceutical composition as described above, wherein the zinc ions may be present in an amount corresponding to a concentration of less than 0.2 Zn²⁺ ions per 6 insulin molecules; of less than 0.15 Zn²⁺ ions per 6 insulin molecules; of less than 0.12 Zn²⁺ ions per 6 insulin molecules; 0.1 Zn²⁺ ions per 6 insulin molecules; of less than 0.09 Zn²⁺ ions per 6 insulin molecules; of less than 0.08 Zn²⁺ ions per 6 insulin molecules; of less than 0.07 Zn²⁺ ions per 6 insulin molecules; of less than 0.06 Zn²⁺ ions per 6 insulin molecules; of less than 0.05 Zn²⁺ ions per 6 insulin molecules; of less than 0.04 Zn²⁺ ions per 6 insulin molecules; of less than 0.03 Zn²⁺ ions per 6 insulin molecules; of less than 0.02 Zn²⁺ ions per 6 insulin molecules; or of less than 0.01 Zn²⁺ ions per 6 insulin molecules.

In another embodiment, the invention provides a pharmaceutical composition formulated as a low-zinc composition, with no added zinc ions, comprising an insulin derivative and one or more pharmaceutically acceptable carriers or diluents.

In a further embodiment, the invention provides a pharmaceutical composition formulated as a low-zinc composition as described above, and wherein no surfactant has been added.

In an even further embodiment, the invention provides a pharmaceutical composition formulated as a low-zinc composition as described above, and wherein no surfactant has been added, and comprising a nicotinic compound, and in particular nicotinamide, as described above.

### Methods of Administration

The pharmaceutical composition of the invention may be administered by conventional methods.

Parenteral administration may be performed by subcutaneous, intramuscular, intraperitoneal or intravenous injection by means of a syringe, optionally a pen-like syringe. Alternatively, parenteral administration can be performed by means of an infusion pump. As a further option, the insulin preparations containing the insulin compound of the invention can also be adapted to transdermal administration, e.g. by needle-free injection or from a microneedle patch, optionally an iontophoretic patch, or transmucosal, e.g. buccal, administration.

The pharmaceutical composition of the invention may be administered to a patient in need of such treatment at several sites, for example, at topical sites, for example, skin and mucosal sites, at sites which bypass absorption, for example, administration in an artery, in a vein, in the heart, and at sites which involve absorption, for example, administration in the skin, under the skin, in a muscle or in the abdomen.

The pharmaceutical composition of the invention may be used in the treatment of diabetes by parenteral administration. The actual dosage depends on the nature and severity of the disease being treated, and is within the discretion of the physician, and may be varied by titration of the dosage to the particular circumstances of this invention to produce the desired therapeutic effect. However, it is currently contemplated that the insulin derivative according to the invention shall be present in the final pharmaceutical composition in an amount of from about 0.1 mM to about 20.0 mM; more particularly of from about 0.2 mM to about 4.0 mM; of from about 0.3 mM to about 2.5 mM; of from about 0.5 mM to about 2.5 mM; of from about 0.6 mM to about 2.0 mM; or of from about 0.6 mM to about 1.2 mM.

The pharmaceutical compositions of the invention may also be prepared for use in various medical devices normally used for the administration of insulin, including pen-like devices used for insulin therapy by injection, continuous subcutaneous insulin infusion therapy by use of pumps, and/or for application in basal insulin therapy.

In one embodiment the pharmaceutical composition of the invention is formulated into a pen-like device for use for insulin therapy by injection.

In another embodiment the pharmaceutical composition of the invention is formulated into an external pump for insulin administration.

### Methods of Therapy

The present invention relates to drugs for therapeutic use. More specifically the invention relates to the use of the acylated derivatives of human insulin analogues of the invention for the treatment or prevention of medical conditions relating to diabetes.

Therefore, in another aspect, the invention provides a method for the treatment or alleviation of a disease or disorder or condition of a living animal body, including a human, which disease, disorder or condition may be selected from a disease, disorder or condition relating to diabetes, Type 1 diabetes, Type 2 diabetes, impaired glucose tolerance, hyperglycemia, dyslipidemia, obesity, metabolic syndrome (metabolic syndrome X, insulin resistance syndrome), hypertension, cognitive disorders, atherosclerosis, myocardial infarction, stroke, cardiovascular disorders, coronary heart disease, inflammatory bowel syndrome, dyspepsia, or gastric ulcers, which method comprises the step of administering to a subject in need thereof a therapeutically effective amount of the acylated analogue of human insulin of the invention.

In another embodiment the invention provides a method for the treatment or alleviation of a disease or disorder or condition of a living animal body, including a human, which disease, disorder or condition may be selected from a disease, disorder or condition relating to diabetes, Type 1 diabetes, Type 2 diabetes, impaired glucose tolerance, hyperglycemia, dyslipidemia, obesity, metabolic syndrome (metabolic syndrome X, insulin resistance syndrome), hypertension, cognitive disorders, atherosclerosis, myocardial infarction, stroke, cardiovascular disorders, coronary heart disease, inflammatory bowel syndrome, dyspepsia, or gastric ulcers, which method comprises administration to a subject in need thereof a therapeutically effective amount of the acylated analogue of human insulin of the invention.

In a third embodiment the invention provides a method for the treatment or alleviation of a disease or disorder or condition of a living animal body, including a human, which disease, disorder or condition may be selected from a disease, disorder or condition relating to diabetes, Type 1 diabetes, Type 2 diabetes, impaired glucose tolerance, hyperglycemia, dyslipidemia, obesity, or metabolic syndrome (metabolic syndrome X, insulin resistance syndrome).

In a fourth embodiment the invention provides a method for the treatment or alleviation of a disease or disorder or condition of a living animal body, including a human, which disease, disorder or condition may be selected from a disease, disorder or condition relating to diabetes, in particular Type 1 diabetes, or Type 2 diabetes.

### BRIEF DESCRIPTION OF THE DRAWINGS

The present invention is further illustrated by reference to the accompanying drawing, in which:

FIGS. 1A, 1B and 1C show a schematic illustration of the fibrillation process when measured in the “ThT fibrillation assay” described herein;

FIGS. 2A and 2B show PK profiles of analogues of the invention (Examples 17 and 20, and Examples 3, 13 and 21, respectively), and of analogues of the prior art (Prior Art Analogues 2, 3 and 4, and Prior Art Analogue 4, respectively), following subcutaneous injection to Sprague Dawley rats;

FIG. 2C1 (0-180 minutes) and FIG. 2C2 (0-30 minutes) show PD profiles of analogues of the invention (Examples 17 and 20), and of analogues of the prior art (Prior Art Analogues 2, 3, and 4);

FIG. 2D1 (0-180 minutes) and FIG. 2D2 (0-30 minutes) show PD profiles of analogues of the invention (Examples 3, 13 and 21), and of analogues of the prior art (Prior Art Analogues 4), following subcutaneous injection to Sprague Dawley rats;

FIGS. 3A1 (0-600 minutes), 3A2 (0-60 minutes), 3B1 (0-600 minutes) and 3B2 (0-60 minutes) show the PD (pharmacodynamic) and the PK (pharmacokinetic) profiles of the insulin derivative of Example 16, i.e. A21G, B3E, B28D, B29K(N(eps) tetradecanedioyl-4×gGlu), desB30 human insulin, formulated with 0 zinc per 6 insulin molecules, and the resulting changes in plasma glucose, and the insulin concentrations vs. time, respectively (1 nmol/kg) following subcutaneous injection to LYD pigs;

FIGS. 4A1 (0-600 minutes), 4A2 (0-60 minutes), 4B1 (0-600 minutes) and 4B2 (0-60 minutes) show the PD (pharmacodynamic) and the PK (pharmacokinetic) profiles of the insulin derivative of Example 21, i.e. A21A, B3E, B27E, B28E, B29K(N(eps) tetradecanedioyl-4×gGlu), desB30 human insulin, formulated with 0 zinc per 6 insulin molecules, and the resulting changes in plasma glucose, and the insulin concentrations vs. time, respectively (1 nmol/kg) following subcutaneous injection to LYD pigs; and

FIGS. 5A1 (0-720 minutes), 5A2 (0-120 minutes), 5B1 (0-720 minutes) and 5B2 (0-120 minutes) show the PD (pharmacodynamic) and the PK (pharmacokinetic) profiles of the insulin derivative of Prior Art Analogue 2, i.e. B28D, B29K(N(eps) tetradecanedioyl-4×gGlu), desB30 human insulin, formulated as described above with 0 or 3 zinc per 6 insulin molecules, and the resulting changes in plasma glucose, and the insulin concentrations vs. time, respectively (1 nmol/kg) following subcutaneous injection to LYD pigs.

### EXAMPLES

The invention is further illustrated with reference to the following examples, which are not intended to be in any way limiting to the scope of the invention as claimed.

### Insulin Analogue Expression and Purification

### Insulin Analogue Expression

The insulin analogue, i.e. the two-chain non-acylated insulin analogues, for use according to the invention are produced recombinantly by expressing a DNA sequence encoding the insulin analogue in question in a suitable host cell by well-known techniques, e.g. as disclosed in U.S. Pat. No. 6,500,645. The insulin analogue is either expressed directly or as a precursor molecule which may have an N-terminal extension on the B-chain and/or a connecting peptide (C-peptide) between the B-chain and the A-chain. This N-terminal extension and C-peptide are cleaved off in vitro by a suitable protease, e.g. Achromobactor lyticus protease (ALP) or trypsin, and will therefore have a cleavage site next to position B1 and A1, respectively. N-terminal extensions and C-peptides of the type suitable for use according to this invention are disclosed in e.g. U.S. Pat. No. 5,395,922, EP 765395 and WO 9828429.

The polynucleotide sequence encoding the insulin analogue precursor for use according to the invention may be prepared synthetically by established methods, e.g. the phosphoamidite method described by Beaucage et al. (1981) Tetrahedron Letters 22 1859-1869, or the method described by Matthes et al. (1984) EMBO Journal 3 801-805. According to the phosphoamidite method, oligonucleotides are synthesised in e.g. an automatic DNA synthesiser, purified, duplexed, and ligated to form the synthetic DNA construct. A currently preferred way of preparing the DNA construct is by polymerase chain reaction (PCR).

The recombinant method will typically make use of a vector which is capable of replicating in the selected microorganism or host cell and which carries a polynucleotide sequence encoding the insulin analogue precursor for use according to the present invention. The recombinant vector may be an autonomously replicating vector, i.e., a vector which exists as an extra-chromosomal entity, the replication of which is independent of chromosomal replication, e.g. a plasmid, an extra-chromosomal element, a mini-chromosome, or an artificial chromosome. The vector may contain any means for assuring self-replication. Alternatively, the vector may be one which, when introduced into the host cell, is integrated into the genome and replicated together with the chromosome(s) into which it has been integrated. Furthermore, a single vector or plasmid or two or more vectors or plasmids which together contain the total DNA to be introduced into the genome of the host cell, or a transposon may be used. The vector may be linear or closed circular plasmids and will preferably contain an element(s) that permits stable integration of the vector into the host cell's genome or autonomous replication of the vector in the cell independent of the genome.

The recombinant expression vector may be one capable of replicating in yeast. Examples of sequences which enable the vector to replicate in yeast are the yeast plasmid 2 μm replication genes REP 1-3 and origin of replication.

The vector may contain one or more selectable markers, which permit easy selection of trans-formed cells. A selectable marker is a gene the product, which provides for biocide or viral resistance, resistance to heavy metals, prototrophy to auxotrophs, and the like. Examples of bacterial selectable markers are the dal genes from Bacillus subtilis or Bacillus licheniformis, or markers which confer antibiotic resistance such as ampicillin, kanamycin, chloramphenicol or tetracycline resistance. Selectable markers for use in a filamentous fungal host cell include amdS (acetamidase), argB (orni-thine carbamoyltransferase), pyrG (orotidine-5′-phosphate decarboxylase) and trpC (anthranilate syn-thase. Suitable markers for yeast host cells are ADE2, HIS3, LEU2, LYS2, MET3, TRP1, and URA3. A well suited selectable marker for yeast is the Schizosaccharomyces pompe TPI gene (Russell (1985) Gene 40 125-130).

In the vector, the polynucleotide sequence is operably connected to a suitable promoter sequence. The promoter may be any nucleic acid sequence which shows transcriptional activity in the host cell of choice including mutant, truncated, and hybrid promoters, and may be obtained from genes encoding extra-cellular or intra-cellular polypeptides either homologous or heterologous to the host cell.

Examples of suitable promoters for directing the transcription in a bacterial host cell, are the promoters obtained from the E. coli lac operon, Streptomyces coelicolor agarase gene (dagA), Bacillus subtilis levansucrase gene (sacB), Bacillus licheniformis alpha-amylase gene (amyL), Bacillus stearothermophilus maltogenic amylase gene (amyM), Bacillus amyloliquefaciens alpha-amylase gene (amyQ), and Bacillus licheniformis penicillinase gene (penP). Examples of suitable promoters for di-recting the transcription in a filamentous fungal host cell are promoters obtained from the genes for Aspergillus oryzae TAKA amylase, Rhizomucor miehei aspartic proteinase, Aspergillus niger neutral alpha-amylase, and Aspergillus niger acid stable alpha-amylase. In a yeast host, useful promoters are the Saccharomyces cerevisiae Ma1, TPI, ADH, TDH3 or PGK promoters.

The polynucleotide sequence encoding the insulin peptide backbone for use according to the invention also will typically be operably connected to a suitable terminator. In yeast, a suitable terminator is the TPI terminator (Alber et al. (1982) J. Mol. Appl. Genet. 1 419-434).

The procedures used to combine the polynucleotide sequence encoding the insulin analogue for use according to the invention, the promoter and the terminator, respectively, and to insert them into a suitable vector containing the information necessary for replication in the selected host, are well known to persons skilled in the art. It will be understood that the vector may be constructed either by first preparing a DNA construct containing the entire DNA sequence encoding the insulin backbones for use according to the invention, and subsequently inserting this fragment into a suitable expression vector, or by sequentially inserting DNA fragments containing genetic information for the individual elements (such as the signal and pro-peptide (N-terminal extension of the B-chain), C-peptide, A- and B-chains), followed by ligation.

The vector comprising the polynucleotide sequence encoding the insulin analogue for use according to the invention is introduced into a host cell, so that the vector is maintained as a chromosomal integrant, or as a self-replicating extra-chromosomal vector. The term “host cell” encompasses any progeny of a parent cell that is not identical to the parent cell due to mutations that occur during replication. The host cell may be a unicellular microorganism, e.g. a prokaryote, or a non-unicellular microorganism, e.g. a eukaryote. Useful unicellular cells are bacterial cells such as gram positive bacteria including, but not limited to, a Bacillus cell, a Streptomyces cell, or a gram negative bacteria such as E. coli and Pseudomonas sp. Eukaryote cells may be mammalian, insect, plant, or fungal cells.

The host cell may in particular be a yeast cell. The yeast organism may be any suitable yeast organism which, on cultivation, secretes the insulin peptide backbone or the precursor hereof into the culture medium. Examples of suitable yeast organisms are include strains selected from Saccharomyces cerevisiae, Saccharomyces kluyveri, Schizosaccharomyces pombe, Sacchoromyces uvarum, Kluyveromyces lactis, Hansenula polymorpha, Pichia pastoris, Pichia methanolica, Pichia kluyveri, Yarrowia lipolytica, Candida sp., Candida utilis, Candida cacaoi, Geotrichum sp., and Geotrichum fermentans.

The transformation of the yeast cells may for instance be effected by protoplast formation followed by transformation by known methods. The medium used to cultivate the cells may be any conventional medium suitable for growing yeast organisms.

### Insulin Analogue Purification

The secreted insulin analogue or precursor hereof may be recovered from the medium by conventional procedures including separating the yeast cells from the medium by centrifugation, by filtration or by catching or adsorbing the insulin analogue or precursor hereof on an ion exchange matrix or on a reverse phase absorption matrix, precipitating the proteinaceous components of the supernatant, or by filtration by means of a salt, e.g. ammonium sulphate, followed by purification by a variety of chromatographic procedures, e.g. ion exchange chromatography, affinity chromatography, etc.

The purification and digestion of the insulin peptide backbones of this invention is carried out as follows:

The single-chain insulin analogue precursor, which may contain an N-terminal extension of the B-chain and a modified C-peptide between the B-chain and the A-chain, is purified and concentrated from the yeast culture supernatant by cation exchange (Kjeldsen et al. (1998) Prot. Expr. Pur. 14 309-316).

The single-chain insulin analogue precursor is matured into two-chain insulin peptide backbone by digestion with lysine-specific immobilised ALP (Kristensen et al. (1997) J. Biol. Chem. 20 12978-12983) or by use of trypsin to cleave off the N-terminal extension of the B-chain, if present, and the C-peptide.

### ALP Digestion

The eluate from the cation exchange chromatography step containing the insulin peptide backbone precursor is diluted with water to an ethanol concentration of 15-20%. Sodium glutamate is added to a concentration of 15 mM and pH is adjusted to 9.7 by NaOH. Immobilised ALP (4 gram/L) is added in a proportion of 1:100 (volume:volume) and digestion is allowed to proceed with mild stirring in room temperature overnight.

The digestion reaction is analysed by analytical LC on a Waters Acquity Ultra-Performance Liquid Chromatography system using a C18 column and the molecular weight is confirmed by matrix-assisted laser desorption ionisation time-of-flight (MALDI-TOF) mass spectrometry (MS) (Bruker Daltonics Autoflex II TOF/TOF).

The immobilised ALP is removed by filtration using a 0.2 μm filter. The two-chain insulin peptide backbone is purified by reversed phase HPLC (Waters 600 system) on a C18 column using an acetonitrile gradient. The desired insulin is recovered by lyophilisation.

Purity is determined by analytical LC on a Waters Acquity Ultra-Performance Liquid Chromatography system using a C18 column, and the molecular weight is confirmed by MALDI-TOF MS.

### Abbreviations

ALP—Achromobactor lyticus protease

C-peptide—connecting peptide

HPLC—high-performance liquid chromatography

IR—insulin receptor

IGF-1R insulin-like growth factor 1 receptor

LC—liquid chromatography

MALDI-TOF—matrix-assisted laser desorption ionisation time-of-flight

MS—mass spectrometry

PCR—polymerase chain reaction

PD—pharmacodynamics (blood/plasma glucose lowering effect)

PG—plasma glucose

PK—pharmacodynamics (blood/plasma insulin concentrations versus time profiles)

tBu—tert-butyl;

DCM—dichloromethane;

DIC—diisopropylcarbodiimide;

DIPEA=DIEA—N,N-disopropylethylamine;

DMF—N,N-dimethylformamide;

DMSO—dimethyl sulphoxide;

EtOAc—ethyl acetate;

Fmoc—9-fluorenylmethyloxycarbonyl;

γGlu (gGlu)—gamma L-glutamyl;

HCl—hydrochloric acid;

HOBt—1-hydroxybenzotriazole;

NMP—N-methylpyrrolidone;

MeCN—acetonitrile;

OEG—[2-(2-aminoethoxy)ethoxy]ethylcarbonyl;

Su—succinimidyl-1-yl=2,5-dioxo-pyrrolidin-1-yl;

OSu—succinimidyl-1-yloxy=2,5-dioxo-pyrrolidin-1-yloxy;

RPC—reverse phase chromatography;

RT—room temperature;

TCTU—O-(6-chloro-benzotriazol-1-yl)-N,N,N′,N′-tetramethyluronium tetrafluoroborate;

TFA—trifluoroacetic acid;

THF—tetrahydrofuran;

TNBS—2,4,6-trinitrobenzenesulfonic acid;

TRIS—tris(hydroxymethyl)aminomethane; and

TSTU—O—(N-succinimidyl)-1,1,3,3-tetramethyluronium tetrafluoroborate.

### Pharmacokinetic (PK) Parameters

T_{1/2}—terminal halflife;

MRT—mean residence time;

F—bioavailability (fraction absorbed);

Tₘₐₓ—time to maximal plasma exposure;

Cₘₐₓ—maximal plasma concentration;

D—dose;

Cₘₐₓ/D—dose-normalised maximal plasma concentration;

AUC—area under the curve;

AUC/D—dose-normalised area under the curve;

% extrap—the percentage of extrapolated profile.

### General Remarks

The following examples and general procedures refer to intermediate compounds and final products identified in the specification and in the synthesis schemes. The preparation of the compounds of the present invention is described in detail using the following examples, but the chemical reactions described are disclosed in terms of their general applicability to the preparation of compounds of the invention.

Occasionally, the reaction may not be applicable as described to each compound included within the disclosed scope of the invention. The compounds for which this occurs will be readily recognised by those skilled in the art. In these cases the reactions can be successfully performed by conventional modifications known to those skilled in the art, i.e. by appropriate protection of interfering groups, by changing to other conventional reagents, or by routine modification of reaction conditions.

Alternatively, other reactions disclosed herein or otherwise conventional will be applicable to the preparation of the corresponding compounds of the invention. In all preparative methods, all starting materials are known or may easily be prepared from known starting materials. All temperatures are set forth in degrees Celsius and unless otherwise indicated, all parts and percentages are by weight when referring to yields and all parts are by volume when referring to solvents and eluents.

The compounds of the invention can be purified by employing one or more of the following procedures which are typical within the art. These procedures can—if needed—be modified with regard to gradients, pH, salts, concentrations, flow, columns and so forth. Depending on factors such as impurity profile, solubility of the insulins in question etcetera, these modifications can readily be recognised and made by a person skilled in the art.

After acidic HPLC or desalting, the compounds are isolated by lyophilisation of the pure fractions.

After neutral HPLC or anion exchange chromatography, the compounds are desalted, precipitated at isoelectrical pH, or purified by acidic HPLC.

### Typical Purification Procedures

### RP-HPLC System:

Gilson system consisting of the following: Liquid handler Model 215, Pump Model 322-H2 and UV Detector Model 155 (UV 215 nm and 280 nm).

### Anion Exchange and Desalting System:

Äkta Explorer system consists of the following: Pump Model P-900, UV detector Model UV-900 (UV 214, 254 and 280 nm), pH and conductivity detector Model pH/C-900, Fraction collector Model Frac-950.

### Acidic RP-HPLC:

### Column: Phenomenex Gemini, 5 μM 5 u C18 110 Å, 30×250 mm

Flow: 20 mL/min
Buffer A: 0.1% TFA in water
Buffer B: 0.1% TFA in acetonitrile

### Neutral RP-HPLC:

### Column: Phenomenex Gemini, 5 μM 5 u C18 110 Å, 30×250 mm

Flow: 20 mL/min
Buffer A: 10 mM Tris, 15 mM (NH₄)₂SO₄, pH=7.3, 20% acetonitrile in milliQ
Buffer B: 20% milliQ in acetonitrile

### Anion Exchange Chromatography:

### Column-material: Poros50HQ or Source30Q

Flow: Column dependent
Buffer A: 15 mM Tris, 25 mM NH₄OAc, 50% EtOH, pH=7.5.
Buffer B: 15 mM Tris, 500 mM NH₄OAc, 50% EtOH, pH=7.5.

### Desalting:

### Column: HiPrep 26/10

Flow: 20 mL/min
Buffer A: 0.1% TFA in water
Buffer B: 0.1% TFA in acetonitrile

Acylation reagents were synthesized either in solution or on solid phase essentially as described in e.g. WO 2009/115469.

### General Procedure for the Solid Phase Synthesis of Acylation Reagents of the General Formula III

[Acyl]-[Linker]-Act

wherein the Acyl and Linker groups are as defined above, and Act is the leaving group of an active ester, such as N-hydroxysuccinimide (OSu), or 1-hydroxybenzotriazole, and

wherein carboxylic acids within the Acyl and Linker moieties of the acyl moiety are protected as tert-butyl esters.

Compounds of the general Formula III may be synthesised on solid support using procedures in the art of solid phase peptide synthesis known to the skilled person.

One such procedure comprises attachment of a Fmoc protected amino acid to a polystyrene 2-chlorotritylchloride resin. The attachment may be accomplished using the free N-protected amino acid in the presence of a tertiary amine, like triethyl amine or N,N-diisopropylethylamine (see references below). The C-terminal end (which is attached to the resin) of this amino acid is at the end of the synthetic sequence being coupled to the parent insulins of the invention.

After attachment of the Fmoc amino acid to the resin, the Fmoc group is deprotected using, e.g., secondary amines, like piperidine or diethyl amine, followed by coupling of another (or the same) Fmoc protected amino acid and deprotection. The synthetic sequence is terminated by coupling of mono-tert-butyl protected fatty (α, ω) diacids, like hexadecanedioic, pentadecanedioic, or tetradecanedioic acid mono-tert-butyl esters.

Cleavage of the compounds from the resin is accomplished using diluted acid like 0.5-5% TFA/DCM (trifluoroacetic acid in dichloromethane), acetic acid (e.g. 10% in DCM, or HOAc/triflouroethanol/DCM 1:1:8), or hecafluoroisopropanol in DCM (see e.g. F. Z. Dörwald: Organic Synthesis on Solid Phase; Wiley-VCH 2000, ISBN 3-527-29950-5; N. Sewald & H.-D. Jakubke: Peptides: Chemistry and Biology; Wiley-VCH, 2002, ISBN 3-527-30405-3; or The Combinatorial Chemistry Catalog, 1999, Novabiochem AG, and references cited therein). This ensures that tert-butyl esters present in the compounds as carboxylic acid protecting groups are not deprotected.

Finally, the C-terminal carboxy group (liberated from the resin) is activated, e.g., as the N-hydroxysuccinimide ester (OSu). This activated ester is deprotected, e.g. using neat TFA, and used either directly or after purification (crystallisation) as coupling reagent in attachment to parent insulins of the invention. This procedure is illustrated below.

### General Procedure for Synthesis of Acylation Reagent on Solid Phase:

### Synthesis of tetradecanedioyl-4×gGlu-OSu (Chem. 4)

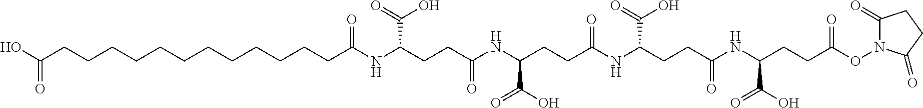 [SMILES: O=C(O)CCCCCCCCCCCCC(=O)N[C@@H](CCC(=O)N[C@@H](CCC(=O)N[C@@H](CCC(=O)N[C@@H](CCC(=O)ON1C(=O)CCC1=O)C(=O)O)C(=O)O)C(=O)O)C(=O)O]

2-Chlorotrityl resin 100-200 mesh 1.5 mmol/g (15.79 g, 23.69 mmol) was left to swell in dry dichloromethane (150 mL) for 20 minutes. A solution of Fmoc-Glu-OtBu (6.72 g, 15.79 mmol) and N,N-diisopropylethylamine (10.46 mL, 60.01 mmol) in dry dichloromethane (120 mL) was added to resin and the mixture was shaken for 16 hrs. Resin was filtered and treated with a solution of N,N-diisopropylethylamine (5.5 mL, 31.59 mmol) in methanol/dichloromethane mixture (9:1, 150 mL, 5 min). Then resin was washed with N,N-dimethylformamide (2×150 mL), dichloromethane (2×150 mL) and N,N-dimethylformamide (2×150 mL).

Fmoc group was removed by treatment with 20% piperidine in N,N-dimethylformamide (2×150 mL, 1×5 min, 1×20 min). Resin was washed with N,N-dimethylformamide (2×150 mL), 2-propanol (2×150 mL), dichloromethane (2×150 mL) and N,N-dimethylformamide (2×150 mL). Solution of Fmoc-Glu-OtBu (10.08 g, 23.69 mmol), O-(6-chloro-benzotriazol-1-yl)-N,N,N′,N′-tetramethyluronium tetrafluoroborate (TCTU, 8.42 g, 23.69 mmol) and N,N-diisopropylethylamine (7.43 mL, 42.64 mmol) in N,N-dimethylformamide (120 mL) was added to resin and mixture was shaken for 16 hr. Resin was filtered and treated with a solution of N,N-diisopropylethylamine (5.5 mL, 31.59 mmol) in methanol/dichloromethane mixture (9:1, 150 mL, 5 min). Then resin was washed with N,N-dimethylformamide (2×150 mL), dichloromethane (2×150 mL) and N,N-dimethylformamide (2×150 mL).

Fmoc group was removed by treatment with 20% piperidine in N,N-dimethylformamide (2×150 mL, 1×5 min, 1×20 min). Resin was washed with N,N-dimethylformamide (2×150 mL), 2-propanol (2×150 mL), dichloromethane (2×150 mL) and N,N-dimethylformamide (2×150 mL). Solution of Fmoc-Glu-OtBu (10.08 g, 23.69 mmol), O-(6-chloro-benzotriazol-1-yl)-N,N,N′,N′-tetramethyluronium tetrafluoroborate (TCTU, 8.42 g, 23.69 mmol) and N,N-diisopropylethylamine (7.43 mL, 42.64 mmol) in N,N-dimethylformamide (120 mL) was added to resin and mixture was shaken for 16 hr. Resin was filtered and treated with a solution of N,N-diisopropylethylamine (5.5 mL, 31.59 mmol) in methanol/dichloromethane mixture (9:1, 150 mL, 5 min). Then resin was washed with N,N-dimethylformamide (2×150 mL), dichloromethane (2×150 mL) and N,N-dimethylformamide (2×150 mL).

Fmoc group was removed by treatment with 20% piperidine in N,N-dimethylformamide (2×150 mL, 1×5 min, 1×20 min). Resin was washed with N,N-dimethylformamide (2×150 mL), 2-propanol (2×150 mL), dichloromethane (2×150 mL) and N,N-dimethylformamide (2×150 mL). Solution of Fmoc-Glu-OtBu (10.08 g, 23.69 mmol), O-(6-chloro-benzotriazol-1-yl)-N,N,N′,N′-tetramethyluronium tetrafluoroborate (TCTU, 8.42 g, 23.69 mmol) and N,N-diisopropylethylamine (7.43 mL, 42.64 mmol) in N,N-dimethylformamide (120 mL) was added to resin and mixture was shaken for 16 hr. Resin was filtered and treated with a solution of N,N-diisopropylethylamine (5.5 mL, 31.59 mmol) in methanol/dichloromethane mixture (9:1, 150 mL, 5 min). Then resin was washed with N,N-dimethylformamide (2×150 mL), dichloromethane (2×150 mL) and N,N-dimethylformamide (2×150 mL).

Fmoc group was removed by treatment with 20% piperidine in N,N-dimethylformamide (2×150 mL, 1×5 min, 1×20 min). Resin was washed with N,N-dimethylformamide (2×150 mL), 2-propanol (2×150 mL), dichloromethane (2×150 mL) and N,N-dimethylformamide (2×150 mL). Solution of tetradecanedioic acid mono-tert-butyl ester (7.45 g, 23.69 mmol), O-(6-chloro-benzotriazol-1-yl)-N,N,N′,N′-tetramethyluronium tetrafluoroborate (TCTU, 8.42 g, 23.69 mmol) and N,N-diisopropylethylamine (7.43 mL, 42.64 mmol) in the mixture of N,N-dimethylformamide (40 mL) and dichloromethane (80 mL) was added to resin and mixture was shaken for 16 hr. Resin was filtered and washed with dichloromethane (2×150 mL), N,N-dimethylformamide (2×150 mL), methanol (2×150 mL) and dichloromethane (10×150 mL).

The product was cleaved from the resin by the treatment with trifluoroethanol (150 mL) overnight. Resin was filtered off and washed with dichloromethane (3×100 mL). The solvent was removed under reduced pressure. The residue was purified by column chromatography on silica gel (gradient elution dichloromethane/methanol 100:0 to 95:5) giving titled compound as white solid.

Product was dried in vacuo to yield (S)-2-((S)-4-tert-Butoxycarbonyl-4-{(S)-4-tert-butoxycarbonyl-4-[(S)-4-tert-butoxycarbonyl-4-(13-tert-butoxycarbonyl-tridecanoylamino)-butyrylamino]-butyrylamino}-butyrylamino)-pentanedioic acid 1-tert-butyl ester.

Yield: 14.77 g (89%).

¹H NMR spectrum (300 MHz, CDCl₃, δH): 7.22 (d, 3=7.7 Hz, 1H); 6.97 (d, 3=7.9 Hz, 1H); 6.72 (d, 3=7.9 Hz, 1H); 6.41 (d, 3=7.9 Hz, 1H); 4.59-4.43 (m, 4H); 2.49-2.13 (m, 16H); 2.06-1.72 (m, 4H); 1.70-1.52 (m, 4H); 1.52-1.38 (m, 45H); 1.35-1.21 (m, 16H).

LC-MS purity: 100% (ELSD).

LC-MS Rt (Sunfire 4.6 mm×100 mm, acetonitrile/water 50:50 to 100:0+0.1% FA): 7.39 min.

LC-MS m/z: 1055.0 (M+H)+.

The obtained tert-butyl protected tetradecanedioyl-4×gGlu-OH ((S)-2-((S)-4-tert-Butoxycarbonyl-4-{(S)-4-tert-butoxycarbonyl-4-[(S)-4-tert-butoxycarbonyl-4-(13-tert-butoxycarbonyl-tridecanoylamino)-butyrylamino]-butyrylamino}-butyrylamino)-pentanedioic acid 1-tert-butyl ester) was dissolved in tetrahydrofuran. DIPEA was added followed by TSTU dissolved in acetonitrile. The reaction mixture was stirred for 3 h and then evaporated in vacuo, re-dissolved in ethyl acetate, washed with 0.1M HCl (aq), dried over MgSO₄, filtered and evaporated in vacuo. LC-MS (electrospray): m/z=1174.7 (M+Na⁺). Calc: 1175.4.

The protected and OSu-activated compound was dissolved in 10 mL TFA and stirred at room temperature overnight. Diethyl ether was added and the precipitate formed was filtered off and dried on vacuum overnight to afford (S)-2-((S)-4-Carboxy-4-{(S)-4-carboxy-4-[(S)-4-carboxy-4-(13-carboxy-tridecanoylamino)-butyrylamino]-butyrylamino}-butyrylamino)-pentanedioic acid 5-(2,5-dioxo-pyrrolidin-1-yl) ester (tetradecanedioyl-4×gGlu-OSu). LC-MS (electrospray): m/z=872.2 (M+H⁺). Calc: 871.9.

### General Procedure for Synthesis of Acylation Reagent on Solid Phase:

### Synthesis of tetradecanedioyl-gGlu-2×OEG-OSu (Chem. 5)

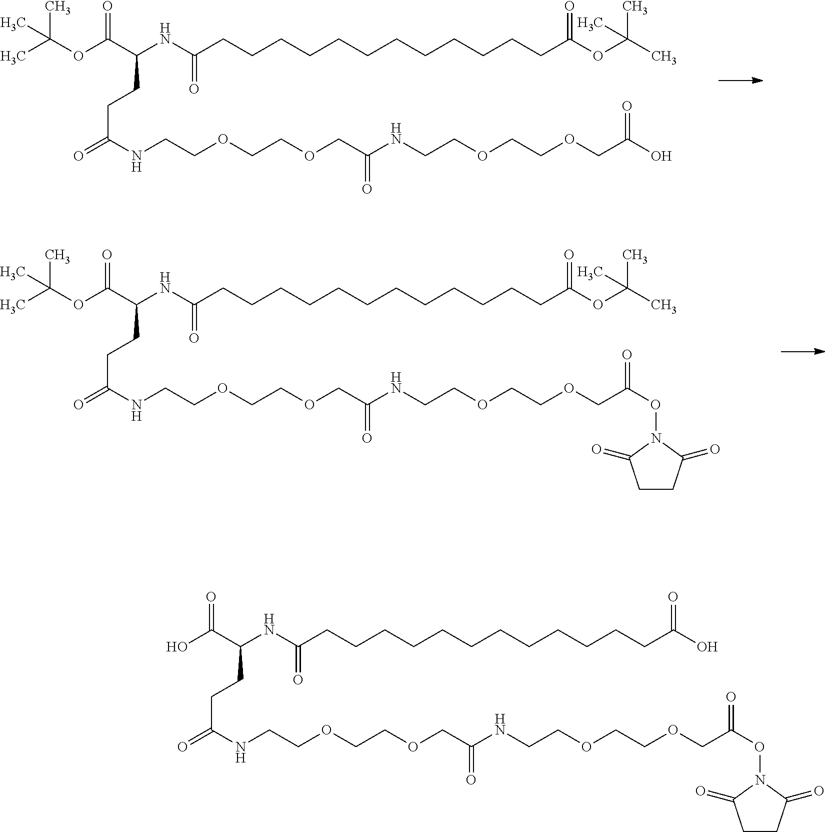

### 13-{(S)-1-tert-Butoxycarbonyl-3-[2-(2-{[2-(2-carboxymethoxy-ethoxy)-ethylcarbamoyl]-methoxy}-ethoxy)-ethylcarbamoyl]-propylcarbamoyl}-tridecanoic Acid tert-butyl Ester

2-Chlorotrityl resin 100-200 mesh 1.7 mmol/g (79.8 g, 135.6 mmol) was left to swell in dry dichloromethane (450 mL) for 20 minutes. A solution of {2-[2-(9H-fluoren-9-ylmethoxycarbonylamino)-ethoxy]-ethoxy}-acetic acid (Fmoc-OEG-OH, 34.9 g, 90.4 mmol) and N,N-diisopropylethylamine (59.9 mL, 343.6 mmol) in dry dichloromethane (100 mL) was added to resin and the mixture was shaken for 4 hrs. Resin was filtered and treated with a solution of N,N-diisopropylethylamine (31.5 mL, 180.8 mmol) in methanol/dichloromethane mixture (4:1, 150 mL, 2×5 min). Then resin was washed with N,N-dimethylformamide (2×300 mL), dichloromethane (2×300 mL) and N,N-dimethylformamide (3×300 mL). Fmoc group was removed by treatment with 20% piperidine in dimethylformamide (1×5 min, 1×30 min, 2×300 mL). Resin was washed with N,N-dimethylformamide (3×300 mL), 2-propanol (2×300 mL) and dichloromethane (350 mL, 2×300 mL).

Solution of {2-[2-(9H-fluoren-9-ylmethoxycarbonylamino)-ethoxy]-ethoxy}-acetic acid (Fmoc-OEG-OH, 52.3 g, 135.6 mmol), O-(6-chloro-benzotriazol-1-yl)-N,N,N′,N′-tetramethyluronium tetrafluoroborate (TCTU, 48.2 g, 135.6 mmol) and N,N-diisopropylethylamine (42.5 mL, 244.1 mmol) in N,N-dimethylformamide (250 mL) was added to resin and mixture was shaken for 2 hr. Since ninhydrin test was still positive, resin was filtered and treated with the same amounts of reagents for another 30 minutes. Resin was filtered and washed with N,N-dimethylformamide (2×300 mL), dichloromethane (2×300 mL) and N,N-dimethylformamide (3×300 mL). Fmoc group was removed by treatment with 20% piperidine in dimethylformamide (1×5 min, 1×30 min, 2×300 mL). Resin was washed with N,N-dimethylformamide (3×300 mL), 2-propanol (2×300 mL) and dichloromethane (350 mL, 2×300 mL).

Solution of (S)-2-(9H-fluoren-9-ylmethoxycarbonylamino)-pentanedioic acid 1-tert-butyl ester (Fmoc-LGlu-OtBu, 57.7 g, 135.6 mmol), O-(6-chloro-benzotriazol-1-yl)-N,N,N′,N′-tetramethyluronium tetrafluoroborate (TCTU, 48.2 g, 135.6 mmol) and N,N-diisopropylethylamine (42.5 mL, 244.1 mmol) in N,N-dimethylformamide (250 mL) was added to resin and mixture was shaken for 1 hr. Resin was filtered and washed with N,N-dimethylformamide (2×300 mL), dichloromethane (2×300 mL) and N,N-dimethylformamide (2×300 mL). Fmoc group was removed by treatment with 20% piperidine in dimethylformamide (1×5 min, 1×30 min, 2×300 mL). Resin was washed with N,N-dimethylformamide (3×300 mL), 2-propanol (2×300 mL) and dichloromethane (350 mL, 2×300 mL).

Solution of tetradecanedioic acid mono-tert-butyl ester (C14(OtBu)-OH, 42.7 g, 135.6 mmol), O-(6-chloro-benzotriazol-1-yl)-N,N,N′,N′-tetramethyluronium tetrafluoroborate (TCTU, 48.2 g, 135.6 mmol) and N,N-diisopropylethylamine (42.5 mL, 244.1 mmol) in dichloromethane/N,N-dimethylformamide mixture (4:1, 300 mL) was added to resin and mixture was shaken for 1.5 hr. Resin was filtered and washed with N,N-dimethylformamide (6×300 mL), dichloromethane (4×300 mL), methanol (4×300 mL) and dichloromethane (7×600 mL). The product was cleaved from resin by treatment with 2,2,2-trifluorethanol (600 mL) for 18 hrs. Resin was filtered off and washed with dichloromethane (4×300 mL), dichloromethane/2-propanol mixture (1:1, 4×300 mL), 2-propanol (2×300 mL) and dichloromethane (6×300 mL). Solutions were combined; solvent evaporated and crude product was purified by column chromatography (Silicagel 60A, 0.060-0.200 mm; eluent: dichloromethane/methanol 1:0-9:1).

Pure 13-{(S)-1-tert-Butoxycarbonyl-3-[2-(2-{[2-(2-carboxymethoxy-ethoxy)-ethylcarbamoyl]-methoxy}-ethoxy)-ethylcarbamoyl]-propylcarbamoyl}-tridecanoic acid tert-butyl ester was dried in vacuo and obtained as orange oil.

Yield: 55.2 g (77%).

RF (SiO₂, dichloromethane/methanol 9:1): 0.35.

1H NMR spectrum (300 MHz, CDCl₃, δH): 7.37 (bs, 1H); 7.02 (bs, 1H); 6.53 (d, J=7.9 Hz, 1H); 4.54-4.38 (m, 1H); 4.17 (s, 2H); 4.02 (s, 2H); 3.82-3.40 (m, 16H); 2.37-2.12 (m, 7H); 2.02-1.82 (m, 1H); 1.71-1.51 (m, 4H); 1.47 (s, 9H); 1.43 (s, 9H); 1.25 (bs, 16H).

LC-MS purity: 100%.

LC-MS Rt (Sunfire 4.6 mm×100 mm, acetonitrile/water 70:30 to 100:0+0.1% FA): 3.93 min.

LC-MS m/z: 791.0 (M+H)+.

13-{(S)-1-tert-Butoxycarbonyl-3-[2-(2-{[2-(2-carboxymethoxy-ethoxy)-ethylcarbamoyl]-methoxy}-ethoxy)-ethylcarbamoyl]-propylcarbamoyl}-tridecanoic acid tert-butyl ester (tetradecanedioyl-gGlu-2×OEG-OH, 8.89 g, 11.3 mmol)) was dissolved in 100 mL of acetonitrile, and TSTU (4.07 g, 13.5 mmol) and DIPEA (2.35 mL, 13.5 mmol) were added to the stirred solution and the mixture was stirred at room temperature for 1 hour. The solvent was evaporated and the residue was dissolved in dichloromethane and washed twice with 0.05M HCl.

The organic phase was dried (MgSO₄) and evaporated in vacuo. This afforded 9.98 g (100%) of 13-((S)-1-tert-Butoxycarbonyl-3-{2-[2-({2-[2-(2,5-dioxo-pyrrolidin-1-yloxycarbonylmethoxy)-ethoxy]-ethylcarbamoyl}-methoxy)-ethoxy]-ethylcarbamoyl}-propylcarbamoyl)-tridecanoic acid tert-butyl ester as an oil.

13-((S)-1-tert-Butoxycarbonyl-3-{2-[2-({2-[2-(2,5-dioxo-pyrrolidin-1-yloxycarbonyl methoxy)-ethoxy]-ethylcarbamoyl}-methoxy)-ethoxy]-ethylcarbamoyl}-propylcarbamoyl)-tridecanoic acid tert-butyl ester (4 g) was dissolved in trifluoroacetic acid (10 mL) and the mixture was stirred at room temperature for 1 hour and evaporated in vacuo. The residue was dissolved in dichloromethane (10 mL) and evaporated in vacuo. Addition of cold diethyl ether (10 mL) resulted in precipitation of a white greasy solid. This was isolated by decantation and was dried in vacuo. This afforded 3.4 g (quant.) of 14-[[(1S)-1-carboxy-4-[2-[2-[2-[2-[2-[2-(2,5-dioxopyrrolidin-1-yl)oxy-2-oxoethoxy]ethoxy]ethylamino]-2-oxoethoxy]ethoxy]ethylamino]-4-oxobutyl]amino]-14-oxotetradecanoic acid (tetradecanedioyl-gGlu-2×OEG-OSu), which was stored at −18° C.

LC-MS (electrospray): m/z=775.33; calc: 774.8.

### General Procedure (A) for Acylation of Insulins and Purification of Acylated Analogues

A general procedure (A) for the acylation and purification of the insulin derivatives of the invention is described in detail in Example 1, below, and has been applied to the synthesis of additional compounds as indicated below. Purification using other methods (as described above) has also been done for some of these derivatives.

Acylated analogues of the invention are made by acylation of recombinant insulin analogues by acylation in an aqueous environment at high pH such as pH 9.5, 10, 10.5 11, 11.5, 12, 12.5, or 13. The acylation reagent may be dissolved in water or in a non-aqueous polar solvent, such as DMF or NMP, and added to the insulin solution with vigorous stirring. After addition of the acylation reagent, conversion is analysed by HPLC, and if necessary, more acylation reagent is added. Purification is done as described above.

### General Procedure (B) for Solid Phase Synthesis and Purification of Acylated Analogues

A general procedure (B) for the solid phase synthesis and purification of the insulin derivatives of the invention is described below, and has been applied to the synthesis of additional compounds as indicated below. Purification using other methods (as described above) has also been done for some of these derivatives.

Insulin A and B chains were prepared on a Prelude peptide synthesiser using a general Fmoc based solid phase peptide coupling method.

### Resins Used

Fmoc-Lys(Mtt)-Wang; Fmoc-Ala-Wang; Fmoc-Gly-Wang, and Fmoc-Asp-OtBu coupled to PAL resin.

Amino acids (listed below) and oxyma (ethyl (hydroxyimino)cyanoacetate) were dissolved in DMF to a concentration of 0.3M: Fmoc-Ala-OH; Fmoc-Arg(Pbf)-OH; Fmoc-Asn(Trt)-OH; Fmoc-Asp(OtBu)-OH; Fmoc-Cys(Trt)-OH; Fmoc-Gln(Trt)-OH; Fmoc-Glu(OtBu)-OH; Fmoc-Gly-OH; Fmoc-His(Trt)-OH; Fmoc-Ile-OH; Fmoc-Leu-OH; Fmoc-Lys(Boc)-OH; Fmoc-Met-OH; Fmoc-Phe-OH; Fmoc-Pro-OH; Fmoc-Ser(tBu)-OH; Fmoc-Thr(tBu)-OH; Fmoc-Trp(Boc)-OH; Fmoc-Tyr(tBu)-OH; and Fmoc-Val-OH.

Special/unnatural amino acids: Boc-Phe-OH; Boc-Gly-OH; and Fmoc-Cys(Acm)-OH.

### Procedure

Standard coupling conditions used on resins were: 8 eq amino acid, DIC, collidine and oxyma in NMP for 1 hour, in the case of Fmoc-Arg(Pbf)-OH, a double coupling protocol (2×1 h) was used.

Standard deprotection conditions used were: 20% piperdine in NMP (2×5.5 mL for 2×7.5 min or 2×10 min), followed by washing with NMP and DCM.

For acylation at Lys prior to cleavage from the resin the following protocol is used (in this case the N-terminal AA is Boc protected)

### Deprotection of Mtt Group and Acylation with tBu-Protected Activated Acylation Reagent ([Acyl]-[Linker]-OSu, eg, tetradecanedioyl-gGlu-2×OEG-OSu and tetradecanedioyl-gGlu-2×OEG-OSu (both Protected as tBu Esters at Terminal and Alpha Carboxyl Groups)

Step 1:

To the resin was added HFIP (12 mL), and the reaction shaken for 20 min. After removal of solvent by filtration the resin was washed with DCM (4×15 mL) and dried over a nitrogen stream.

Step 2:

To the above resin was added DMF (8 mL) and DIPEA (1.5 mL). A solution of activated acylation reagent (0.75 g in 2 mL DMF) was then added and the reaction shaken for 15 h, drained and washed with DCM (3×15 mL).

Alternatively, the side chain can be built sequentially.

### Deprotection of the Mtt Group

To the resin was added HFIP (6 mL), and the reaction incubated for 20 min. After removal of the solvent the resin was washed with DCM (6 mL). HFIP (6 mL) was added to the resin, and the reaction incubated for 20 min. The resin was washed with DCM (2×7.5 mL) and Collidine (2×7.5 mL), followed by additional washes with DCM (2×7.5 mL).

The side chain was built up by sequential standard couplings using Fmoc-Glu-OtBu, Fmoc-OEG-OH, and 14-tert-butoxy-14-oxo-tetradecanoic acid or 16-tert-butoxy-16-oxo-hexadecanoic acid.

### A6C-A11C Disulfide Formation

The resin was treated for 15 min with a 0.5% solution of iodine in DCM/HFIP (30 mL of 1:1 mixture). After removal of solvent by filtration the resin was washed with DCM (3×20 mL) and dried over a nitrogen stream.

A-Chain Cleavage from the Resin and Activation of A20-Cys as S—S-Pyridyl

The resin was treated with a solution of TFA (30 mL), triisopropylsilane (1 mL), water (0.75 mL) and dithiodipyridine (0.75 g) for 3 h, and then the filtrate was collected and added to 150 mL diethyl ether (split into 6 plastic NUNC tubes) to precipitate the peptide. The tubes were centrifuged at 3500 rpm for 3 min, the ether layer was decanted, and this ether step was repeated a further 3 times. The crude material was combined and allowed to dry overnight at RT to give the desired peptide A-chain.

B-Chain Cleavage from the Resin

The resin was treated with a solution of TFA (30 mL), triisopropylsilane (1 mL), water (0.75 mL) and dithiothreitol (0.5 g) for 3 h, and then the filtrate was collected and added to diethyl ether (150 mL, split into 6 plastic NUNC tubes) to precipitate the peptide. The tubes were centrifuged at 3500 rpm for 3 min, the ether layer was decanted, and this ether step was repeated a further 3 times. The crude material was allowed to dry overnight at RT to give the desired peptide B-chain.

### A20C-B19C Disulfide Formation

To a mixture of A-chain (0.33 g) and B-chain (0.33 g) was added DMSO (8 mL) and DIPEA (1 mL) and the mixture stirred for 20 min, then added dropwise with stirring to 140 mL of neutral buffer solution (water, TRIS (10 mM), ammonium sulfate (15 mM), 20% acetonitrile) to a total volume of ca 150 mL.

The mixture was then purified by reverse phase chromatography using following set up:
Phenomenex Gemini 5 μM 5 u C18 110 Å 30×250 mm column, running at 20 mL/min 10% B to 60% B over 40 min
Eluant A=10 mM TRIS, 15 mM ammonium sulfate, pH=7.3, 20% ACN in milliQ water
Eluant B=20% milliQ water in acetonitrile

Pure fractions were pooled, flash frozen and freeze dried.

### A7C-B7C Disulfide Formation

Freeze dried intermediate from the previous step was redissolved in 5 mL DMSO. Acetic acid (20 mL) and water (4 mL) was added, followed by iodine in AcOH (3 mL of 40 mM)

After total reaction time of 20 min, the reaction quenched with 1M sodium ascorbate, and then added to a stirred solution of water (90 mL).

The mixture was then purified by reverse phase chromatography using following set up:
Phenomenex Gemini 5 μM 5 u C18 110 Å 30×250 mm column, running at 20 mL/min 10% B to 45% B over 40 min
Eluant A=0.1% TFA in milliQ water
Eluant B=0.1% TFA in acetonitrile

Pure fractions were pooled, flash frozen and freeze dried to give the desired product.

### Example 1

### General Procedure (A)

### A21G, B3E, B28D, B29K(N(eps)tetradecanedioyl-gGlu-2×OEG), desB30 Human Insulin; (SEQ ID NOS: 7 and 13)

IUPAC (OpenEye, IUPAC style) name: N{Epsilon-B29}-[2-[2-[2-[[2-[2-[2-[[(4S)-4-carboxy-4-(13-carboxytridecanoylamino)butanoyl]amino]ethoxy]ethoxy]acetyl]amino]-ethoxy]ethoxy]acetyl]-[GlyA21,GluB3,AspB28],des-ThrB30-insulin(human).

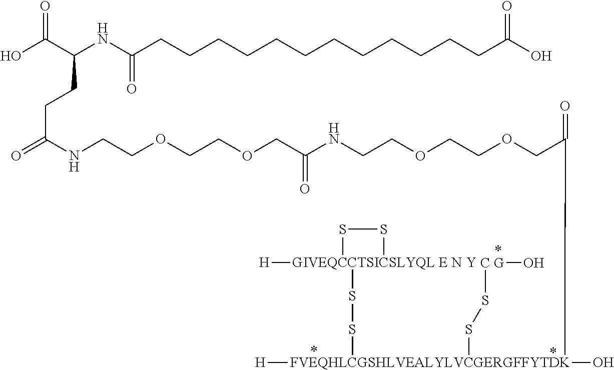

A21G, B3E, B28D, desB30 human insulin (0.68 g, 0.12 mmol) was dissolved in 10 ml 100 mM aqueous Na₂CO₃, and pH was adjusted to 11.5 with 1M NaOH. 14-[[(1S)-1-Carboxy-4-[2-[2-[2-[2-[2-[2-(2,5-dioxopyrrolidin-1-yl)oxy-2-oxoethoxy]ethoxy]ethylamino]-2-oxoethoxy]ethoxy]ethylamino]-4-oxobutyl]amino]-14-oxotetradecanoic acid (tetradecanedioyl-gGlu-2×OEG-OSu) (0.23 g, 0.3 mmol) was dissolved in 1 ml NMP and added drop wise with vigorous stirring while keeping pH at 12.0 to 10.8 with addition of 1N NaOH. More tetradecanedioyl-gGlu-2×OEG-OSu (0.11 g, dissolved in 1 ml NMP) was added. pH was then adjusted to 1 with 1N HCl and acetonitrile (2 ml) was added. The mixture was purified by preparative HPLC (column: Phenomenex Gemini, 5 μM 5 u C18, 110 Å, 30×250 mm) using a gradient of 10% B to 40% B in 50 minutes, 20 ml/min. A-buffer: 0.1% TFA in water, B-buffer: 0.1% TFA in acetonitrile. Pure fractions were pooled and lyophilised to afford 0.245 g (32%) of the title insulin.

LC-MS (electrospray): m/z=1586.4 (M+4)/4. Calc: 1586.6.

### Example 2

### General Procedure (A)

### A21G, B3E, B27E, B28D, B29K(N(eps)tetradecanedioyl-gGlu-2×OEG), desB30 Human Insulin; (SEQ ID NOS: 7 and 11)

IUPAC (OpenEye, IUPAC style) name: N{Epsilon-B29}-[2-[2-[2-[[2-[2-[2-[[(4S)-4-carboxy-4-(13-carboxytridecanoylamino)butanoyl]amino]ethoxy]ethoxy]acetyl]amino]ethoxy]ethoxy]acetyl]-[GlyA21,GluB3,GluB27,AspB28],des-ThrB30-Insulin(human).

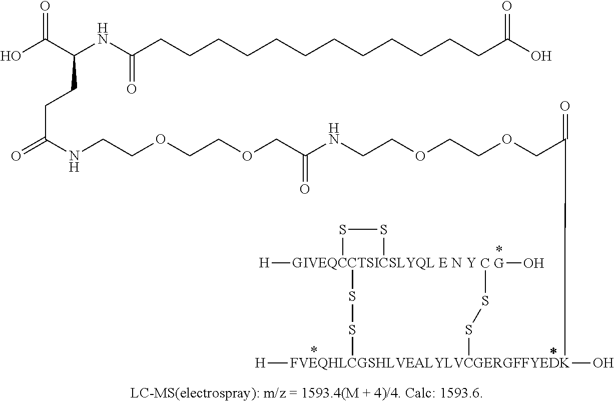

### Example 3

### General Procedure (A)

### A21G, B3E, B27E, B28E, B29K(N(eps)tetradecanedioyl-gGlu-2×OEG), desB30 Human Insulin; (SEQ ID NOS: 7 and 12)

IUPAC (OpenEye, IUPAC style) name: N{Epsilon-B29}-[2-[2-[2-[[2-[2-[2-[[(4S)-4-carboxy-4-(13-carboxytridecanoylamino)butanoyl]amino]ethoxy]ethoxy]acetyl]amino]ethoxy]ethoxy]acetyl]-[GlyA21,GluB3,GluB27,GluB28],des-ThrB30-insulin(human).

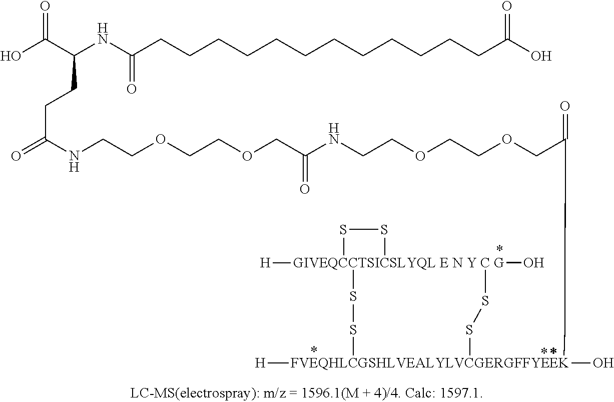

### Example 4

### General Procedure (A)

### B3E, B28D, B29K(N(eps)tetradecanedioyl-gGlu-2×OEG), desB30 Human Insulin; (SEQ ID NO:13)

IUPAC (OpenEye, IUPAC style) name: N{Epsilon-B29}-[2-[2-[2-[[2-[2-[2-[[(4S)-4-carboxy-4-(13-carboxytridecanoylamino)butanoyl]amino]ethoxy]ethoxy]acetyl]amino]ethoxy]ethoxy]acetyl]-[GluB3,AspB28],des-ThrB30-Insulin(human).

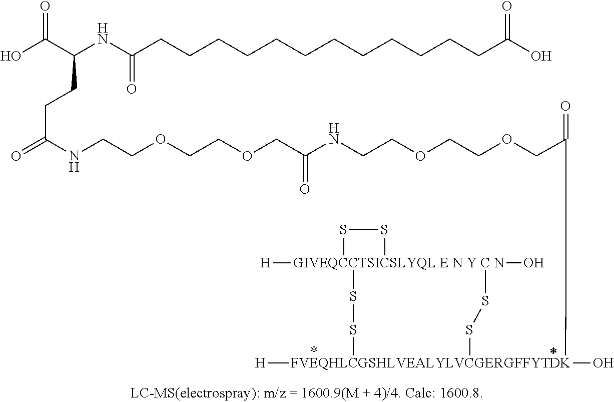

### Example 5

### General Procedure (A)

### A8H, A21A, B3E, B28D, B29K(N(eps)tetradecanedioyl-gGlu-2×OEG), desB30 Human Insulin; (SEQ ID NOS: 2 and 13)

IUPAC (OpenEye, IUPAC style) name: N{Epsilon-B29}-[2-[2-[2-[[2-[2-[2-[[(4S)-4-carboxy-4-(13-carboxytridecanoylamino)butanoyl]amino]ethoxy]ethoxy]acetyl]amino]ethoxy]ethoxy]acetyl]-[HisA8,AlaA21,GluB3,AspB28],des-ThrB30-Insulin(human).

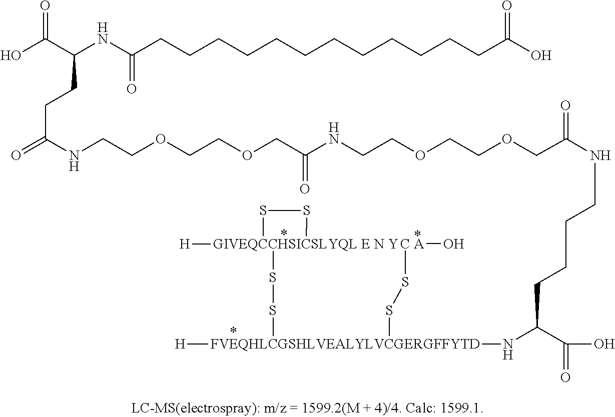

### Example 6

### General Procedure (B)

### A8H, A21G, B3E, B28D, B29K(N(eps)tetradecanedioyl-gGlu-2×OEG), desB30 Human Insulin; (SEQ ID NOS: 3 and 13)

IUPAC (OpenEye, IUPAC style) name: N{Epsilon-B29}-[2-[2-[2-[[2-[2-[2-[[(4S)-4-carboxy-4-(13-carboxytridecanoylamino)butanoyl]amino]ethoxy]ethoxy]acetyl]amino]ethoxy]ethoxy]acetyl]-[HisA8,GlyA21,GluB3,AspB28],des-ThrB30-Insulin(human).

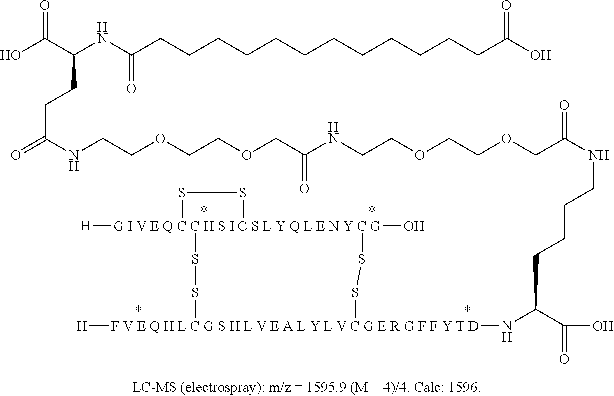

### Example 7

### General Procedure (B)

### A8H, B3E, B27E, B28E, B29K(N(eps)tetradecanedioyl-gGlu-2×OEG), desB30 Human Insulin; (SEQ ID NOS: 1 and 12)

IUPAC (OpenEye, IUPAC style) name: N{Epsilon-B29}-[2-[2-[2-[[2-[2-[2-[[(4S)-4-carboxy-4-(13-carboxytridecanoylamino)butanoyl]amino]ethoxy]ethoxy]acetyl]amino]ethoxy]ethoxy]acetyl]-[HisA8,GluB3,GluB27,GluB28],des-ThrB30-Insulin(human).

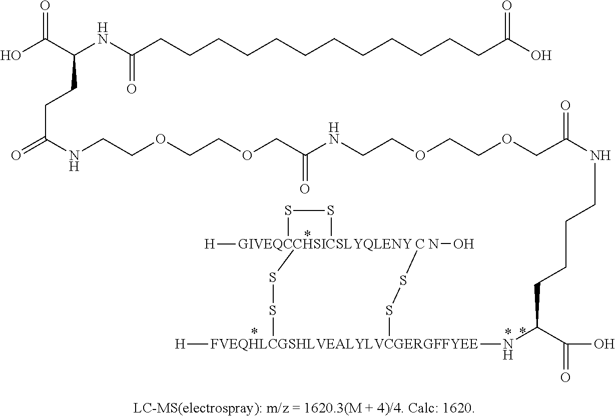

### Example 8

### General Procedure (B)

### A8H, A21A, B3E, B27E, B28E, B29K(N(eps)tetradecanedioyl-gGlu-2×OEG), desB30 Human Insulin; (SEQ ID NOS: 2 and 12)

IUPAC (OpenEye, IUPAC style) name: N{Epsilon-B29}-[2-[2-[2-[[2-[2-[2-[[(4S)-4-carboxy-4-(13-carboxytridecanoylamino)butanoyl]amino]ethoxy]ethoxy]acetyl]amino]ethoxy]ethoxy]acetyl]-[HisA8,AlaA21,GluB3,GluB27,GluB28],des-ThrB30-insulin(human).

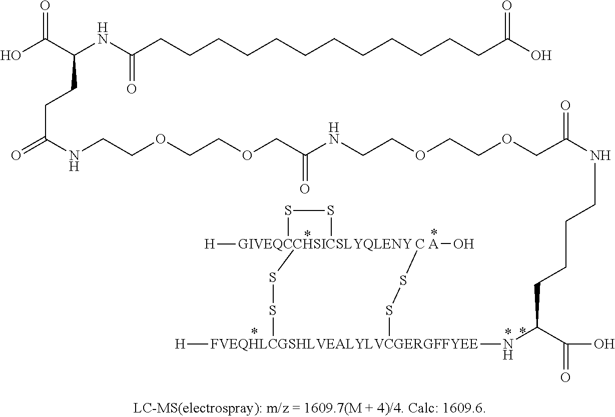

### Example 9

### May be Prepared According to General Procedure (A or B)

### A8H, A21G, B3E, B27E, B28E, B29K(N(eps)tetradecanedioyl-gGlu-2×OEG), desB30 Human Insulin; (SEQ ID NOS: 3 and 12)

IUPAC (OpenEye, IUPAC style) name: N{Epsilon-B29}-[2-[2-[2-[[2-[2-[2-[[(4S)-4-carboxy-4-(13-carboxytridecanoylamino)butanoyl]amino]ethoxy]ethoxy]acetyl]amino]ethoxy]ethoxy]acetyl]-[HisA8,GlyA21,GluB3,GluB27,GluB28],des-ThrB30-insulin(human).

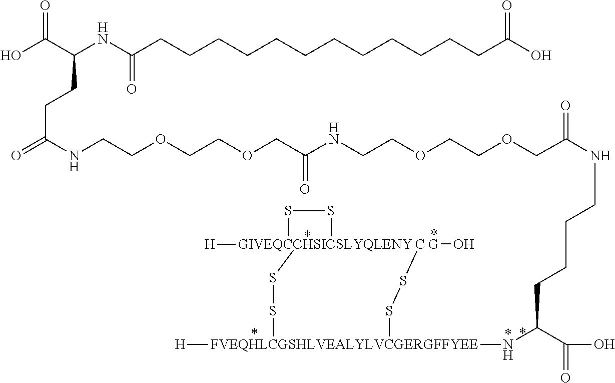

### Example 10

### General Procedure (B)

### B3E, B27E, B28E, B29K(N(eps)tetradecanedioyl-gGlu-2×OEG), desB30 Human Insulin; (SEQ ID NO:12)

IUPAC (OpenEye, IUPAC style) name: N{Epsilon-B29}-[2-[2-[2-[[2-[2-[2-[[(4S)-4-carboxy-4-(13-carboxytridecanoylamino)butanoyl]amino]ethoxy]ethoxy]acetyl]amino]ethoxy]ethoxy]acetyl]-[GluB3,GluB27,GluB28],des-ThrB30-Insulin(human).

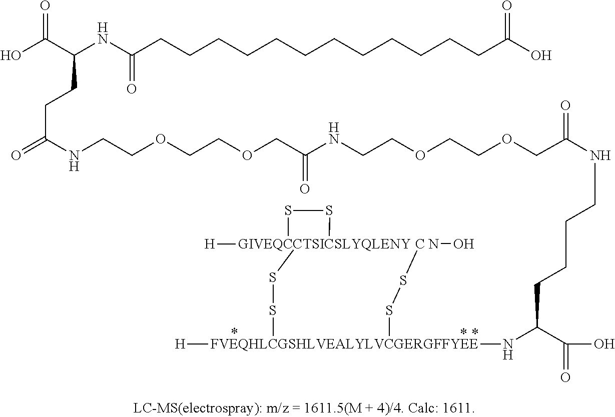

### Example 11

### General Procedure (B)

### A21A, B3E, B27E, B28E, B29K(N(eps)tetradecanedioyl-gGlu-2×OEG), desB30 Human Insulin; (SEQ ID NOS: 6 and 12)

IUPAC (OpenEye, IUPAC style) name: N{Epsilon-B29}-[2-[2-[2-[[2-[2-[2-[[(4S)-4-carboxy-4-(13-carboxytridecanoylamino)butanoyl]amino]ethoxy]ethoxy]acetyl]amino]ethoxy]ethoxy]acetyl]-[AlaA21,GluB3,GluB27,GluB28],des-ThrB30-Insulin(human).

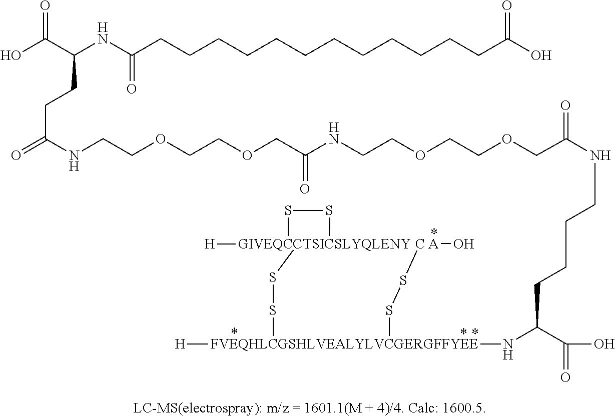

### Example 12

### General Procedure (B)

### A8H, B3E, B28D, B29K(N(eps)tetradecanedioyl-gGlu-2×OEG), desB30 Human Insulin; (SEQ ID NOS: 1 and 13)

IUPAC (OpenEye, IUPAC style) name: N{Epsilon-B29}-[2-[2-[2-[[2-[2-[2-[[(4S)-4-carboxy-4-(13-carboxytridecanoylamino)butanoyl]amino]ethoxy]ethoxy]acetyl]amino]ethoxy]ethoxy]acetyl]-[HisA8,GluB3,AspB28],des-ThrB30-Insulin(human).

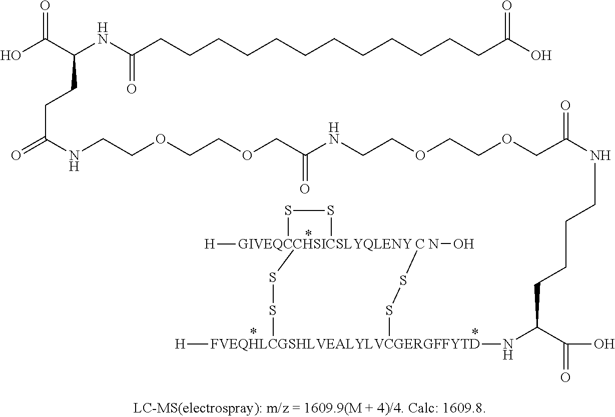

### Example 13

### General Procedure (A and B)

### A21A, B3E, B28D, B29K(N(eps)tetradecanedioyl-gGlu-2×OEG), desB30 Human Insulin; (SEQ ID NOS: 6 and 13)

IUPAC (OpenEye, IUPAC style) name: N{Epsilon-B29}-[2-[2-[2-[[2-[2-[2-[[(4S)-4-carboxy-4-(13-carboxytridecanoylamino)butanoyl]amino]ethoxy]ethoxy]acetyl]amino]ethoxy]ethoxy]acetyl]-[AlaA21,GluB3,AspB28],des-ThrB30-Insulin(human).

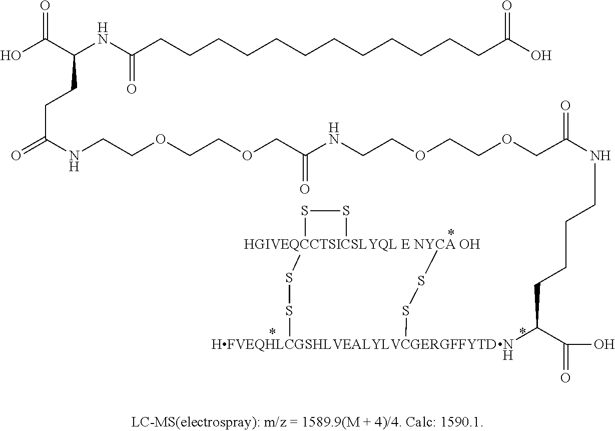

### Example 14

May be Prepared according to General Procedure (A or B)

### B3Q, B28D, B29K(N(eps)tetradecanedioyl-gGlu-2×OEG), desB30 Human Insulin; (SEQ ID NO:16)

IUPAC (OpenEye, IUPAC style) name: N{Epsilon-B29}-[2-[2-[2-[[2-[2-[2-[[(4S)-4-carboxy-4-(13-carboxytridecanoylamino)butanoyl]amino]ethoxy]ethoxy]acetyl]amino]ethoxy]ethoxy]acetyl]-[GlnB3,AspB28],des-ThrB30-Insulin(human).

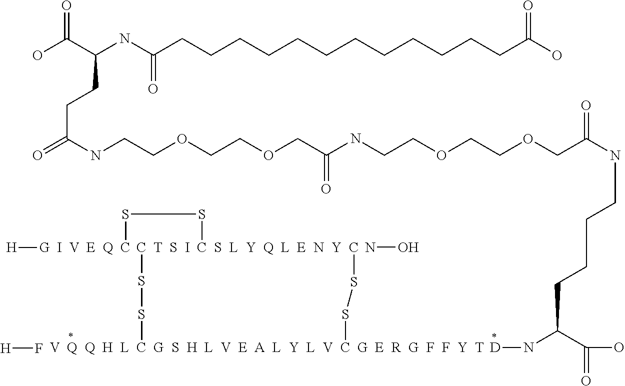

### Example 15

### May be Prepared According to General Procedure (A or B)

### A21A, B3Q, B28D, B29K(N(eps)tetradecanedioyl-gGlu-2×OEG), desB30 Human Insulin; (SEQ ID NOS: 6 and 16)

IUPAC (OpenEye, IUPAC style) name: N{Epsilon-B29}-[2-[2-[2-[[2-[2-[2-[[(4S)-4-carboxy-4-(13-carboxytridecanoylamino)butanoyl]amino]ethoxy]ethoxy]acetyl]amino]ethoxy]ethoxy]acetyl]-[AlaA21,GlnB3,AspB28],des-ThrB30-Insulin(human).

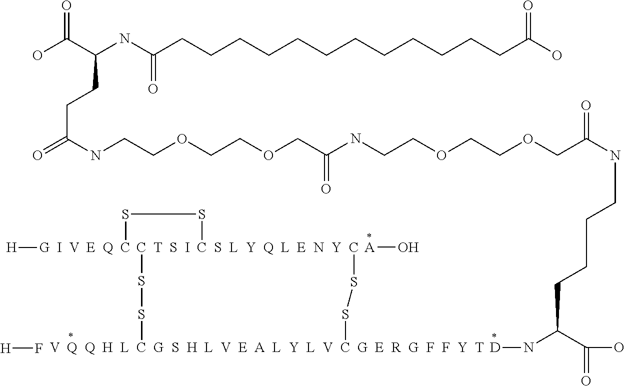

### Example 16

### General Procedure (A)

### A21G, B3E, B28D, B29K(N(eps)tetradecanedioyl-4×gGlu), desB30 Human Insulin; (SEQ ID NOS: 7 and 13)

IUPAC (OpenEye, IUPAC style) name: N{Epsilon-B29}-[(4S)-4-carboxy-4-[[(4S)-4-carboxy-4-[[(4S)-4-carboxy-4-[[(4S)-4-carboxy-4-(13-carboxytridecanoylamino) butanoyl]amino]butanoyl]amino]butanoyl]amino]butanoyl]-[GlyA21,GluB3,AspB28],des-ThrB30-Insulin(human).

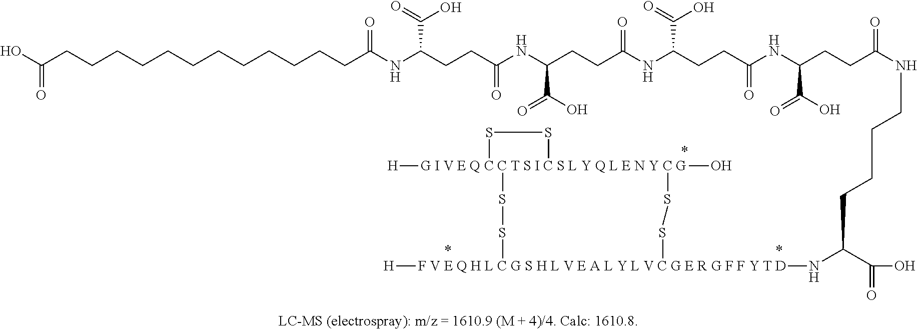

### Example 17

### General Procedure (A)

### B3E, B28D, B29K(N(eps)tetradecanedioyl-4×gGlu), desB30 Human Insulin; (SEQ ID NO:13)

IUPAC (OpenEye, IUPAC style) name: N{Epsilon-B29}-[(4S)-4-carboxy-4-[[(4S)-4-carboxy-4-[[(4S)-4-carboxy-4-[[(4S)-4-carboxy-4-(13-carboxytridecanoylamino) butanoyl]amino]butanoyl]amino]butanoyl]amino]butanoyl]-[GluB3,AspB28],des-ThrB30-Insulin(human).

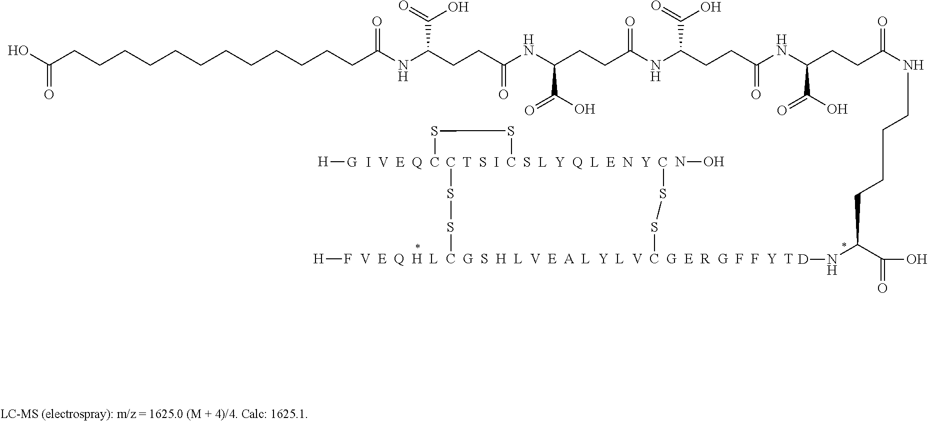

### Example 18

### General Procedure (B)

### A14E, A21A, B30, B28D, B29K(N(eps)tetradecanedioyl-4×gGlu), desB30 Human Insulin; (SEQ ID NOS: 5 and 16)

IUPAC (OpenEye, IUPAC style) name: N{Epsilon-B29}-[(4S)-4-carboxy-4-[[(4S)-4-carboxy-4-[[(4S)-4-carboxy-4-[[(4S)-4-carboxy-4-(13-carboxytridecanoylamino) butanoyl]amino]butanoyl]amino]butanoyl]amino]butanoyl]-[GluA14,AlaA21,GlnB3,AspB28],des-ThrB30-Insulin(human).

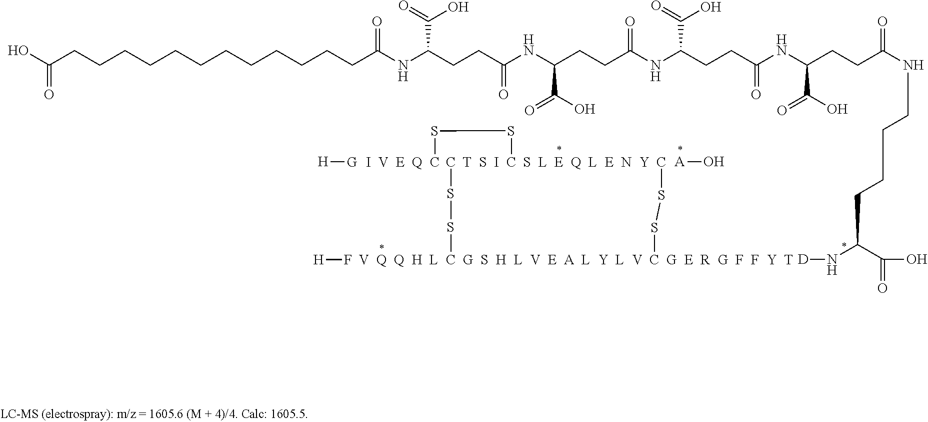

### Example 19

### General Procedure (A and B)

### A21A, B3E, B28E, B29K(N(eps)tetradecanedioyl-4×gGlu), desB30 Human Insulin; (SEQ ID NOS: 6 and 14)

IUPAC (OpenEye, IUPAC style) name: N{Epsilon-B29}-[(4S)-4-carboxy-4-[[(4S)-4-carboxy-4-[[(4S)-4-carboxy-4-[[(4S)-4-carboxy-4-(13-carboxytridecanoylamino)-butanoyl]amino]butanoyl]amino]butanoyl]amino]butanoyl]-[AlaA21,GluB3,GluB28],des-ThrB30-Insulin(human).

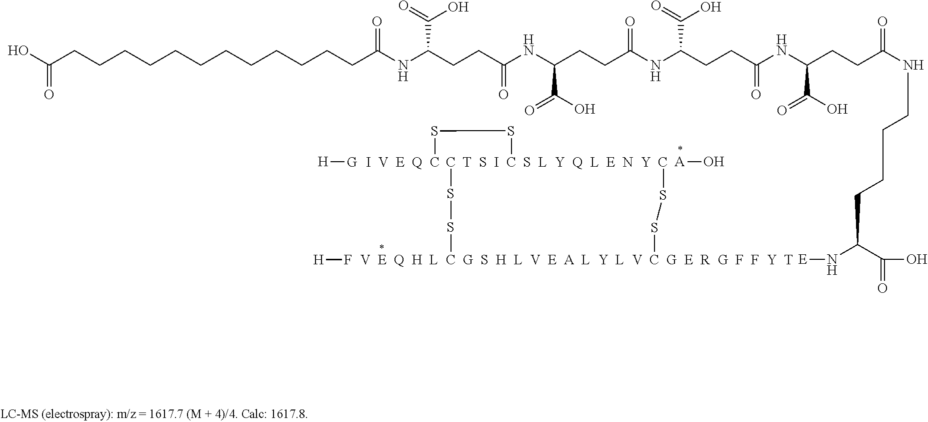

### Example 20

### General Procedure (A)

### A21A, B3E, B28D, B29K(N(eps)tetradecanedioyl-4×gGlu), desB30 Human Insulin; (SEQ ID NOS: 6 and 13)

IUPAC (OpenEye, IUPAC style) name: N{Epsilon-B29}-[(4S)-4-carboxy-4-[[(4S)-4-carboxy-4-[[(4S)-4-carboxy-4-[[(4S)-4-carboxy-4-(13-carboxytridecanoylamino) butanoyl]amino]butanoyl]amino]butanoyl]amino]butanoyl]-[AlaA21,GluB3,AspB28],des-ThrB30-Insulin(human).

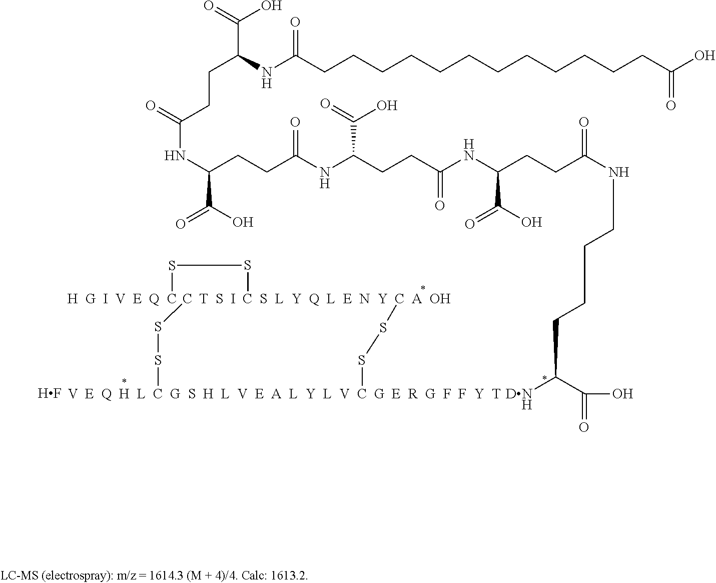

### Example 21

### General Procedure (A)

### A21A, B3E, B27E, B28E, B29K(N(eps)tetradecanedioyl-4×gGlu), desB30 Human Insulin; (SEQ ID NOS: 6 and 12)

IUPAC (OpenEye, IUPAC style) name: N{Epsilon-B29}-[(4S)-4-carboxy-4-[[(4S)-4-carboxy-4-[[(4S)-4-carboxy-4-[[(4S)-4-carboxy-4-(13-carboxytridecanoylamino)-butanoyl]amino]butanoyl]amino]butanoyl]amino]butanoyl]-[AlaA21,GluB3,GluB27,GluB28],des-ThrB30-Insulin(human).

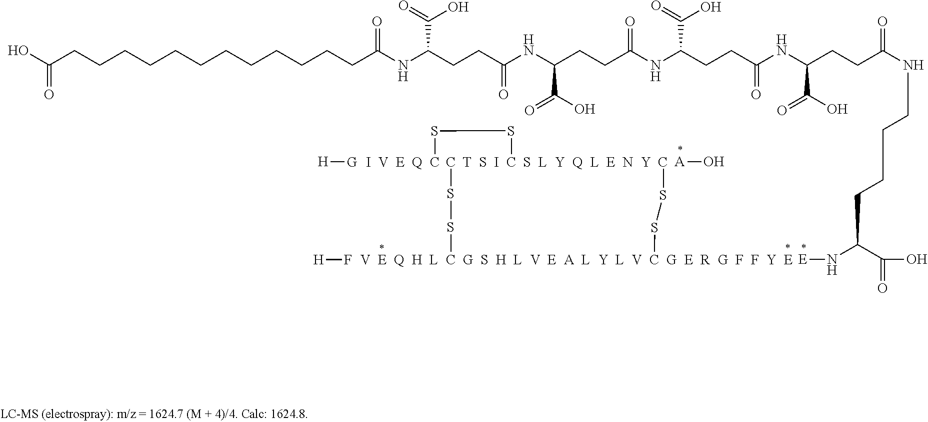

### Example 22

### General Procedure (B)

### A14E, B3Q, B28D, B29K(N(eps)tetradecanedioyl-4×gGlu), desB30 Human Insulin; (SEQ ID NOS: 4 and 16)

IUPAC (OpenEye, IUPAC style) name: N{Epsilon-B29}-[(4S)-4-carboxy-4-[[(4S)-4-carboxy-4-[[(4S)-4-carboxy-4-[[(4S)-4-carboxy-4-(13-carboxytridecanoylamino) butanoyl]amino]butanoyl]amino]butanoyl]amino]butanoyl]-[GluA14,GlnB3,AspB28],des-ThrB30-Insulin(human).

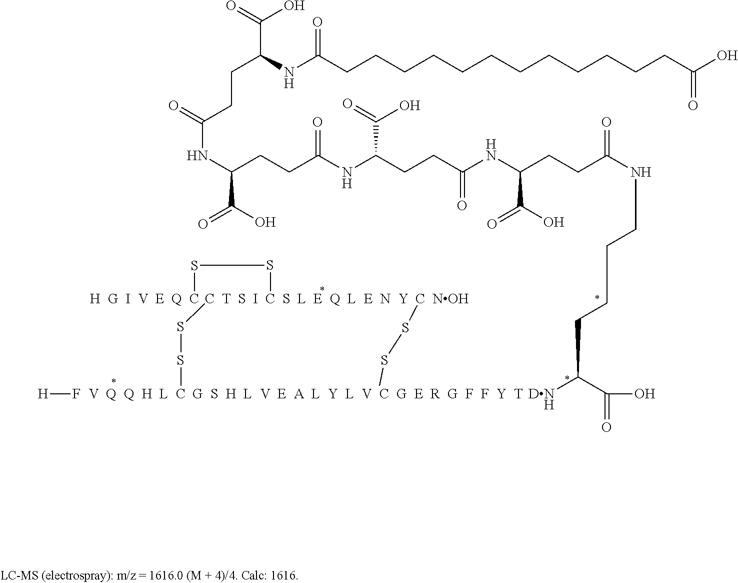

### Example 23

### General Procedure (A)

### B3E, B27E, B28E, B29K(N(eps)tetradecanedioyl-4×gGlu), desB30 Human Insulin; (SEQ ID NO:12)

IUPAC (OpenEye, IUPAC style) name: N{Epsilon-B29}-[(4S)-4-carboxy-4-[[(4S)-4-carboxy-4-[[(4S)-4-carboxy-4-[[(4S)-4-carboxy-4-(13-carboxytridecanoylamino)-butanoyl]amino]butanoyl]amino]butanoyl]amino]butanoyl]-[GluB3,GluB27,GluB28],des-ThrB30-Insulin(human).

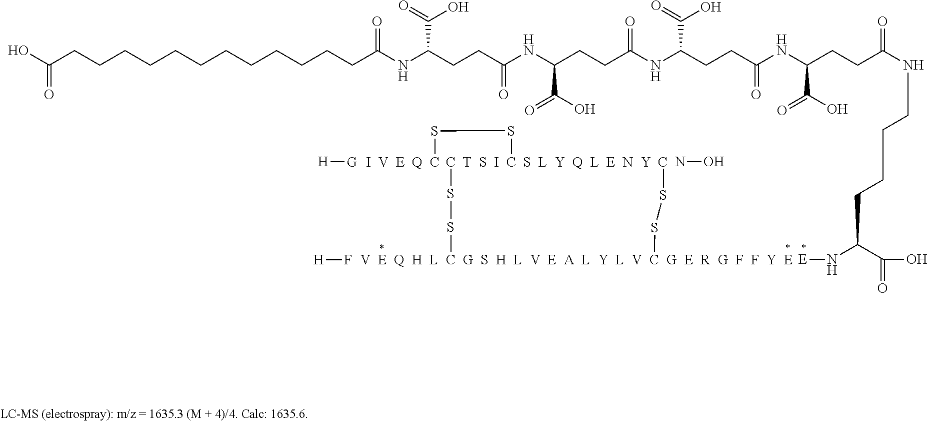

### Example 24

### General Procedure (A and B)

### B3Q, B28D, B29K(N(eps)tetradecanedioyl-4×gGlu), desB30 Human Insulin; (SEQ ID NO:16)

IUPAC (OpenEye, IUPAC style) name: N{Epsilon-B29}-[(4S)-4-carboxy-4-[[(4S)-4-carboxy-4-[[(4S)-4-carboxy-4-[[(4S)-4-carboxy-4-(13-carboxytridecanoylamino) butanoyl]amino]butanoyl]amino]butanoyl]amino]butanoyl]-[GlnB3,AspB28],des-ThrB30-Insulin(human).

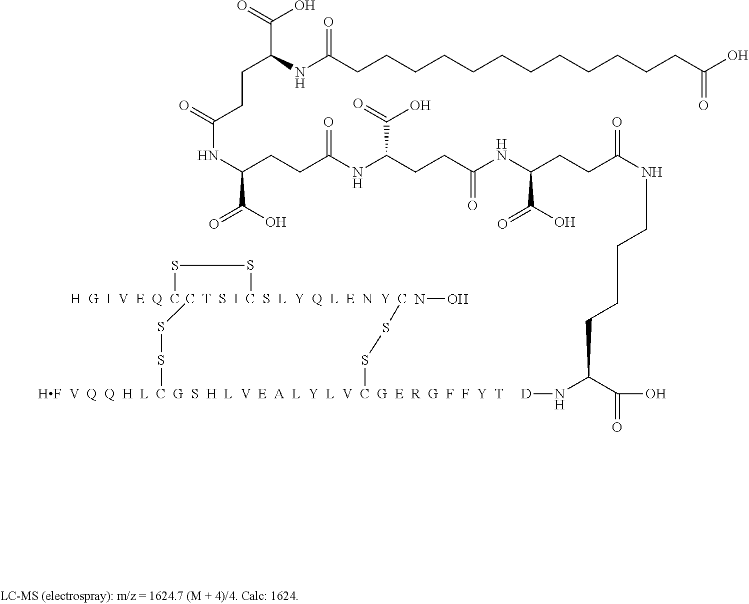

### Example 25

### General Procedure (A)

### A21G, B3E, B27E, B29K(N(eps)tetradecanedioyl-4×gGlu), desB30 Human Insulin; (SEQ ID NOS: 7 and 25)

IUPAC (OpenEye, IUPAC style) name: N{Epsilon-B29}-[(4S)-4-carboxy-4-[[(4S)-4-carboxy-4-[[(4S)-4-carboxy-4-[[(4S)-4-carboxy-4-(13-carboxytridecanoylamino)-butanoyl]amino]butanoyl]amino]butanoyl]amino]butanoyl]-[GlyA21,GluB3,GluB27],des-ThrB30-Insulin(human).

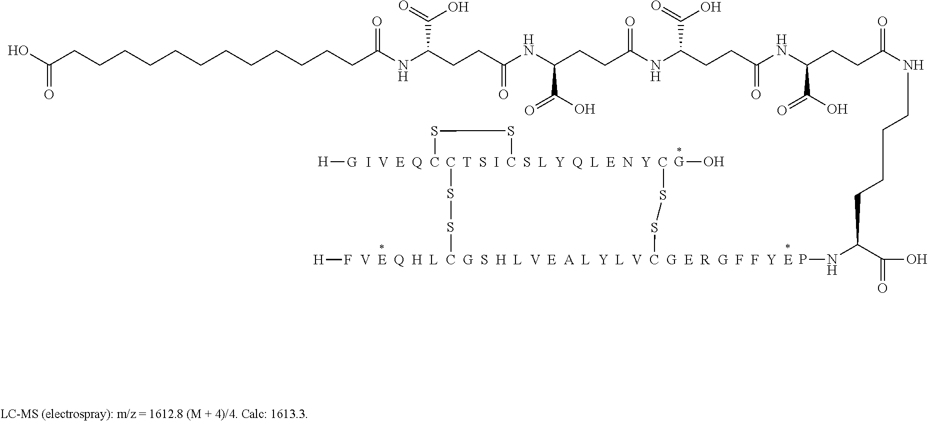

### Example 26

### General Procedure (A)

### A8H, B3E, B28D, B29K(N(eps)tetradecanedioyl-4×gGlu), desB30 Human Insulin; (SEQ ID NOS: 1 and 13)

IUPAC (OpenEye, IUPAC style) name: N{Epsilon-B29}-[(4S)-4-carboxy-4-[[(4S)-4-carboxy-4-[[(4S)-4-carboxy-4-[[(4S)-4-carboxy-4-(13-carboxytridecanoylamino) butanoyl]amino]butanoyl]amino]butanoyl]amino]butanoyl]-[HisA8,GluB3,AspB28],des-ThrB30-Insulin(human).

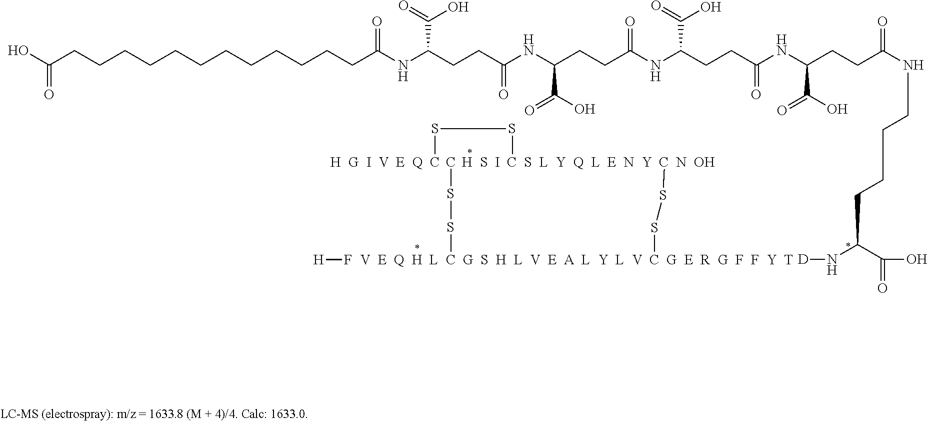

### Example 27

### General Procedure (A)

### A21A, B3E, B27E, B28E, B29K(N(eps)hexadecanedioyl-4×gGlu), desB30 Human Insulin; (SEQ ID NOS: 6 and 12)

IUPAC (OpenEye, IUPAC style) name: N{Epsilon-B29}-[(4S)-4-carboxy-4-[[(4S)-4-carboxy-4-[[(4S)-4-carboxy-4-[[(4S)-4-carboxy-4-(15-carboxypentadecanoylamino) butanoyl]amino]butanoyl]amino]butanoyl]amino]butanoyl]-[AlaA21,GluB3,GluB27,GluB28],des-ThrB30-Insulin(human).

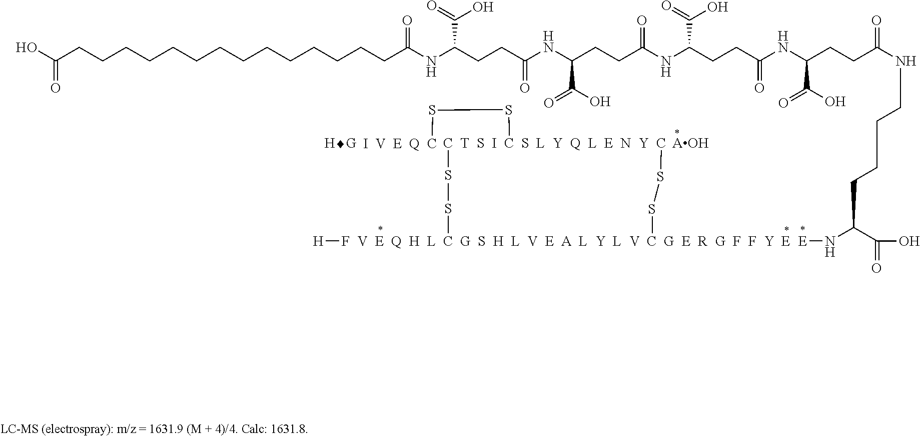

### Example 28

### General Procedure (A)

### A21G, B3E, B28D, B29K(N(eps)hexadecanedioyl-4×gGlu), desB30 Human Insulin; (SEQ ID NOS: 7 and 13)

IUPAC (OpenEye, IUPAC style) name: N{Epsilon-B29}-[(4S)-4-carboxy-4-[[(4S)-4-carboxy-4-[[(4S)-4-carboxy-4-[[(4S)-4-carboxy-4-(15-carboxypentadecanoylamino) butanoyl]amino]butanoyl]amino]butanoyl]amino]butanoyl]-[GlyA21,GluB3,AspB28],des-ThrB30-Insulin(human).

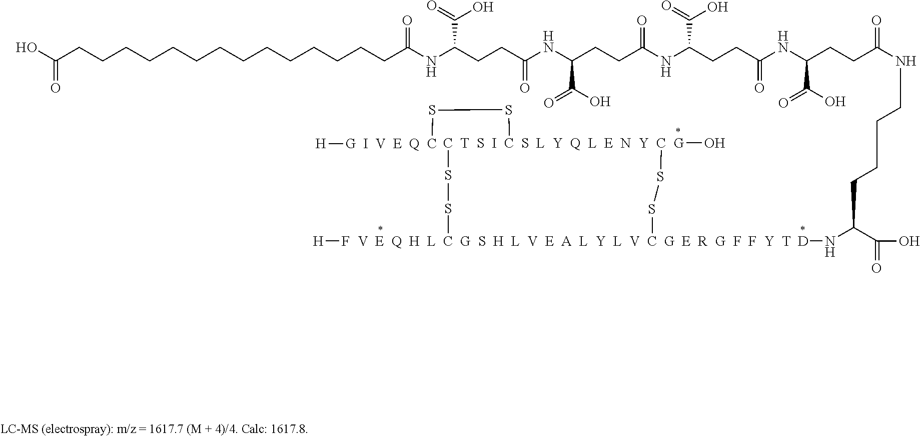

### Example 29

### General Procedure (B)

### B3E, B26E, B29K(N(eps)tetradecanedioyl-4×gGlu), desB30 Human Insulin; (SEQ ID NO:8)

IUPAC (OpenEye, IUPAC style) name: N{Epsilon-B29}-[(4S)-4-carboxy-4-[[(4S)-4-carboxy-4-[[(4S)-4-carboxy-4-[[(4S)-4-carboxy-4-(13-carboxytridecanoylamino) butanoyl]amino]butanoyl]amino]butanoyl]amino]butanoyl]-[GluB3,GluB26],des-ThrB30-Insulin(human).

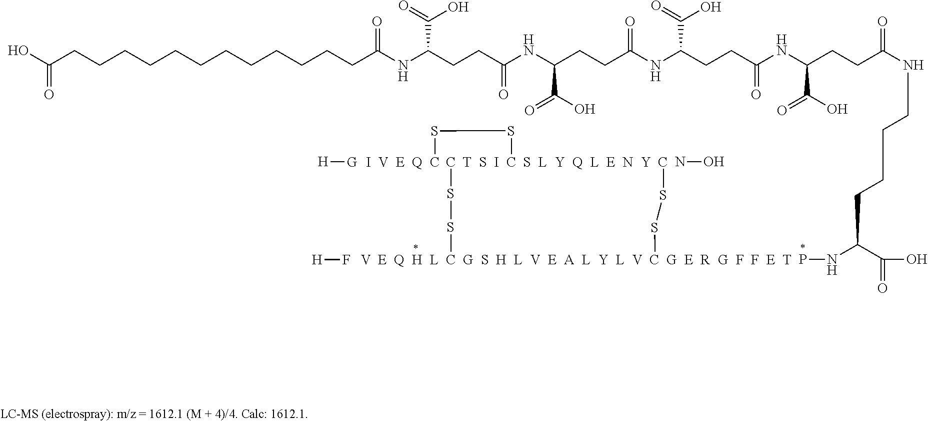

### Example 30

### General Procedure (B)

### A21A, B3E, B26E, B29K(N(eps)tetradecanedioyl-4×gGlu), desB30 Human Insulin; (SEQ ID NOS: 6 and 30)

IUPAC (OpenEye, IUPAC style) name: N{Epsilon-B29}-[(4S)-4-carboxy-4-[[(4S)-4-carboxy-4-[[(4S)-4-carboxy-4-[[(4S)-4-carboxy-4-(13-carboxytridecanoylamino) butanoyl]amino]butanoyl]amino]butanoyl]amino]butanoyl]-[AlaA21,GluB3,GluB26],des-ThrB30-Insulin(human).

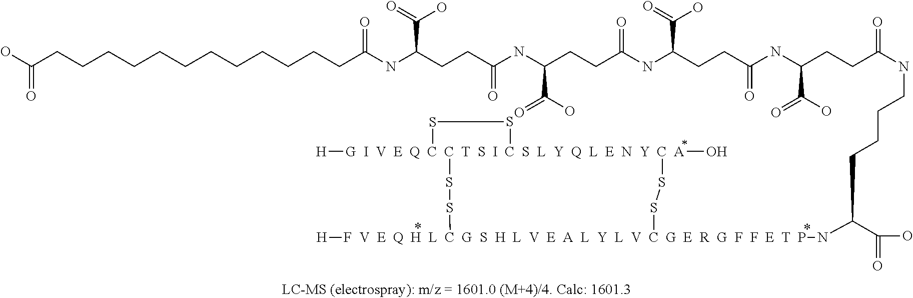

### Example 31

### May be Prepared According to General Procedure (A or B)

### B3E, B26E, B28E, B29K(N(eps)tetradecanedioyl-4×gGlu), desB30 Human Insulin; (SEQ ID NO:9)

IUPAC (OpenEye, IUPAC style) name: N{Epsilon-B29}-[(4S)-4-carboxy-4-[[(4S)-4-carboxy-4-[[(4S)-4-carboxy-4-[[(4S)-4-carboxy-4-(13-carboxytridecanoylamino) butanoyl]amino]butanoyl]amino]butanoyl]amino]butanoyl]-[GluB3,GluB26,GluB28],des-ThrB30-Insulin(human).

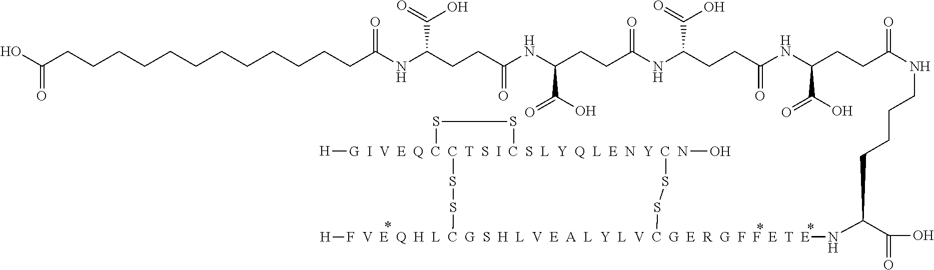

### Example 32

### May be Prepared According to General Procedure (A or B)

### A21A, B3E, B26E, B28E, B29K(N(eps)tetradecanedioyl-4×gGlu), desB30 Human Insulin; (SEQ ID NOS: 6 and 9)

IUPAC (OpenEye, IUPAC style) name: N{Epsilon-B29}-[(4S)-4-carboxy-4-[[(4S)-4-carboxy-4-[[(4S)-4-carboxy-4-[[(4S)-4-carboxy-4-(13-carboxytridecanoylamino) butanoyl]amino]butanoyl]amino]butanoyl]amino]butanoyl]-[AlaA21,GluB3,GluB26,GluB28],des-ThrB30-Insulin(human).

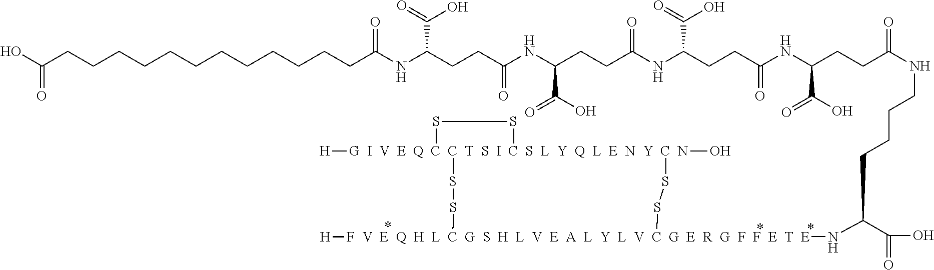

### Example 33

### General Procedure (A)

### A21G, B3E, B28D, B29K(N(eps)hexadecanedioyl-gGlu-2×OEG), desB30 Human Insulin; (SEQ ID NOS: 7 and 13)

IUPAC (OpenEye, IUPAC style) name: N{Epsilon-B29}-[2-[2-[2-[[2-[2-[2-[[(4S)-4-carboxy-4-(15-carboxypentadecanoylamino)butanoyl]amino]ethoxy]ethoxy]acetyl]-amino]ethoxy]ethoxy]acetyl]-[GlyA21,GluB3,AspB28],des-ThrB30-Insulin(human).

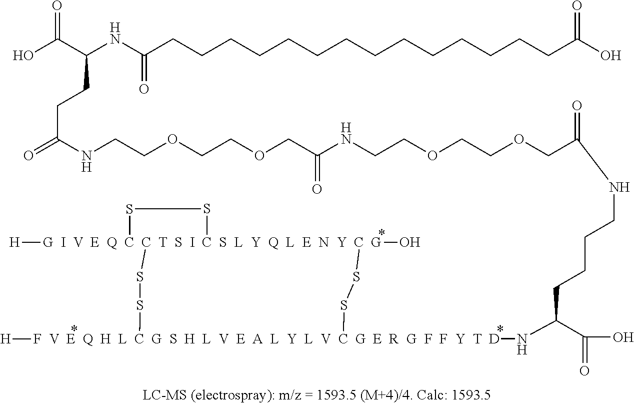

### Example 34

### General Procedure (A)

### A21G, B3E, B28E, B29K(N(eps)tetradecanedioyl-4×gGlu), desB30 Human Insulin; (SEQ ID NOS: 7 and 14)

IUPAC (OpenEye, IUPAC style) name: N{Epsilon-B29}-[(4S)-4-carboxy-4-[[(4S)-4-carboxy-4-[[(4S)-4-carboxy-4-[[(4S)-4-carboxy-4-(13-carboxytridecanoylamino) butanoyl]amino]butanoyl]amino]butanoyl]amino]butanoyl]-[GlyA21,GluB3,GluB28],des-ThrB30-Insulin(human).

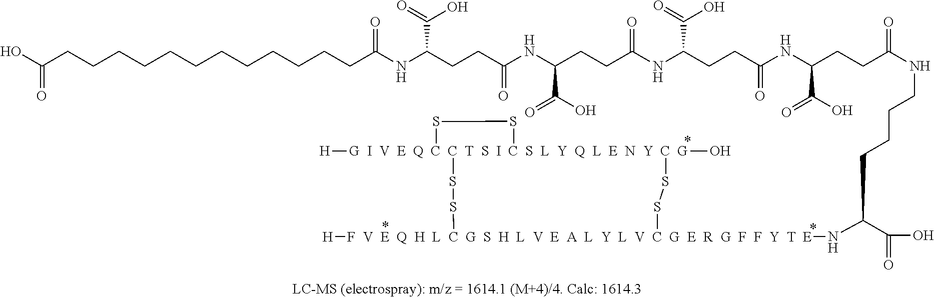

### Example 35

### General Procedure (A)

### B3E, B28E, B29K(N(eps)tetradecanedioyl-4×gGlu), desB30 Human Insulin; (SEQ ID NO:14)

IUPAC (OpenEye, IUPAC style) name: N{Epsilon-B29}-[(4S)-4-carboxy-4-[[(4S)-4-carboxy-4-[[(4S)-4-carboxy-4-[[(4S)-4-carboxy-4-(13-carboxytridecanoylamino) butanoyl]amino]butanoyl]amino]butanoyl]amino]butanoyl]-[GluB3,GluB28],des-ThrB30-Insulin(human).

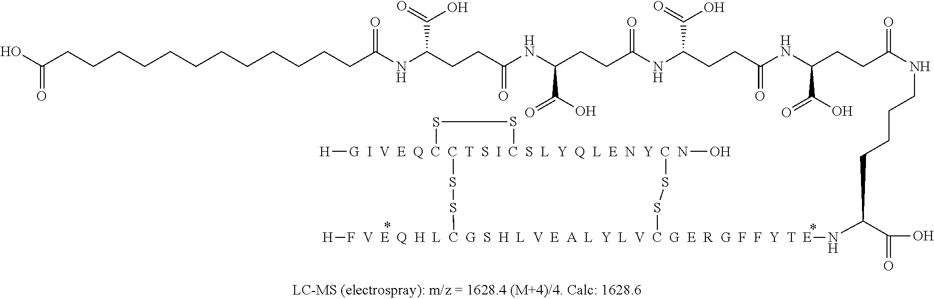

### Example 36

### May be Prepared According to General Procedure (A or B)

### B3E, B28E, B29K(N(eps)tetradecanedioyl-gGlu-2×OEG), desB30 Human Insulin; (SEQ ID NO:14)

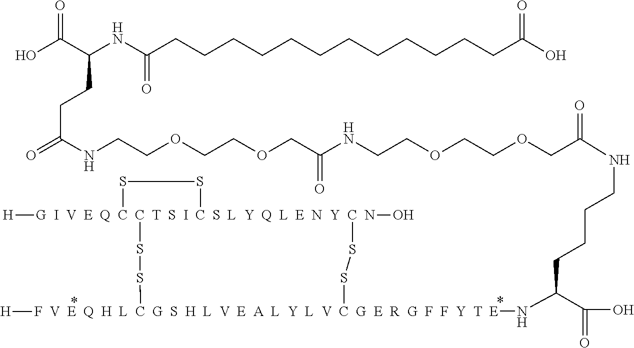

### Example 37

### May be Prepared According to General Procedure (A or B)

### A21G, B3E, B28E, B29K(N(eps)tetradecanedioyl-gGlu-2×OEG), desB30 Human Insulin; (SEQ ID NOS: 7 and 14)

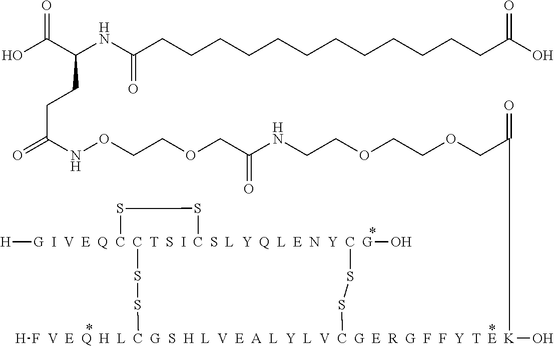

### Example 38

### General Procedure (A)

### A21A, B3E, B28E, B29K(N(eps)tetradecanedioyl-gGlu-2×OEG), desB30 Human Insulin; (SEQ ID NOS: 6 and 14)

IUPAC (OpenEye, IUPAC style) name: N{Epsilon-B29}-[2-[2-[2-[[2-[2-[2-[[(4S)-4-carboxy-4-(13-carboxytridecanoylamino)butanoyl]amino]ethoxy]ethoxy]-acetyl]amino]ethoxy]ethoxy]acetyl]-[AlaA21,GluB3,GluB28],des-ThrB30-Insulin(human).

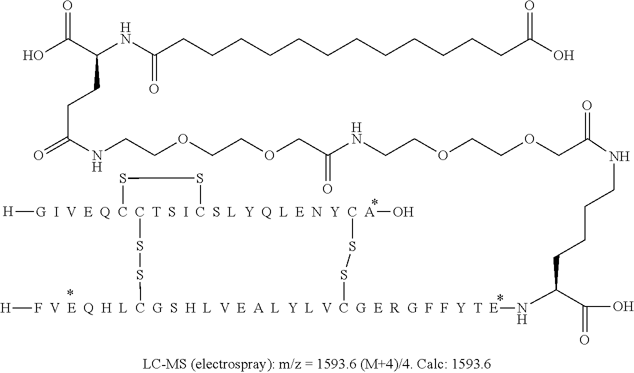

### Example 39

### May be Prepared According to General Procedure (A or B)

### B3E, B26E, B29K(N(eps)tetradecanedioyl-gGlu-2×OEG), desB30 Human Insulin; (SEQ ID NOS: 8)

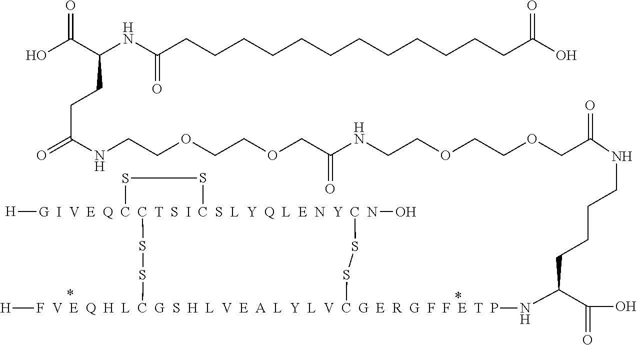

### Example 40

### May be Prepared According to General Procedure (A or B)

### A21G, B3E, B26E, B29K(N(eps)tetradecanedioyl-gGlu-2×OEG), desB30 Human Insulin; (SEQ ID NOS: 7 and 8)

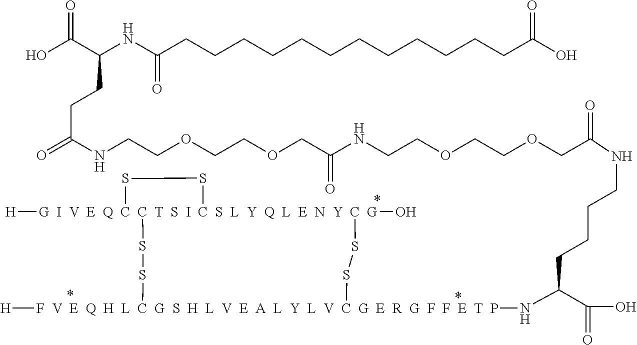

### Example 41

### May be Prepared According to General Procedure (A or B)

### A21A, B3E, B26E, B29K(N(eps)tetradecanedioyl-gGlu-2×OEG), desB30 Human Insulin; (SEQ ID NOS: 6 and 8)

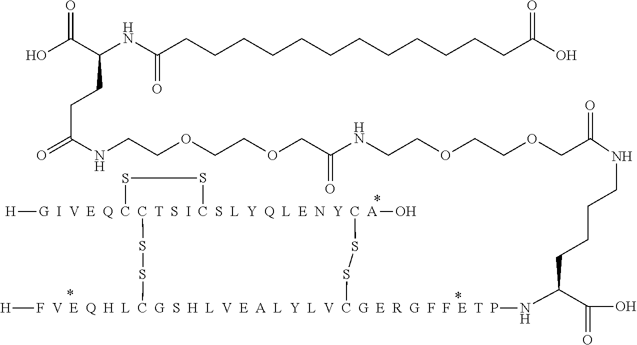

### Example 42

### May be Prepared According to General Procedure (A or B)

### A21G, B3E, B26E, B28E, B29K(N(eps)tetradecanedioyl-4×gGlu), desB30 Human Insulin; (SEQ ID NOS: 7 and 9)

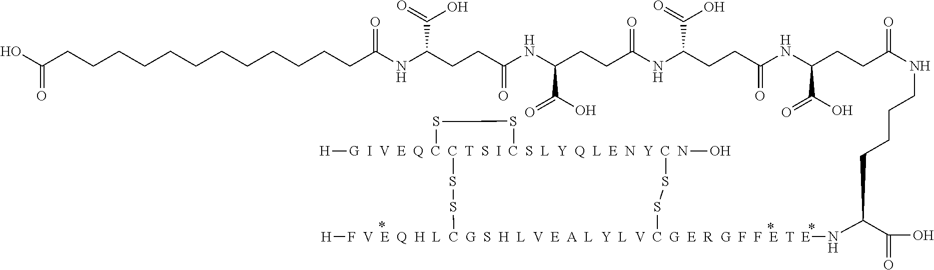

### Example 43

### May be Prepared According to General Procedure (A or B)

### B3E, B26E, B28E, B29K(N(eps)tetradecanedioyl-gGlu-2×OEG), desB30 Human Insulin; (SEQ ID NO:9)

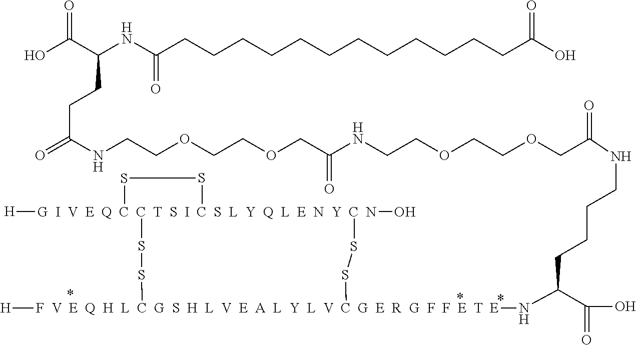

### Example 44

### May be Prepared According to General Procedure (A or B)

### A21G, B3E, B26E, B28E, B29K(N(eps)tetradecanedioyl-gGlu-2×OEG), desB30 Human Insulin; (SEQ ID NOS: 7 and 9)

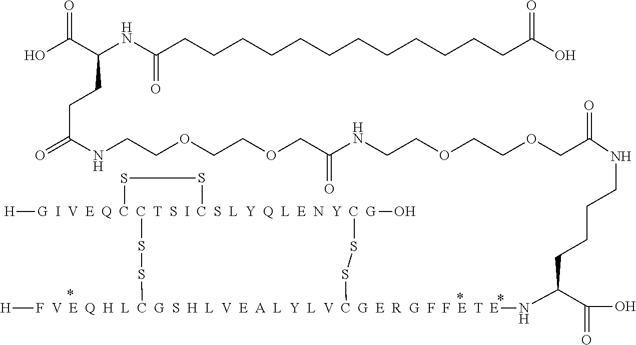

### Example 45

### May be Prepared According to General Procedure (A or B)

### A21A, B3E, B26E, B28E, B29K(N(eps)tetradecanedioyl-gGlu-2×OEG), desB30 Human Insulin; (SEQ ID NOS: 6 and 9)

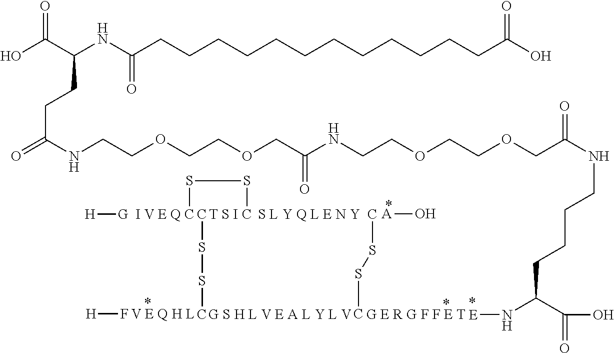

### Example 46

### May be Prepared According to General Procedure (A or B)

### A21G, B3E, B26E, B29K(N(eps)tetradecanedioyl-4×gGlu), desB30 Human Insulin; (SEQ ID NOS: 7 and 8)

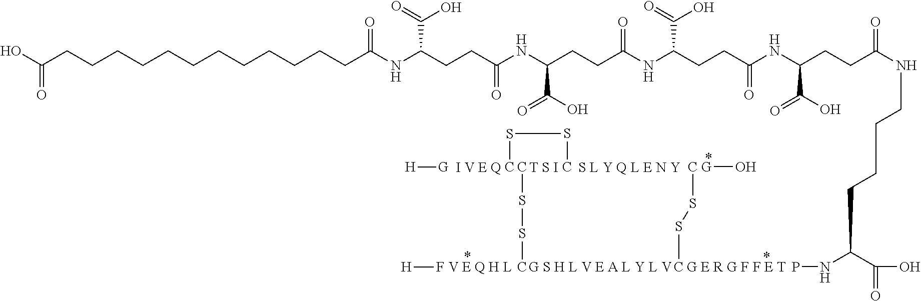

### Example 47

### General Procedure (B)

### B3Q, B26E, B29K(N(eps)tetradecanedioyl-4×gGlu), desB30 Human Insulin; (SEQ ID NO:15)

IUPAC (OpenEye, IUPAC style) name: N{Epsilon-B29}-[(4S)-4-carboxy-4-[[(4S)-4-carboxy-4-[[(4S)-4-carboxy-4-[[(4S)-4-carboxy-4-(13-carboxytridecanoylamino)butanoyl]amino]butanoyl]amino]butanoyl]amino]butanoyl]-[GlnB3,GluB26],des-ThrB30-Insulin(human)

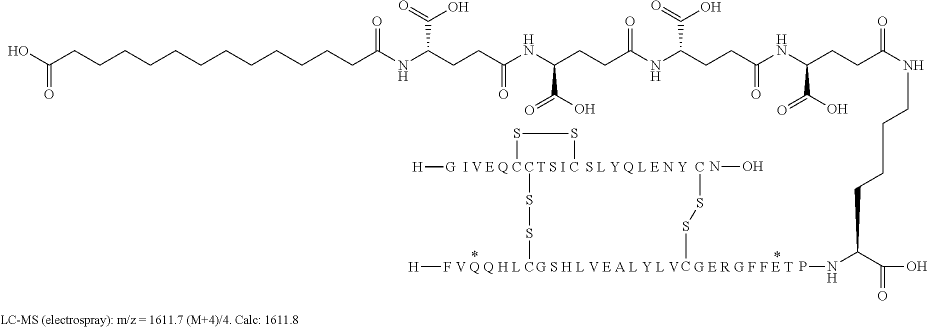

### Example 48

### General Procedure (B)

### A21A, B3Q, B28E, B29K(N(eps)tetradecanedioyl-4×gGlu), desB30 Human Insulin; (SEQ ID NOS: 6 and 17)

IUPAC (OpenEye, IUPAC style) name: N{Epsilon-B29}-[(4S)-4-carboxy-4-[[(4S)-4-carboxy-4-[[(4S)-4-carboxy-4-[[(4S)-4-carboxy-4-(13-carboxytridecanoylamino)butanoyl]amino]butanoyl]amino]butanoyl]amino]butanoyl]-[AlaA21,GlnB3,GluB28],des-ThrB30-Insulin(human)

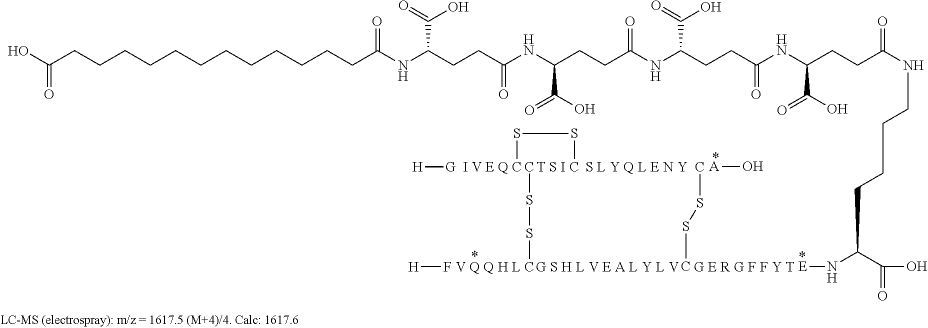

### Example 49

### B3Q, B28E, B29K(N(eps)tetradecanedioyl-4×gGlu), desB30 Human Insulin; (SEQ ID NO:17)

IUPAC (OpenEye, IUPAC style) name: N{Epsilon-B29}-[(4S)-4-carboxy-4-[[(4S)-4-carboxy-4-[[(4S)-4-carboxy-4-[[(4S)-4-carboxy-4-(13-carboxytridecanoylamino)butanoyl]amino]butanoyl]amino]butanoyl]amino]butanoyl]-[GlnB3,GluB28],des-ThrB30-Insulin(human)

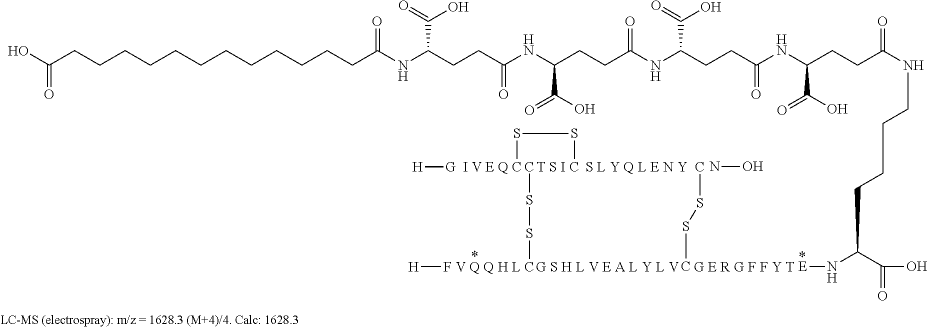

### Example 50

### General Procedure (B)

### A21A, B3Q, B26E, B29K(N(eps)tetradecanedioyl-4×gGlu), desB30 Human Insulin; (SEQ ID NOS: 6 and 15)

IUPAC (OpenEye, IUPAC style) name: N{Epsilon-B29}-[(4S)-4-carboxy-4-[[(4S)-4-carboxy-4-[[(4S)-4-carboxy-4-[[(4S)-4-carboxy-4-(13-carboxytridecanoylamino)butanoyl]amino]butanoyl]amino]butanoyl]amino]butanoyl]-[AlaA21,GlnB3,GluB26],des-ThrB30-Insulin(human)

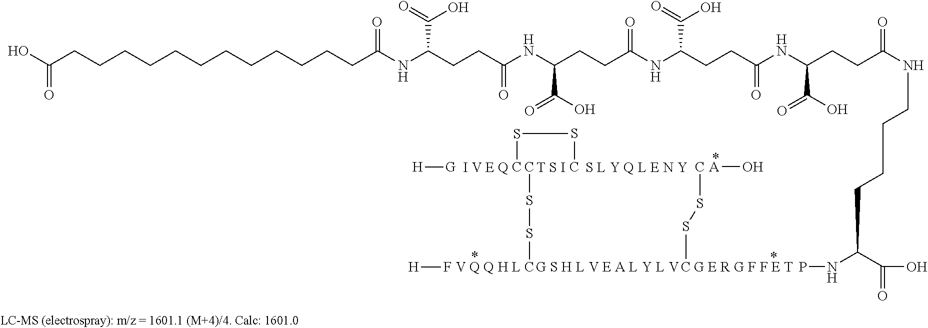

### Example 51

### General Procedure (A)

### A21A, B3E, B28E, B29K(N(eps)hexadecanedioyl-4×gGlu), desB30 Human Insulin; (SEQ ID NOS: 6 and 14)

IUPAC (OpenEye, IUPAC style) name: N{Epsilon-B29}-[(4S)-4-carboxy-4-[[(4S)-4-carboxy-4-[[(4S)-4-carboxy-4-[[(4S)-4-carboxy-4-(15-carboxypentadecanoylamino)-butanoyl]amino]butanoyl]amino]butanoyl]amino]butanoyl]-[AlaA21,GluB3,GluB28],des-ThrB30-Insulin

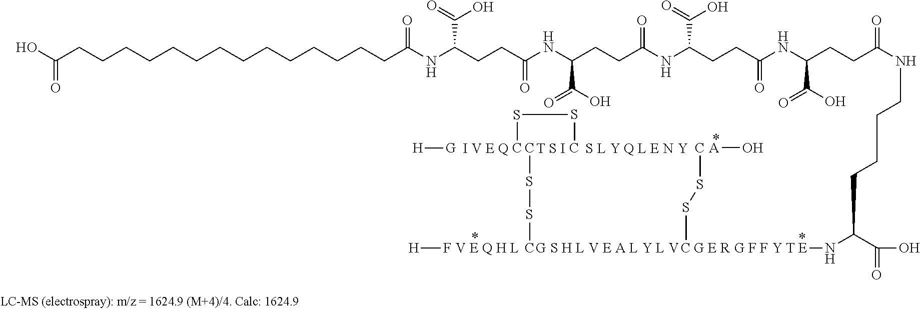

### Example 52

### General Procedure (A)

### A21A, B3E, B28E, B29K(N(eps)hexadecanedioyl-gGlu-2×OEG), desB30 Human Insulin; (SEQ ID NOS: 6 and 14)

IUPAC (OpenEye, IUPAC style) name: N{Epsilon-B29}-[2-[2-[2-[[2-[2-[2-[[(4S)-4-carboxy-4-(15-carboxypentadecanoylamino)butanoyl]amino]ethoxy]ethoxy]-acetyl]amino]ethoxy]ethoxy]acetyl]-[AlaA21,GluB3,GluB28],des-ThrB30-Insulin

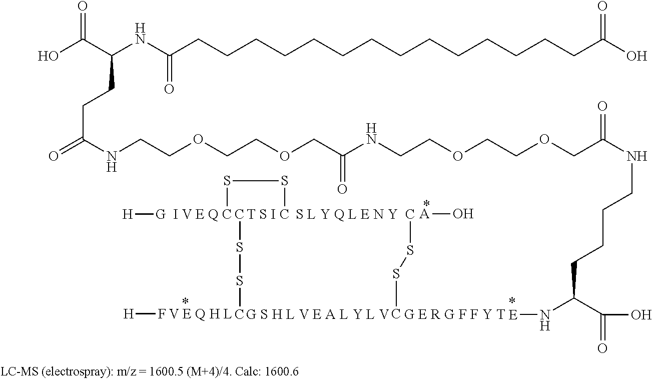

### Prior Art Analogue 1

### B29K(N(eps)hexadecanedioyl-Glu-2×OEG), desB30 Human Insulin: WO 2009 022006; Example 10

IUPAC (OpenEye, IUPAC style) name: N{Epsilon-B29}-[2-[2-[2-[[2-[2-[2-[[(4S)-4-carboxy-4-(15-carboxypentadecanoylamino)butanoyl]amino]ethoxy]ethoxy]acetyl]amino]ethoxy]ethoxy]acetyl]-des-ThrB30-Insulin(human).

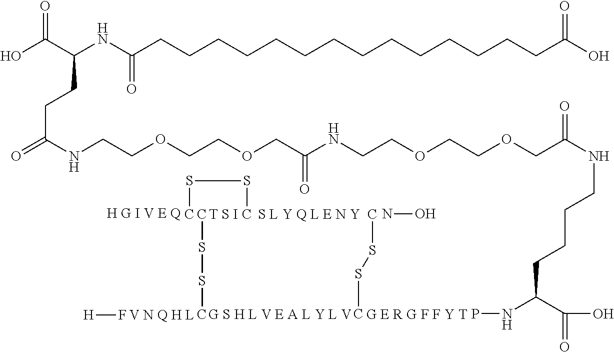

### Prior Art Analogue 2

### B28D, B29K(N(eps)tetradecanedioyl-gGlu-2×OEG), desB30 Human Insulin: Tetradecanedioic Acid Analogue of Prior Art Analogue 1 with the B28D Substitution Known from Insulin Aspart

IUPAC (OpenEye, IUPAC style) name: N{Epsilon-B29}-[2-[2-[2-[[2-[2-[2-[[(4S)-4-carboxy-4-(13-carboxytridecanoylamino)butanoyl]amino]-ethoxy]ethoxy]acetyl]amino]ethoxy]ethoxy]acetyl]-[AspB28],des-ThrB30-Insulin(human).

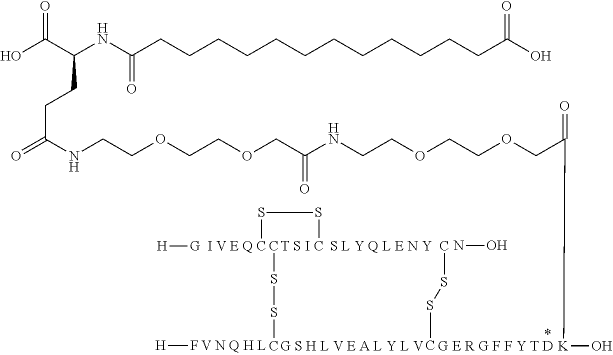

In WO 2009 022006 the substitution B28D is disclosed in a combination with an octadecanedioic acid (C18 diacid) based side chain.

### Prior Art Analogue 3

### B29K(N(eps)tetradecanedioyl), desB30 Human Insulin: WO 9731022; Example 1

IUPAC (OpenEye, IUPAC style) name: N{Epsilon-B29}-13-carboxytridecanoyl-des-ThrB30-Insulin(human).

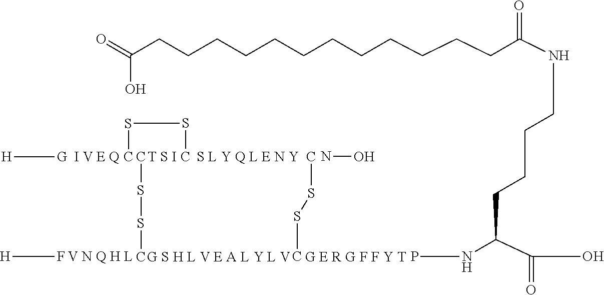

### Prior Art Analogue 4

### DesB27, B29K(N(eps)tetradecanedioyl-gGlu-2×OEG), desB30 Human Insulin: WO 2009 022006

IUPAC (OpenEye, IUPAC style) name: N{Epsilon-B29}-[2-[2-[2-[[2-[2-[2-[[(4S)-4-carboxy-4-(13-carboxytridecanoylamino)butanoyl]amino]ethoxy]ethoxy]acetyl]amino]-ethoxy]ethoxy]acetyl]-des-ThrB27,ThrB30-Insulin(human).

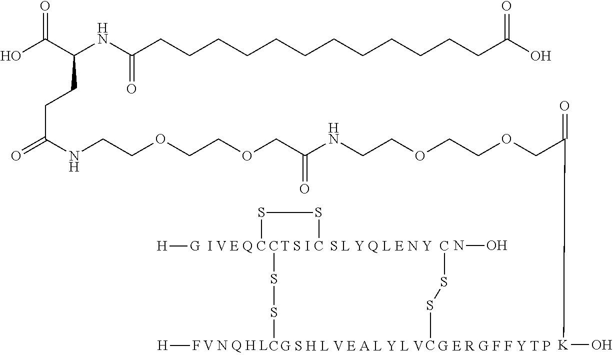

This analogue is similar to WO 2009 022006, Example 10 above (Prior Art Analogue 1), but with the following changes relative to Example 10: tetradecanedioic acid moiety replacing hexadecanedioic acid moiety of Example 10 and introduction of the desB27 mutation, not disclosed in WO 2009 022006. This is directly to assess the beneficial and unexpected effect of changing B3N (in human insulin) to B3E or B3Q.

### Prior Art Analogue 5

### B29K(N(eps)tetradecanedioyl-4×gGlu), desB30 Human Insulin: WO 2009 022006

IUPAC (OpenEye, IUPAC style) name: N{Epsilon-B29}-[(4S)-4-carboxy-4-[[(4S)-4-carboxy-4-[[(4S)-4-carboxy-4-[[(4S)-4-carboxy-4-(13-carboxytridecanoylamino) butanoyl]amino]butanoyl]amino]butanoyl]amino]butanoyl]-des-ThrB3-Insulin(human).

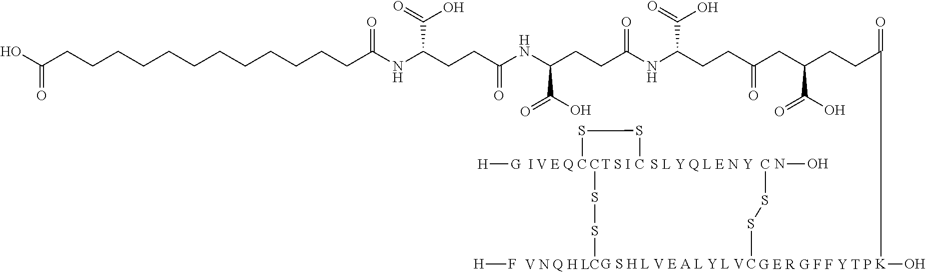

This analogue is similar to WO 2009 022006, Example 10 above (Prior Art Analogue 1), but with the following changes relative to Example 10: tetradecanedioic acid moiety replacing hexadecanedioic acid moiety of Example 10 and linker 4×gGlu replacing gGlu-2×OEG. This is directly to assess the beneficial and unexpected effect of changing B3N (in human insulin) to B3E or B3Q.

### Prior Art Analogue 6

### B29K(N(eps)tetradecandioyl-gGlu), desB30 Insulin Human Insulin: WO 2006 125765; Disclosed as a Prophetic Substance

IUPAC (OpenEye, IUPAC style) name: N{Epsilon-B29}-[(4S)-4-carboxy-4-(13-carboxytridecanoylamino)butanoyl]-des-ThrB30-Insulin(human).

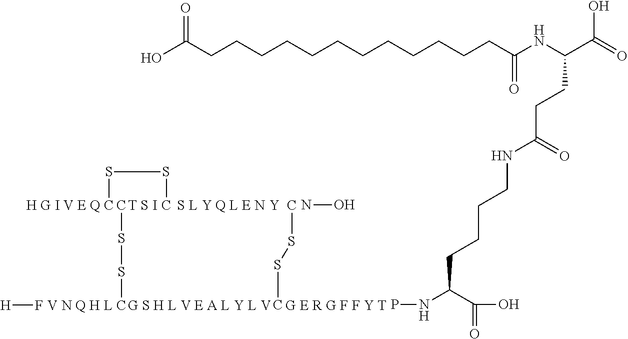

### Prior Art Analogue 7

### B29K(N(eps)hexadecanedioyl-gGlu), desB30 Insulin Human Insulin: WO 2005 012347; Examples 1 & 4; and WO 2006 125765; Examples 7, 8 and 9

IUPAC (OpenEye, IUPAC style) name: N{Epsilon-B29}-[(4S)-4-carboxy-4-(15-carboxypentadecanoylamino)butanoyl]-des-ThrB30-Insulin(human).

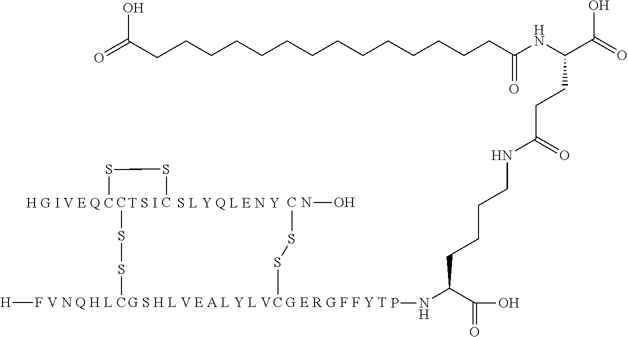

This prior art molecule is also known as insulin degludec and as Tresiba®, currently marketed in several countries for human use as a basal insulin analogue with ultra-long duration of action.

### Example 53

### Insulin Receptor Affinity of Selected Insulin Derivatives of the Invention, Measured on Solubilised Receptors

The relative binding affinity of the insulin analogues of the invention for the human insulin receptor (IR) is determined by competition binding in a scintillation proximity assay (SPA) (according to Glendorf T et al. (2008) Biochemistry 47 4743-4751).

In brief, dilution series of a human insulin standard and the insulin analogue to be tested are performed in 96-well Optiplates (Perkin-Elmer Life Sciences) followed by the addition of [¹²⁵I-A14Y]-human insulin, anti-IR mouse antibody 83-7, solubilised human IR-A (semipurified by wheat germ agglutinin chromatography from baby hamster kidney (BHK) cells overexpressing the IR-A holoreceptor), and SPA beads (Anti-Mouse polyvinyltoluene SPA Beads, GE Healthcare) in binding buffer consisting of 100 mM HEPES (pH 7.8), 100 mM NaCl, 10 mM MgSO₄, and 0.025% (v/v) Tween 20. Plates are incubated with gentle shaking for 22-24 h at 22° C., centrifuged at 2000 rpm for 2 minutes and counted on a TopCount NXT (Perkin-Elmer Life Sciences).

Data from the SPA are analysed according to the four-parameter logistic model (Vølund A (1978) Biometrics 34 357-365), and the binding affinities of the analogues calculated relative to that of the human insulin standard measured within the same plate.

A related assay is also used wherein the binding buffer contains 1.5% HSA (w/v) (Sigma A1887) in order to mimic more physiological conditions.

Insulin receptor affinities and other in vitro data of selected insulin analogues of the invention are presented in Table 1, below.

TABLE 1
Insulin receptor affinities (A and B isoforms, at 0%, 1.5%
and 0.1% HSA), IGF-1 receptor affinities at 0.1% HSA and functional
lipogenesis potencies of insulins of the invention

| | hIRA | hIRA | hIRA | hIRB | hIGF1R | Lipo- |
|---|---|---|---|---|---|---|
| | 0% | 1.5% | 0.1% | 0.1% | 0.1% | genesis |
| | HSA | HSA | HSA | HSA | HSA | 1% HSA |
| | (% rel | (% rel | (% rel | (% rel | (% rel | (% rel |
| Ex. | to HI) | to HI) | to HI) | to HI) | to HI) | to HI) |
| No. | Ex 53 | Ex 53 | Ex 54 | Ex 54 | Ex 54 | Ex 55 |
| 1 | 18.1 | 8.9 | 24.4 | 26.8 | 1.0 | 3.3 |
| 2 | 10.4 | 3.2 | 16.9 | 20.9 | 0.8 | 2.0 |
| 3 | 11.3 | 6.8 | 19.9 | 23.9 | 0.4 | 3.1 |
| 4 | 29.6 | 10.8 | 23.6 | 23.9 | 4.3 | 4.1 |
| 5 | 105.0 | 28.8 | ND | ND | ND | ND |
| 6 | 57.2 | 21.9 | 53.0 | 62.1 | 3.7 | 11.6 |
| 7 | 63.0 | 35.1 | 54.6 | 68.7 | 3.1 | 13.3 |
| 8 | 85.3 | 36.5 | 85.8 | 105.9 | 6.9 | 14.5 |
| 10 | 12.8 | 4.4 | 34.0 | 34.1 | 1.5 | 4.1 |
| 11 | 20.8 | 9.9 | 29.5 | 38.4 | 4.4 | 5.4 |
| 12 | 89.5 | 42.7 | 106.4 | 103.5 | 6.4 | 18.3 |
| 13 | 32.5 | 12.6 | 62.1 | 48.8 | 7.8 | 3.9 |
| 16 | 20.0 | 10.0 | 25.9 | 28.0 | 7.2 | 3.9 |
| 17 | 29.1 | 11.9 | 23.7 | 32.8 | 8.0 | 7.5 |
| 18 | 45.1 | 21.9 | 22.5 | 30.5 | 3.4 | 5.2 |
| 19 | 58.1 | 23.9 | 27.1 | 31.2 | 2.6 | 7.4 |
| 20 | 41.5 | 28.6 | 39.9 | 50.3 | 6.3 | 8.5 |
| 21 | 29.5 | 20.6 | 37.0 | 50.8 | 2.5 | 7.8 |
| 22 | 26.8 | 17.9 | 14.8 | 19.3 | 1.8 | 3.3 |
| 23 | 23.4 | 11.9 | 14.7 | 19.8 | 0.5 | 4.4 |
| 24 | 41.4 | 19.0 | 28.0 | 34.0 | 2.0 | 4.8 |
| 25 | 8.8 | 5.0 | 14.3 | 15.5 | 0.8 | 3.9 |
| 27 | ND | 1.8 | 7.1 | 11.3 | 2.0 | 0.68 |
| 28 | 1.3 | ND | 6.9 | 9.4 | 1.1 | 0.69 |
| 26 | 171 | 93.5 | 69.2 | 88.0 | 3.4 | 24.7 |
| 29 | ND | 41.7 | 27.3 | 30.3 | 0.80 | 6.2 |
| 30 | ND | 16.7 | 21.0 | 21.0 | 0.83 | 3.7 |
| 31 | ND | ND | ND | ND | ND | ND |
| 32 | ND | ND | ND | ND | ND | ND |
| 35 | ND | 22.6 | 19.4 | 35.7 | 0.1 | ND |
| 33 | 13.4 | 0.52 | 8.71 | 7.32 | 1.60 | 0.39 |
| 34 | ND | 11.3 | 19.7 | 12.4 | 2.1 | 5.5 |
| 38 | 52.8 | 18.9 | 36.1 | 39.1 | ND | ND |
| 47 | 58.0 | 31.3 | 30.8 | 32.5 | 0.99 | 7.9 |
| 48 | 54.9 | 30.4 | 39.8 | 50.4 | 2.64 | 7.5 |
| 49 | 45.9 | 21.4 | 27.4 | 34.3 | 2.19 | 6.0 |
| 50 | 41.1 | 13.3 | 29.7 | 33.6 | 0.65 | 4.4 |
| 51 | 43.1 | 2.64 | 10.0 | 11.9 | ND | ND |
| 52 | 40.3 | 2.27 | 9.3 | 9.0 | ND | ND |

ND: Not determined

### Example 54

### Insulin and Insulin-Like Growth Factor-1 Receptor Affinities of Selected Insulin Derivatives of the Invention, Measured on Membrane Associated Receptors

Membrane-associated human IR and IGF-1R are purified from BHKcells stably transfected with the pZem219B vector containing either the human IR-A, IR-B or IGF-IR insert. BHK cells are harvested and homogenized in ice-cold buffer (25 mM HEPES pH 7.4, 25 mM CaCl₂ and 1 mM MgCl₂, 250 mg/L bacitracin, 0.1 mM Pefablock). The homogenates are layered on a 41% (w/v) sucrose cushion and centrifuged for 75 minutes at 95000 g at 4° C. The plasma membranes are collected, diluted 1:5 with buffer (as above) and centrifuged again for 45 minutes at 40000 g at 4° C. The pellets are re-suspended in a minimal volume of buffer and drawn through a needle (size 23) three times before storage at −80° C. until usage.

The relative binding affinity for either of the membrane-associated human IR-A, IR-B or IGF-1R is determined by competition binding in a SPA setup. IR assays are performed in duplicate in 96-well OptiPlates (Perkin-Elmer Life Sciences). Membrane protein is incubated with gentle agitation for 150 minutes at 25° C. with 50 μM [¹²⁵I-A14Y]-human insulin in a total volume of 200 μL assay buffer (50 mM HEPES, 150 mM NaCl, 5 mM MgSO₄, 0.01% Triton X-100, 0.1% (w/v) HSA (Sigma A1887), Complete EDTA-free protease inhibitors), 50 μg of wheat germ agglutinate (WGA)-coated PVT microspheres (GE Healthcare) and increasing concentrations of ligand. Assays are terminated by centrifugation of the plate at 2000 rpm for 2 minutes and bound radioactivity quantified by counting on a TopCount NXT (Perkin-Elmer Life Sciences).

IGF-1R assays are conducted essentially as for the IR binding assays except that membrane-associated IGF-1R and 50 pM [¹²⁵I-Tyr31]-human IGF-1 were employed. Data from the SPA are analysed according to the four-parameter logistic model (Vølund A (1978) Biometrics 34 357-365), and the binding affinities of the analogues to be tested are calculated relative to that of the human insulin standard measured within the same plate.

IR (A isoform), IR (B isoform), and IGF-1R binding data of selected insulin analogues of the invention are given in the table above.

### Example 55

### Lipogenesis in Rat Adipocytes

As a measure of in vitro potency of the insulins of the invention, lipogenesis can be used.

Primary rat adipocytes are isolated from the epididymale fat pads and incubated with 3H-glucose in buffer containing e.g. 0.1% fat free HSA and either standard (human insulin, HI) or insulin of the invention. The labelled glucose is converted into extractable lipids in a dose dependent way, resulting in full dose response curves. The result is expressed as relative potency (%) with 95% confidence limits of insulin of the invention compared to standard (HI).

Data are given in the Table 1, above.

### Example 56

### Self-Association Measured by Small Angle X-Ray Scattering (SAXS)

SAXS data was used to estimate the self-association state of the insulin analogues to be tested after subcutaneous injection. SAXS data were collected from Zn-free formulations containing 0.6 mM of insulin analogue to be tested and 140 mM NaCl at pH 7.4. For each analogue, the relative amounts of monomer, dimer and larger species was estimated using the fact that a SAXS scattering profile has an intensity contribution from all individual components in a multicomponent mixture. By using intensities (form factors) from each component it is possible to estimate the volume fraction contribution of each component in the mixture. A system of linear equations using the algorithm of nonnegative or unconstrained least-squares is used to minimize the discrepancy between the experimental and calculated scattering curves. Form factors are calculated from crystal structures of a monomer, dimer, hexamer etc. The volume fractions are expressed in percentages (%).

Results obtained from derivatives of the invention and of derivatives of the prior art are shown in Table 2, below.

TABLE 2
SAXS data of derivatives of the invention,
and of acylated analogues of the prior art

| Ex. | SAXS* | | SAXS* | |
|---|---|---|---|---|
| No.^{a} | M + D | >D | M | D |
| 1 | 99 | 0 | 85 | 14 |
| 2 | 99 | 0 | 94 | 5 |
| 3 | 98 | 2 | 98 | 0 |
| 4 | 99 | 1 | 93 | 6 |
| 13 | 94 | 6 | 94 | 0 |
| 16 | 97 | 3 | 89 | 8 |
| 17 | 99 | 1 | 85 | 14 |
| 20 | 97 | 3 | 87 | 10 |
| 21 | 99 | 1 | 83 | 16 |
| 24 | 93 | 7 | 88 | 5 |
| PA 2 | 98 | 2 | 81 | 17 |
| PA 3 | 65 | 35 | 52 | 13 |
| PA 4 | 98 | 2 | 87 | 11 |
| PA 5 | 87 | 13 | 66 | 21 |
| PA 6 | 80 | 20 | 59 | 21 |
| PA 7 | 74 | 26 | 22 | 52 |

^{a}PA refers to Prior Art compound
*M: Percentage of monomeric species in formulation; D: Percentage of dimeric species in formulation; >D: Percentage of species larger than dimeric in formulation; M + D: Percentage of sum of monomeric and dimeric species in formulation.

It can be concluded from these studies that the derivatives of the invention, at conditions mimicking conditions in the subcutaneous tissue after injection, are much more prone to dissociate into monomers and will thus be absorbed much more quickly after subcutaneous injection than similar analogues of the prior art. The combined monomeric and dimeric content in analogues with B3E ranges from 97-99% for the analogues of the invention with very little content of species larger than dimers (3% at most). Corresponding data for analogues with B3Q Indicates slightly less content of monomer and dimer, 93%.

The majority of the analogues of the prior art are composed of much larger species than the analogues of the invention, with only two exceptions (Prior Art Analogue 2 and 4). These two analogues are not stable in formulations without zinc and are associated with prolonged PK profiles that are not suited for prandial dosing if formulated with zinc.

### Example 57

### Preparation of Pharmaceutical Preparations

The pharmaceutical preparations of the present invention may be formulated as an aqueous solution. The aqueous solution is made isotonic, for example, with sodium chloride and/or glycerol. Furthermore, the aqueous medium may contain buffers and preservatives. The pH value of the preparation is adjusted to the desired value and may be between about 3 to about 8.5, between about 3 and about 5, or about 6.5, or about 7.4, or about 7.5, depending on the isoelectric point, pI, of the insulin analogue in question.

### Preparation of Zinc-Free Insulin Formulations

Zinc-free insulin analogues were dissolved in aqueous solution, which in the final formulation contained 0.6 mM insulin analogue, 16 mM m-cresol, 16 mM phenol, and appropriate amounts of nicotinamide and glycerol, and the pH was adjusted to 7.3-7.5 (measured at room temperature) using 1 N hydrochloric acid/1 N NaOH. Water was added to the final volume and the solution was sterile-filtered through a 0.2 μm filter. The formulation was filled into 2 ml vials and sealed using crimp caps.

TABLE 3
Exemplary compositions of insulin preparations

| | Insulin | | m- | Glycerol | |
|---|---|---|---|---|---|
| Formu- | derivative | Phenol | cresol | (% | |
| lation | (mM) | (mM) | (mM) | w/v) | pH |
| A | 0.6 | 16 | 16 | 2.0 | 7.4 |
| B | 0.6 | 16 | 16 | 1.6 | 7.4 |
| C | 0.6 | 16 | 16 | 1.7 | 7.4 |

### Example 58

### ThT Fibrillation Assay for the Assessment of Physical Stability of Protein Formulations

Low physical stability of a peptide may lead to amyloid fibril formation, which is observed as well-ordered, thread-like macromolecular structures in the sample eventually resulting in gel formation. Thioflavin T (ThT) has a distinct fluorescence signature when binding to fibrils [Naiki et al. (1989) Anal. Biochem. 177 244-249; LeVine (1999) Methods. Enzymol. 309 274-284].

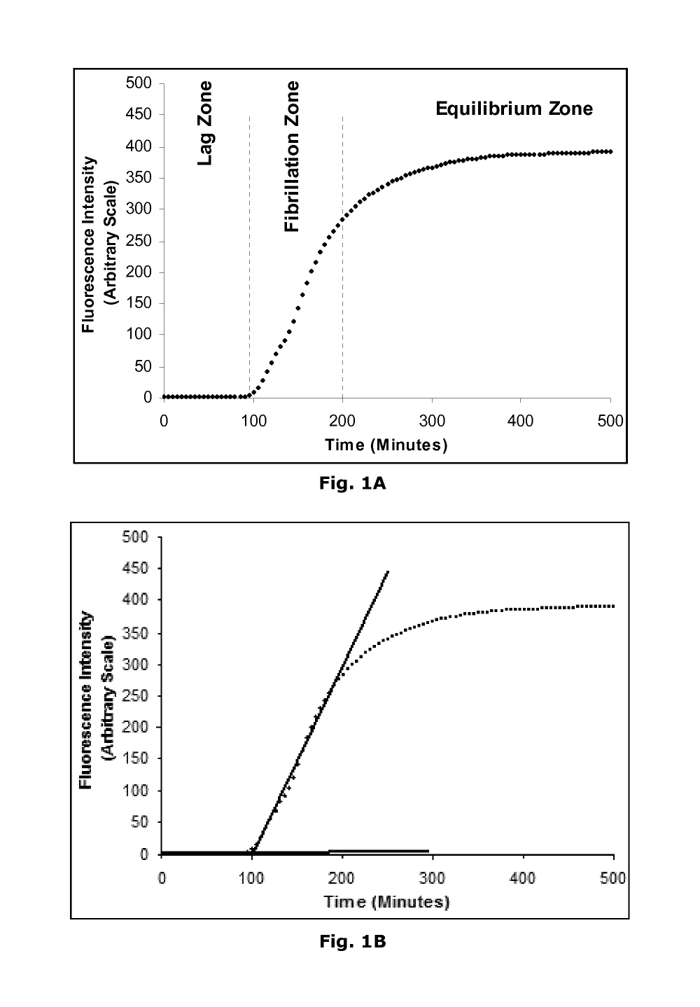

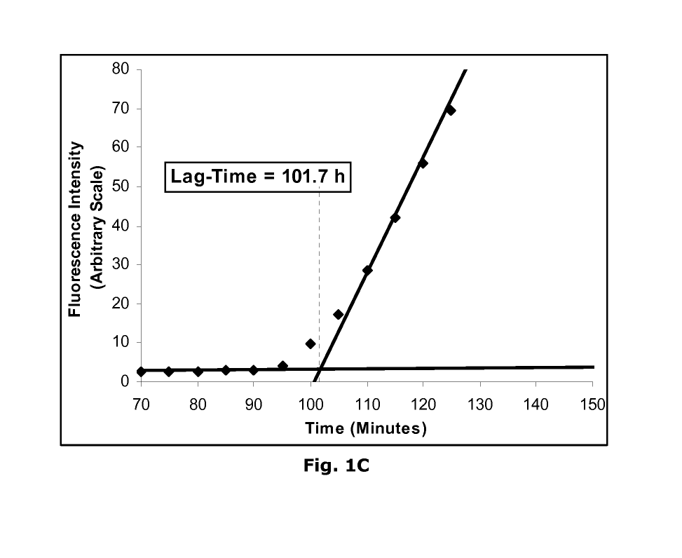

Formation of a partially folded intermediate of the peptide is suggested as a general initiating mechanism for fibrillation. Few of those intermediates nucleate to form a template onto which further intermediates may assemble and the fibrillation proceeds. The lag-time corresponds to the interval in which the critical mass of nucleus is built up and the apparent rate constant is the rate with which the fibril itself is formed (FIG. 1A).

### Sample Preparation

Samples were prepared freshly before each assay. Samples of each composition was mixed with an aqueous ThT-solution (0.1 mM ThT) in a volumetric ratio of 990:10 and transferred to a 96 well microtiter plate (Packard Opti-Plate™-96, white polystyrene). Usually, four or eight replica of each sample (corresponding to one test condition) were placed in one column of wells. The plate was sealed with Scotch 15 Pad (Qiagen).

### Incubation and Fluorescence Measurement

Incubation at given temperature, shaking and measurement of the ThT fluorescence emission were done in a Fluoroskan Ascent FL fluorescence platereader or Varioskan plate reader (Thermo Labsystems). The temperature was adjusted to 37° C. The orbital shaking was adjusted to 960 rpm with an amplitude of 1 mm in all the presented data. Fluorescence measurement was done using excitation through a 444 nm filter and measurement of emission through a 485 nm filter. Each run was initiated by incubating the plate at the assay temperature for 10 minutes. The plate was measured every 20 minutes for up to 45 hours. Between each measurement, the plate was shaken and heated as described.

### Data Handling

Fluorescence vs. time plots were generated in Microsoft Excel and the lag time was estimated as the intercept between linear approximation of the Lag Zone and Fibrillation Zone as illustrated in FIGS. 1A, 1B and 1C. An increase in lag-time corresponds to an increased physical stability. The data points are typically a mean of four or eight samples.

Results obtained for the acylated analogues if the invention, and of similar acylated analogues of the prior art are shown in Table 4, below.

TABLE 4
Physical stability measured as ThT
lad time of zinc-free preparations

| Ex. | Formu- | Lag time (h) |
|---|---|---|
| No.^{a} | lation | in ThT assay |
| 2 | C | 5 |
| 3 | C | 6 |
| 1 | C | 7 |
| 4 | B | 41 |
| 16 | C | 27 |
| 17 | B | 13 |
| 20 | B | 11 |
| 21 | B | 19 |
| PA 3 | A | 1 |
| PA 7 | A | 1 |
| PA 6 | A | 3 |
| PA 1 | A | 1 |
| PA 4 | A | 1 |
| PA 5 | A | 2 |

^{a}PA refers to Prior Art compound

It is concluded that the B29K acylated insulin analogues of the invention display better or similar stability towards fibrillation (i.e. have increased physical stability) in zinc-free formulation than similar analogues of the prior art. This is very surprising since SAXS data indicate that the insulin analogues of the invention are smaller in size (i.e. composed of monomers and dimers) which the skilled person would expect would lead to decreased physical stability.

### Example 59

### Analysis of Insulin Chemical Stability

### Size Exclusion Chromatography

Formulations used: See Example 51

Quantitative determination of high molecular weight protein (HMWP) and monomer insulin analogue was performed on Waters Acquity BEH200 SEC column (150×2.4 mm, part no. 186005225) with an eluent containing 55% (v/v) acetonitrile, 0.05% TFA at a flow rate of 0.2 ml/min and a column temperature of 40° C. Detection was performed with a tuneable absorbance detector (Waters Acquity TUV) at 215 nm. Injection volume was 1.5 μl of both the 600 μM insulin analogue formulations and a 600 μM human insulin standard. Each analogue preparation was incubated at 5, 25 and 37° C. in 2 ml vials. At defined times HMWP and content of the preparations were measured. The results are shown in Table 5, below.

TABLE 5
HMWP content by storage at 37° C.
Delta-values from start are given in parentheses

| Ex. | | 2 weeks | 4 weeks | 5 weeks | 5 weeks |
|---|---|---|---|---|---|
| No.^{a} | Start | 37° C. | 37° C. | 30° C. | 37° C. |
| 2 | 0.1% | 0.1% | ND | 0.1% | 0.2% |
| | | (+0%) | | (+0%) | (+0.1%) |
| 4 | 2.0% | 2.1% | ND | ND | ND |
| | | (+0.1%) | | | |
| 13 | 0.8 | 0.8% | ND | 0.8% | ND |
| | | (+0%) | | (+0%) | |
| 16 | 0.2% | 0.2% | ND | 0.2% | 0.2% |
| | | (+0%) | | (+0%) | (+0%) |
| 17 | 0.4% | 0.2% | 0.3% | ND | ND |
| | | (+0%) | (+0%) | | |
| 20 | 1.8% | 1.6% | 1.6% | ND | ND |
| | | (+0%) | (+0%) | | |
| 21 | 0.8% | 0.7% | 0.7% | ND | ND |
| | | (+0%) | (+0%) | | |
| 24 | 0.9% | 1.2% | ND | 1.1% | ND |
| | | (+0.3%) | | (+0.2%) | |
| PA 2 | 0.4% | 0.8% | ND | 0.7% | 1.1% |
| | | (+0.4%) | | (+0.3%) | (+0.7%) |
| PA 7 | 0.1% | 1.0% | ND | ND | 2.3% |
| | | (+0.9%) | | | (+2.2%) |
| PA 6 | 0.4% | 1.2% | ND | ND | 2.3% |
| | | (+0.8%) | | | (+1.9%) |
| PA 1 | 2.2% | 3.7% | ND | ND | 5.7% |
| | | (+1.5%) | | | (+3.5%) |
| PA 5 | 0.4% | 0.5% | ND | ND | 0.8% |
| | | (+0.1%) | | | (+0.4%) |
| PA 4 | 1.1% | 1.3% | ND | ND | 1.9% |
| | | (+0.2%) | | | (+0.8%) |

ND: Not determined
^{a}PA refers to Prior Art compound

It is concluded that formation of high molecular weight proteins (HMWP) by storage in zinc-free formulation at 37° C. is very, very low, and less than or similar to similar insulin derivatives of the prior art.

### Reverse Phase Chromatography (UPLC)

Determination of the insulin related impurities were performed on a UPLC system using a CSH Phenyl-Hexyl column, (2.1×150 mm, 1.7 μm) (Waters part no. 186005408), with a flow rate of 0.3 ml/min at 30° C. and with UV detection at 215 nm. Elution was performed with a mobile phase consisting of the following: A: 10% (v/v) acetonitrile, 100 mM di-ammonium hydrogen phosphate, pH 3.6, and B: 80% (v/v) acetonitrile. Gradient: 0-3 min linear change from 26% B to 28.5% B, 3-34 min linear change to 37% B, 34-36 minutes linear change to 80% B for column wash, before returning to initial conditions at 39 min 26% B. The amount of impurities was determined as absorbance area measured in percent of total absorbance area determined after elution of the preservatives. Each analogue preparation was incubated at 5, 25 and 37° C. in 2 ml vials. At defined times the insulin related impurities of the preparations was measured.

The results are shown in Table 6, below.

TABLE 6
Purity by storage at 37° C.
Delta-values from start are given in parentheses

| Ex. | | 2 weeks | 4 weeks | 5 weeks |
|---|---|---|---|---|
| No.^{a} | Start | 37° C. | 37° C. | 37° C. |
| 2 | 96.4% | 94.5% | ND | 90.7% |
| | | (−1.9%) | | (−5.7%) |
| 4 | 95.5% | 93.5% | ND | ND |
| | | (−2.0%) | | |
| 16 | 97.5% | 95.0% | ND | 91.5% |
| | | (−2.5%) | | (−6.0%) |
| 17 | 94.7% | 92.7% | 90.5% | ND |
| | | (−2.0%) | (−4.2%) | |
| 20 | 86.8% | 84.6% | 82.9% | ND |
| | | (−2.2%) | (−3.9%) | |
| 21 | 94.0% | 92.9% | 91.7% | ND |
| | | (−1.1%) | (−2.3%) | |
| PA 2 | 91.5% | 83.9% | | 73% |
| | | (−7.6%) | | (−18.5) |
| PA 7 | 97.8% | 89.2% | ND | 79.4% |
| | | (−8.6%) | | (−18.4%) |
| PA 6 | 95.9% | 87.3% | ND | 77.1% |
| | | (−8.6%) | | (−18.8%) |
| PA 1 | 94.5% | 86.0% | ND | 75.3% |
| | | (−8.5%) | | (−19.2%) |
| PA 5 | 93.5% | 86.5% | ND | 76.6% |
| | | (−7.0%) | | (−16.9%) |
| PA 4 | 92.4% | 83.3% | ND | 72.1% |
| | | (−9.1%) | | (−20.3%) |

^{a}PA refers to Prior Art compound
ND: Not determined

It is concluded that the insulin derivatives of the invention are far more stable in formulation without zinc than a similar B29K acylated analogue of the prior art. The analogues of the prior art are so unstable that the purity loss of Prior Art Analogue 2 after 2 weeks storage at 37° C. (loss of 7.6% purity) is larger than the purity loss of all the analogues of the invention after 5 weeks storage at 37° C. Similarly, after 5 weeks of storage at 37° C., the purity loss of prior art analogues is around 20%, which makes these analogues inappropriate for formulation without zinc. The insulin analogues of the invention (represented by the compounds of Examples 2, 4, 17, 20 and 21) have less than 2.5% points purity loss, respectively, after 2 weeks of storage at 37° C. Further, for the compounds of Examples 2, 17, 20 and 21 the purity loss after storage at 37° C. for 5 weeks is −5.7%, −4.2%, −3.9%, and −2.3% respectively, far less purity loss than observed with Prior Art Analogue 2 (−7.6% after 2 weeks and −18.9% after 5 weeks at 37° C., respectively. It is thus concluded that the insulin derivatives of the invention are stable in zinc-free formulation contrary to similar analogues of the prior art.

The acylated analogues of the prior art all need presence of zinc in the formulation in order to be stable enough for clinical use.

### Example 60

### Subcutaneous PK/PD Profiles in LYD Pigs

The insulin derivatives of the invention may be tested by subcutaneous administration to pigs, e.g. comparing with insulin aspart (NovoRapid) in the commercial formulation or comparing with similar B29K acylated insulin analogues of the prior art according to this protocol. The derivatives may be tested for pharmacokinetic and/or pharmacodynamic parameters.

### General Methods Used

### Ultrasound Examination and Marking of Injection Area

During anaesthesia for placement of permanent intravenous catheters, the pigs are examined by ultrasound with and Esaote ultrasound scanner model “MyLabFive” and a linear probe type “LA435 6-18 MHz”. Mid neck between ear and scapula, on the right or left side (opposite the catheter), an area of 2×2 cm with no underlying muscle (suitable for subcutaneous injection) is identified and marked by tattoo.

### Feeding Schedule

The pigs are fasted (no breakfast) prior to the experiment.

The pigs are in their normal pens during the entire experiment and they are not anaesthetized. The pigs are fasted until the 12-hour blood sample has been collected, but with free access to water. After the 12-hour blood sample the pigs are fed food and apples.

### Dosing

The Penfill is mounted in a NovoPen®4. A new needle is used for each pig. A needle stopper is used to secure max sc penetration to 5 mm below the epidermis. Dose volume (IU volume) is calculated and noted for each pig.

Dose volume (U)=((Weight×dose nmol/kg)/conc nmol/mL)×100 U/mL

The pig is dosed in the subcutis laterally on the right or left side (opposite the catheter) of the neck and the needle is kept in the subcutis for a minimum of 10 seconds after injection to secure deposition of compound.

### Treatment of Hypoglycaemia

After subcutaneous dosing, glucose solution should be ready for i.v. injection to prevent hypoglycaemia, i.e. 4-5 syringes (20 mL) are filled with sterile 20% glucose, ready for use. Diagnosis of hypoglycemia is based on clinical symptoms and blood glucose measurements on a glucometer (Glucocard X-meter).

Treatment consists of slow i.v. injection of 50-100 ml 20% glucose (10-20 g glucose). The glucose is given in fractions over 5-10 minutes until effect.

### Blood Sampling

The patency of the jugular catheters is checked prior to the experiment with sterile 0.9% NaCl without addition of 10 IU/mL heparin.

Before and after the dosing, blood samples will be taken in the stable from a central venous catheter at the following time points:

Predose (−10, 0), 3, 6, 9, 12, 15, 20, 30, 45, 60, 90, 120, 150, 180, 240, 300, 360, 420, 480, 540, 600 and 720 minutes

Samples are taken with a 3-way stop-cock. 4-5 ml of waste blood is withdrawn and discarded before taking the sample.

Blood samples of 0.8 ml are collected into tubes coated with EDTA for glucose and insulin analysis.

After each blood sample the catheter is flushed with 5 ml of sterile 0.9% NaCl without addition of 10 IU/mL heparin.

The tube is tilted gently a minimum of 10 times to ensure sufficient mixing of blood and anticoagulant (EDTA) and after one minute it is placed on wet ice. The tubes are spun for 10 min at 3000 rpm and 4° C. within 1 hour after sampling. The samples are stored on wet ice until pipetting.

Aseptic technique is demanded to avoid bacterial growth in the catheter with increased risk of clotting.

Closure of the Catheters after the Experiment

If blood sampling has not been performed using an aseptic technique, a single intravenous treatment with 1 ml per 10 kg Pentrexyl® (1 g of ampicillin dissolved in 10 ml 0.9% NaCl) can be administered slowly i.v. via the catheter that has been used for blood sampling. Following this treatment, the catheter is flushed with 10 ml 0.9% NaCl.

Catheters are flushed with 5 ml of sterile 0.9% NaCl added heparin (10 IU/mL). The catheters are closed with a new luer-lock with latex injection membrane and 1.0 ml of TauroLockHep500 is injected through the membrane as a lock for the catheter.

### Analysis of Blood Samples

Plasma glucose: 10 ul of plasma is pipetted into 500 ul of buffer solution for measurements of glucose concentration in plasma in the BIOSEN autoanalyser.

Plasma insulin: 1×50 μl of plasma are pipetted into 0.65 ml Micronic® tubes (ELISA/LOCI/SPA setup) for analysis, using either ELISA or LC-MS.

Plasma is stored frozen at −20° C.

### Example 61

### Subcutaneous PK/PD Profile of the Insulin of Example 16 in LYD Pigs

Following the general procedure above, the following PK and PD profiles were obtained for the insulin derivative of Example 16.

### Formulations Used

The compound of Example 16, pH=7.38; 622.3 μM; 7 mM phosphate; 1.6% (w/vol) glycerol; 16 mM phenol; 16 mM m-cresol; 10 mM sodium chloride (0 Zn/hexamer); 1 nmol/kg.

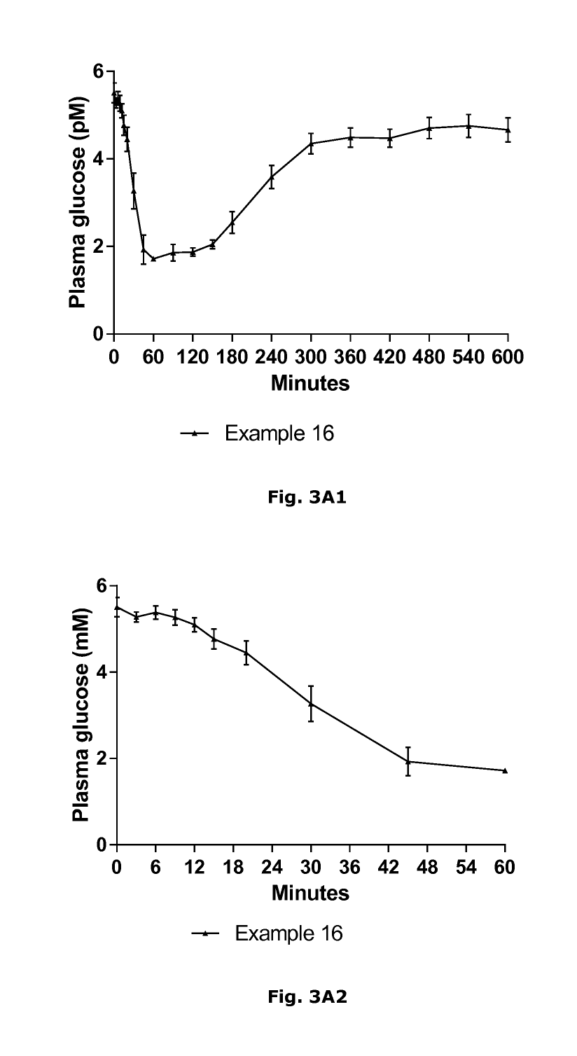

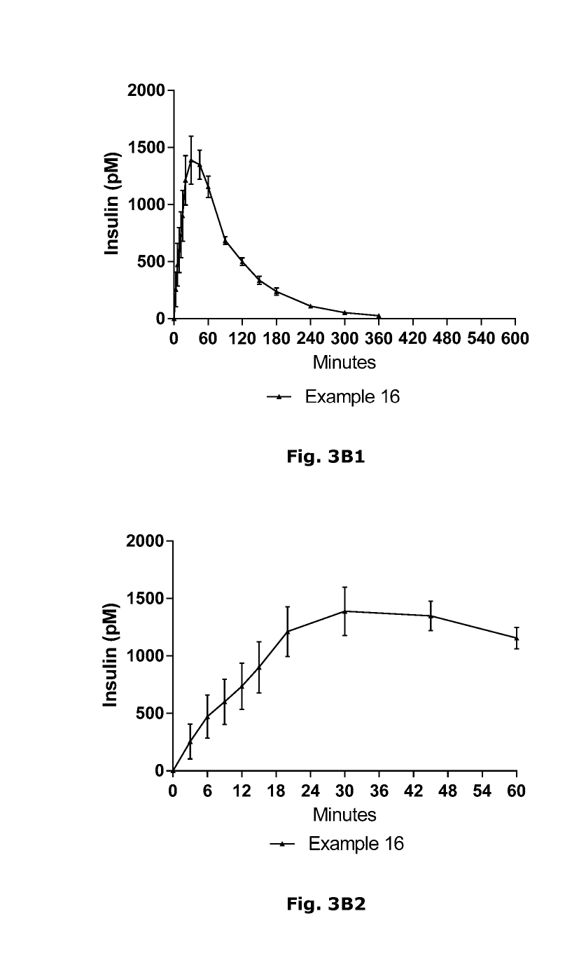

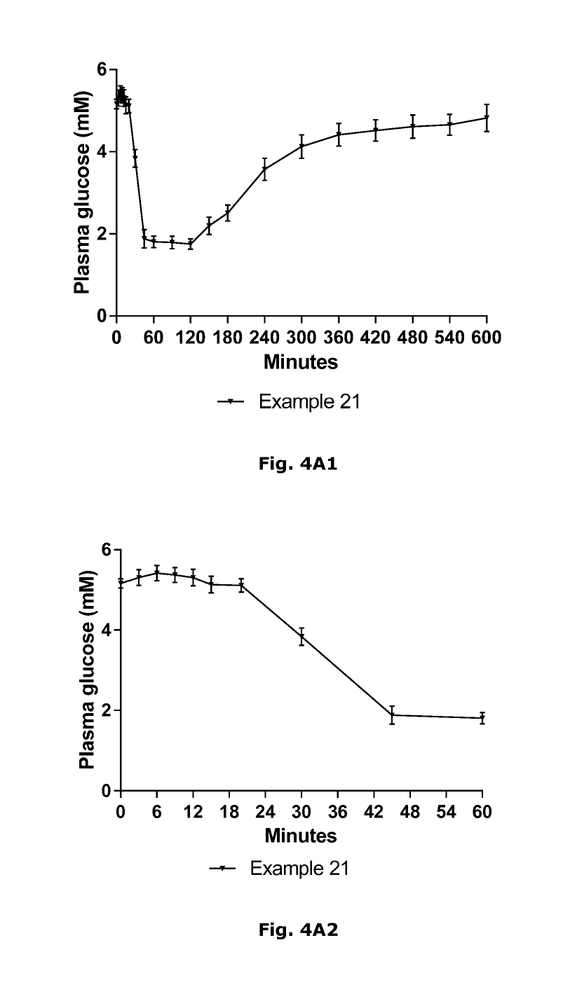

The results of these determinations are presented in the appended FIGS. 3A1, 3A2, 3B1, and 3B2, and in Table 7, below.

FIGS. 3A1, 3A2, 3B1, and 3B2 shows the PD (pharmacodynamic) and the PK (pharmacokinetic) profiles of the insulin derivative of Example 16, i.e. A21G, B3E, B28D, B29K(N(eps)tetradecanedioyl-4×gGlu), desB30 human insulin, formulated as described above with 0 zinc per 6 insulin molecules, and the resulting changes in plasma glucose, and the insulin concentrations vs. time, respectively (pigs were dosed 1 nmol/kg).

TABLE 7
Pharmacokinetic parameters after sc. dosing of 1 nmol/kg
of the compound of Example [symbol] 16 to pigs

| | | | Cₘₐₓ/D | | | | | |
|---|---|---|---|---|---|---|---|---|
| | | | pM/ | AUC/D | | | | |
| Com- | | Tₘₐₓ^{a} | (nmol/ | pM*min/ | % | T½^{b} | MRT | F^{c} |
| pound | | min | kg) | (pmol/kg) | extrap | min | min | % |
| Exam- | Mean | 30 | 1507 | 150 | 1 | 45 | 92 | 85 |
| ple 16 | | | | | | | | |
| (n = 8) | SD | | 501 | 18 | | 7 | 24 | 10 |

^{a}Tₘₐₓ given as median
^{b}T_{1/2} given as harmonic mean ± pseudoSD
^{c}Bioavailability calculated based on iv. data (not shown).

It is concluded that the insulin derivative of Example 16, in a formulation without zinc, is associated with an attractive prandial profile with fast lowering of plasma glucose and with a short plasma Tₘₐₓ (30 minutes). Mean residence time (MRT) is only 92 minutes, making the analogue suitable for prandial use.

### Example 62

### Subcutaneous PK/PD Profile of the Insulin of Example 21 in LYD Pigs

Following the general procedure above, the following PK and PD profiles were obtained for the insulin derivative of Example 21.

### Formulations Used

The compound of Example 21, pH=7.35; 625.4 μM; 7 mM phosphate; 1.6% (w/vol) glycerol; 16 mM phenol; 16 mM m-cresol; 10 mM sodium chloride (0 Zn/hexamer); 1 nmol/kg.

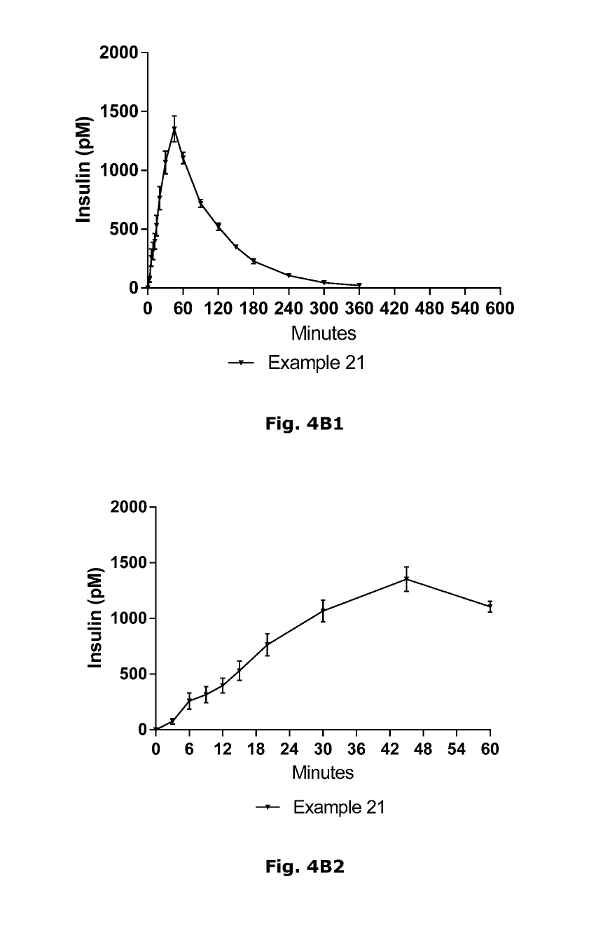

The results of these determinations are presented in the appended FIGS. 4A1, 4A2, 4B1, and 4B2, and in Table 8, below.

FIGS. 4A1, 4A2, 4B1, and 4B2 shows the PD (pharmacodynamic) and the PK (pharmacokinetic) profiles of the insulin derivative of Example 21, i.e. A21A, B3E, B27E, B28E, B29K(N(eps)tetradecanedioyl-4×gGlu), desB30 human insulin, formulated as described above with 0 zinc per 6 insulin molecules, and the resulting changes in plasma glucose, and the insulin concentrations vs. time, respectively (pigs were dosed 1 nmol/kg).

TABLE 8
Pharmacokinetic parameters after sc. dosing of
1 nmol/kg of the compound of Example 21 to pigs

| | | | Cₘₐₓ/D | AUC/D | | | | |
|---|---|---|---|---|---|---|---|---|
| Ex. | | Tₘₐₓ^{a} | pM/ | pM*min/ | | T½^{b} | MRT | F^{c} |
| No. | | (min.) | (nmol/kg) | (pmol/kg) | %ₑₓₜᵣₐₚ | (min.) | (min.) | % |
| 21 | Mean | 30 | 1362 | 139 | 1 | 45 | 97 | 71 |
| (n = 8) | SD | | 301 | 10 | | 9 | 15 | 5 |

^{a}Tₘₐₓ given as median
^{b}T_{1/2} given as harmonic mean ± pseudoSD
^{c}Bioavailability calculated based on iv. data (not shown).

It is concluded that the insulin derivative of Example 21, in a formulation without zinc, is associated with an attractive prandial profile with fast lowering of plasma glucose and with a short plasma Tₘₐₓ (30 minutes). Mean residence time (MRT) is only 97 minutes, making the analogue suitable for prandial use.

### Example 63

### Subcutaneous PK/PD Profile of the Prior Art Analogue 2 in LYD Pigs

Following the general procedure above, the following PK and PD profiles were obtained for the insulin Prior Art Analogue 2.

### Formulations Used

The compound of insulin Prior Art Analogue 2, pH=7.4; 610 μM; 1.6% (w/vol) glycerol; 30 mM phenol; (0 Zn/hexamer); 1 nmol/kg.

3 Zn formulation: The compound of insulin Prior Art Analogue 2, pH=7.4; 610 μM; 7 mM tris; 1.6% (w/vol) glycerol; 30 mM phenol; 300 μM zinc acetate (3 Zn/hexamer—or 3Zn/6 insulins); 1 nmol/kg.

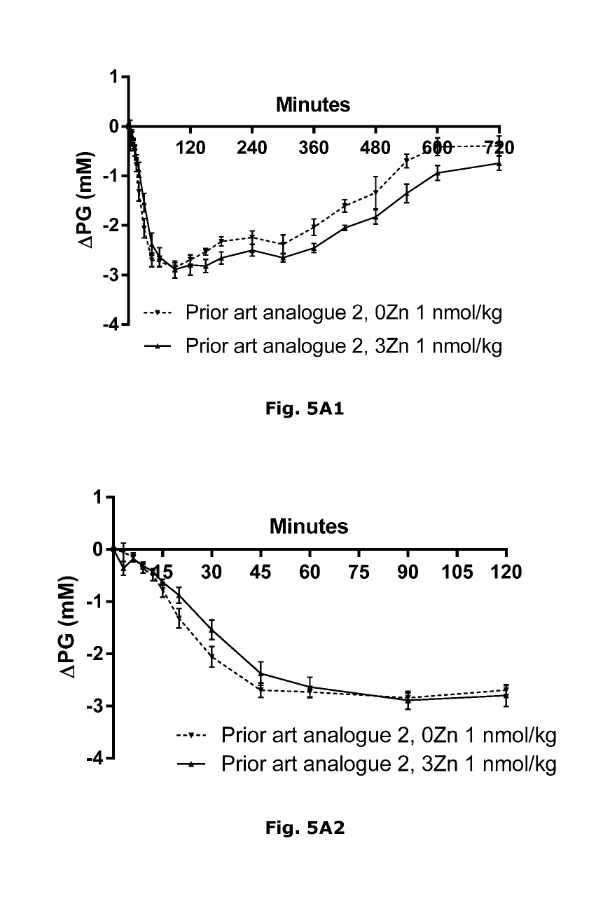

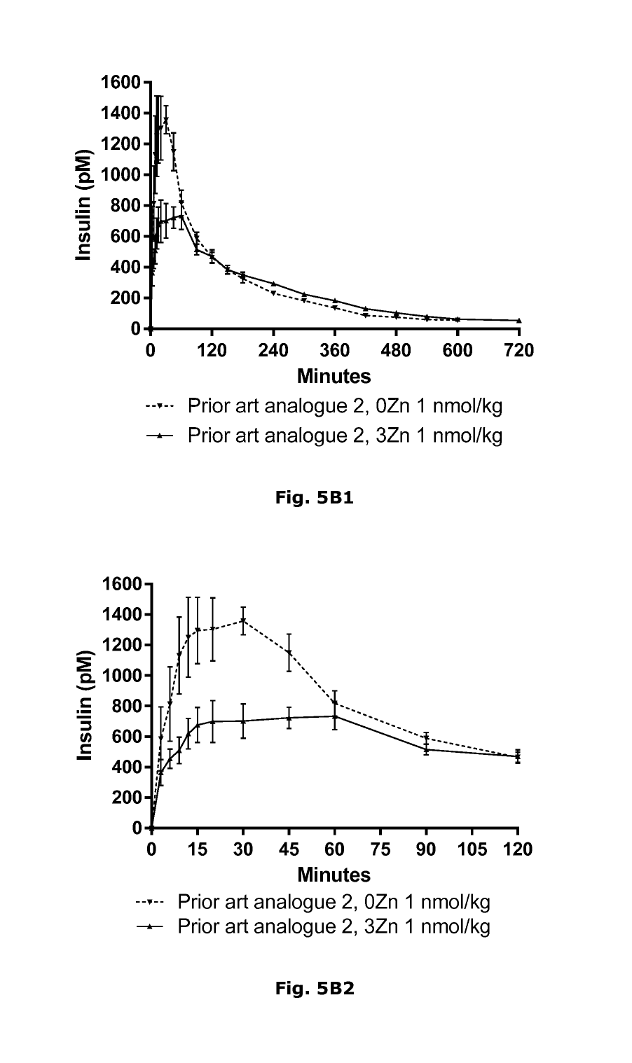

The results of these determinations are presented in the appended FIGS. 5A1, 5A2, 5B1, and 5B2, and in Table 9, below.

FIGS. 5A1, 5A2, 5B1, and 5B2 shows the PD (pharmacodynamic) and the PK (pharmacokinetic) profiles of the insulin derivative of Prior Art Analogue 2, i.e. B28D, B29K(N(eps)tetradecanedioyl-4×gGlu), desB30 human insulin, formulated as described above with 0 or 3 zinc per 6 insulin molecules, and the resulting changes in plasma glucose, and the insulin concentrations vs. time, respectively (pigs were dosed 1 nmol/kg).

TABLE 9
Pharmacokinetic parameters after sc. dosing of 1 nmol/kg
of the compound of Prior Art Analogue 2 to pigs

| | | | Cₘₐₓ/D | AUC/D | | | | |
|---|---|---|---|---|---|---|---|---|
| | | Tₘₐₓ^{b} | pM/ | pM*min/ | | T½^{c} | MRT | F^{d} |
| Cp.^{a} | | (min.) | (nmol/kg) | (pmol/kg) | %ₑₓₜᵣₐₚ | (min.) | (min.) | (%) |
| PA 2 | Mean | 30 | 1674 | 189 | 6 | 121 | 166 | 113 |
| (0 Zn) | | | | | | | | |
| (n = 8) | SD | | 578 | 21 | | 28 | 30 | 13 |
| PA 2 | Mean | 38 | 938 | 183 | 8 | 159 | 237 | 109 |
| (3 Zn) | | | | | | | | |
| (n = 8) | SD | | 264 | 21 | | 17 | 32 | 13 |

^{a}PA refers to Prior Art compound
^{b}Tₘₐₓ given as median
^{c}T_{1/2} given as harmonic mean ± pseudoSD
^{d}Bioavailability calculated based on iv. data (not shown).

It is concluded that the insulin derivative of the prior art, in a formulation without zinc, is associated with a profile with significant lowering of plasma glucose for at least 8 hours (280 minutes). Further, this analogue, formulated without zinc, is associated with both long T_{1/2} (half-life) and MRT (mean residence time), 121 and 166 minutes, respectively. These properties makes the analogue inappropriate for prandial use. Furthermore, in order to confer adequate chemical and physical stability in formulation, this analogue need to be formulated with zinc (as described above). Addition of 3 zinc ions per hexamer to the formulation further worsens the pharmacodynamic and pharmacokinetic properties. Plasma glucose is lowered for at least 10 hours, and the PK profile is associated with a peak-less maximal concentration and significant longer T_{1/2} and MRT (159 and 237 hours, respectively) compared with the profile of the 0 zinc formulation.

It is concluded that the insulin derivative of the prior art is inappropriate for prandial use.

### Example 64

### Subcutaneous PK/PD Profiles of Insulin Analogues of the Invention and of the Prior Art in Sprague Dawley Rats

The insulin derivatives of the invention may be tested by subcutaneous administration to rats, e.g. comparing with insulin aspart (NovoRapid) in the commercial formulation or comparing with similar B29K acylated insulin analogues of the prior art according to this protocol. The derivatives may be tested for pharmacokinetic and/or pharmacodynamic parameters.

The insulin derivatives of the prior art are only stable in formulation in presence of zinc ions, whereas the insulin derivatives of the present invention are stable in formulation without added zinc. In order to compare the profiles of the insulin derivatives of the invention to the profiles of the analogues of the prior art, the analogues of the invention are tested in this protocol using zinc-free formulations, and the analogues of the prior art are tested using 3 zinc ions per hexamer. This is to obtain the fastest PK profiles obtainable in clinically useful (i.e. chemically and physically stable) formulations.

### In Vivo Protocol

Male Sprague-Dawley rats, ˜400 grams, are used for these experiments. The rats are not fasted prior to testing. During the three hours study period, the rats have free access to water but not to food. Blood samples are drawn (sublingual vein; 200 μl into Microvette®200 EDTA tubes) and plasma collected from non-anesthetized animals at the time points 0 (before dosing) and 3, 7, 15, 30, 60, 120 and 180 minutes after dosing of the insulin derivative. The rats are dosed subcutaneously (25 nmol/kg; 600 μM formulation of insulin derivative) in the neck using a NovoPen Echo® mounted with a Softfine® 12 mm needle. Plasma concentrations of glucose and insulin derivatives are quantified using a BIOSEN analyser and immuno assays/LCMS analysis, respectively.

Results from testing analogues of the invention and of the prior art are given in Tables 10 and 11 and in the following figures:

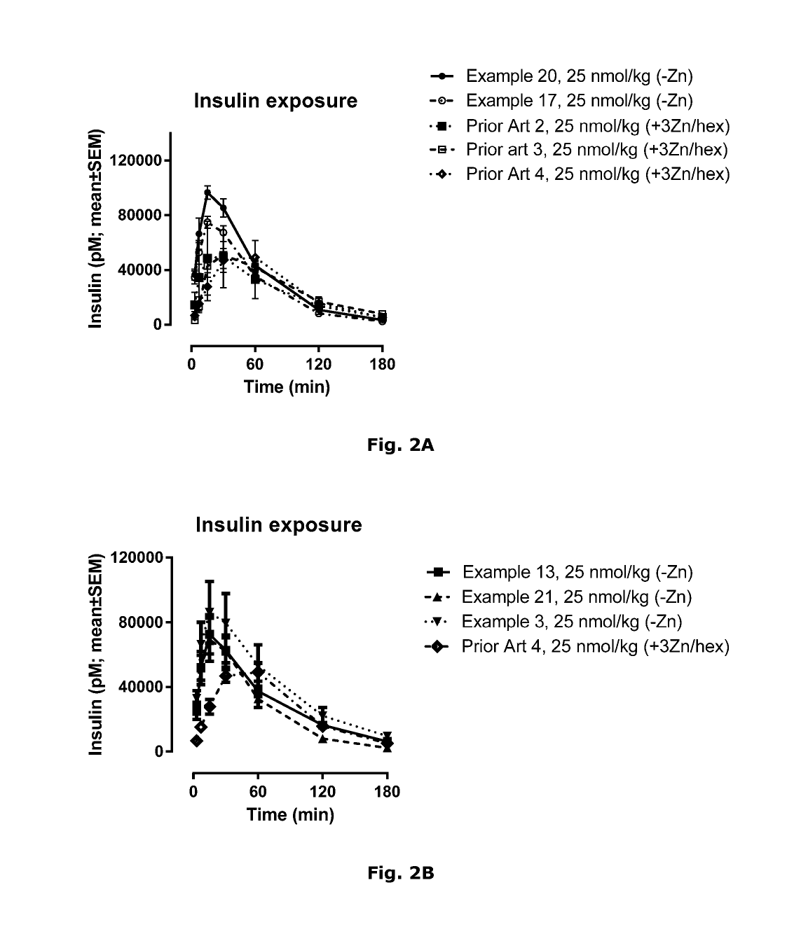

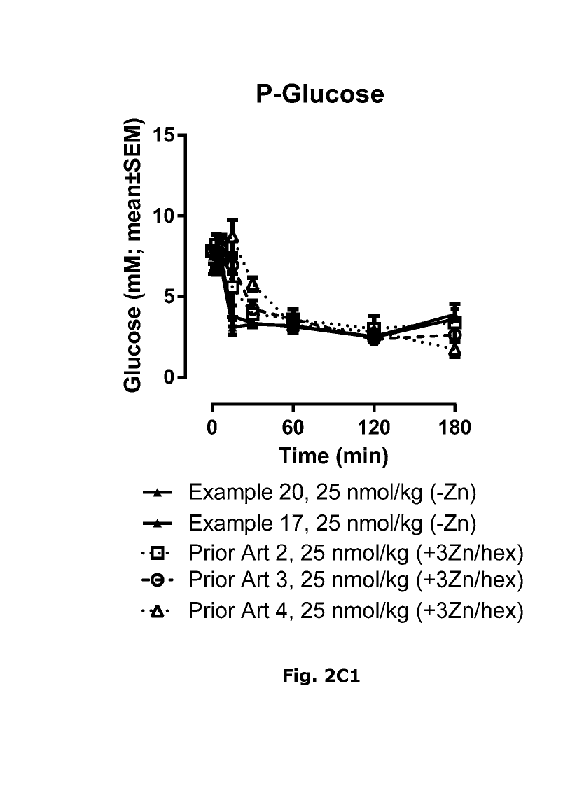

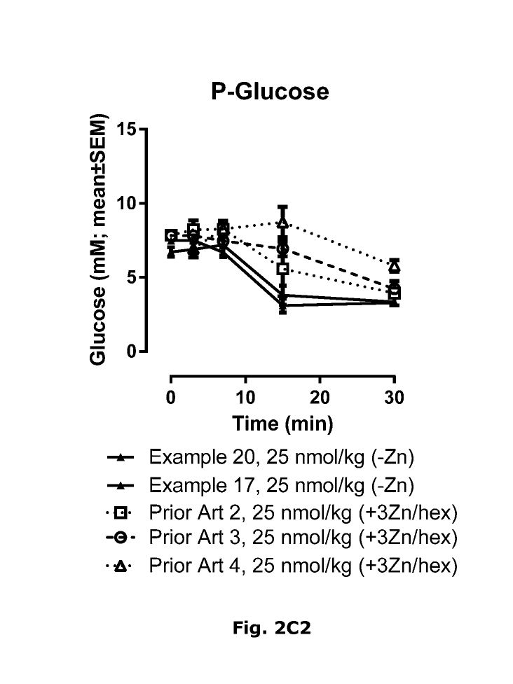

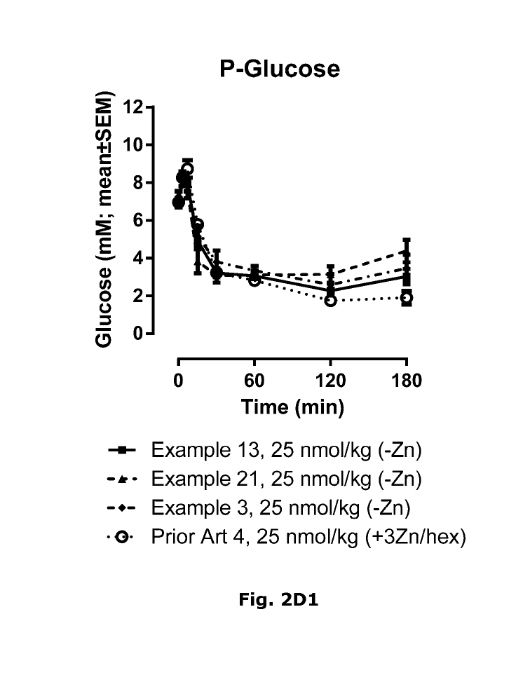

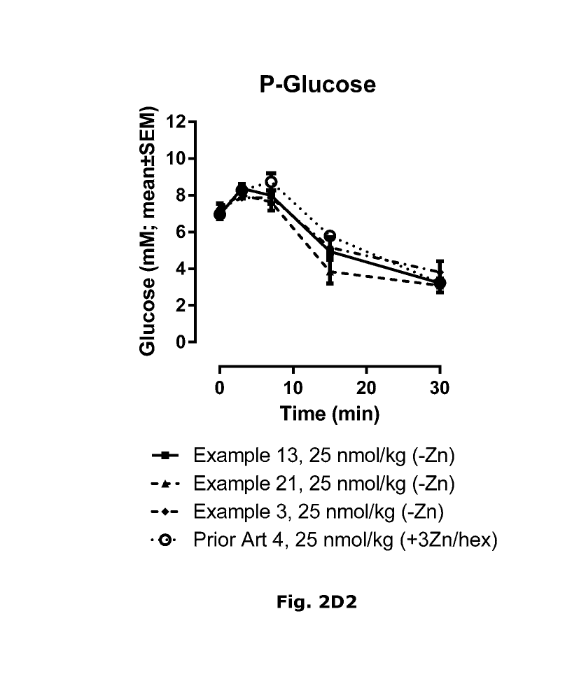

FIGS. 2A and 2B shows PK profiles of analogues of the invention (Examples 17 and 20, and Examples 3, 13 and 21, respectively) and of analogues of the prior art (Prior Art Analogues 2, 3 and 4 and Prior Art Analogue 4, respectively) following subcutaneous injection to Sprague Dawley rats. FIGS. 2C1 and 2C2 shows PD profiles of analogues of the invention (Examples 17 and 20) and of analogues of the prior art (Prior Art Analogues 2, 3 and 4) and FIGS. 2D1 and 2D2 shows PD profiles of analogues of the invention (Examples 3, 13 and 21) and of analogues of the prior art (Prior Art Analogue 4) following subcutaneous injection to Sprague Dawley rats.

TABLE 10
Selected PK parameters of C14 diacid acylated insulins
of the invention and of insulins of the prior art following
subcutaneous injection to Sprague Dawley rats

| | Zn in | | | | | | |
|---|---|---|---|---|---|---|---|
| Ex. | formu- | HSA | Tₘₐₓ | Cₘₐₓ | AUC15/ | MRT | T½ |
| No.^{a} | lation* | binder^{b} | (min) | (pmol) | AUC60** | (min) | (min) |
| 20 | −Zn | C14 | 15 | 98880 | 0.22 | 54 | 32 |
| | | | | (10092) | (0.03) | (3.2) | (1.8) |
| 17 | −Zn | C14 | 15 | 75000 | 0.22 | 52 | 30 |
| | | | | (8683) | (0.04) | (6.0) | (2.5) |
| 13 | −Zn | C14 | 15 | 73800 | 0.22 | 74 | 47 |
| | | | | (25521) | (0.03) | (6.5) | (2.2) |
| 21 | −Zn | C14 | 15 | 82400 | 0.23 | 53 | 31 |
| | | | | (13431) | (0.02) | (3.9) | (2.4) |
| 3 | −Zn | C14 | 15 | 87960 | 0.22 | 77 | 49 |
| | | | | (45086) | (0.03) | (6.0) | (1.7) |
| PA 2 | +3Zn/ | C14 | 30 | 51120 | 0.18 | 77 | 45 |
| | hex | | | (25218) | (0.03) | (7.2) | (4.9) |
| PA 3 | +3Zn/ | C14 | 30 | 52700 | 0.11 | 90 | 50 |
| | hex | | | (18294) | (0.03) | (17) | (10) |
| PA 4 | +3Zn/ | C14 | 45 | 55750 | 0.10 | 79 | 38 |
| | hex | | | (8586) | (0.01) | (5.7) | (4.1) |
| PA 7 | +3Zn/ | C16 | 120 | 53667 | 0.03 | 304 | 200 |
| | hex | | | (11896) | (0.01) | (35) | (9) |

SD values are given in parentheses
^{a}PA refers to Prior Art compound
^{b}C14 means side chain based on 1,14-tetradecanedioic acid and C16 means side chain based on 1,16-hexadecanedioic acid
*−Zn means no added zinc ions; +3Zn/hex means 3 added zinc ions per hehamer (6 insulin molecules)
**AUC15/AUC60 is the area under the curve (plasma exposure vs. time) for the first 15 minutes divided by the area under the curve for the first 60 minutes

### Conclusion, C14 Diacid Acylated Insulins:

It is concluded that the C14 diacid acylated analogues of the invention (in formulations without zinc) are absorbed more rapidly than the the analogues of the prior art (in formulations with 3 zinc ions per hexamer) as seen for the Tₘₐₓ data. Tₘₐₓ of the prior art analogues are of from 30 to 120 minutes whereas the insulins of the invention have Tₘₐₓ around 15 minutes. The ratio AUC15/AUC60 is a measure of the fraction absorbed during the first 15 minutes in relation to the fraction absorbed after 1 hour. Thus, the higher the ratio the more insulin is absorbed during the first 15 minutes. It is seen that the insulins of the invention are associated with a higher ratio than similar analogues of the prior art and are thus more rapidly absorbed.

Consequently, the analogues of the invention are better suited for prandial administration than insulins of the prior art.

TABLE 11
Selected PK parameters of C16 diacid acylated insulins
of the invention and of insulins of the prior art following
subcutaneous injection to Sprague Dawley rats

| | Zn in | | | | | | |
|---|---|---|---|---|---|---|---|
| Ex. | formu- | HSA | Tₘₐₓ | Cₘₐₓ | AUC15/ | MRT | T½ |
| No.^{a} | lation* | binder^{b} | (min) | (pmol) | AUC60** | (min) | (min) |
| 51 | −Zn | C16 | 30 | 102820 | 0.16 | 103 | 60 |
| | | | | (26744) | (0.03) | (18) | |
| 52 | −Zn | C16 | 30 | 138000 | 0.19 | 139 | 90 |
| | | | | (28994) | (0.02) | (21) | (13) |
| PA 1 | +3Zn/ | C16 | 60 | 63950 | 0.07 | — | — |
| | hex | | | (15706) | (0.01) | | |
| PA 7 | +3Zn/ | C16 | 120 | 53667 | 0.03 | 304 | 200 |
| | hex | | | (11896) | (0.01) | (35) | (9) |

SD values are given in parentheses
^{a}PA refers to Prior Art compound
^{b}C16 means side chain based on 1,16-hexadecanedioic acid
*−Zn means no added zinc ions; +3Zn/hex means 3 added zinc ions per hehamer (6 insulin molecules)
**AUC15/AUC60 is the area under the curve (plasma exposure vs. time) for the first 15 minutes divided by the area under the curve for the first 60 minutes

### Conclusion, C16 Diacid Acylated Insulins:

It is concluded that the C16 diacid acylated analogues of the invention (in formulations without zinc) are absorbed more rapidly than the the analogues of the prior art (in formulations with 3 zinc ions per hexamer) as seen for the Tₘₐₓ data. Tₘₐₓ of the prior art analogues are of from 60 to 120 minutes whereas the insulins of the invention have Tₘₐₓ around 30 minutes. The ratio AUC15/AUC60 is a measure of the fraction absorbed during the first 15 minutes in relation to the fraction absorbed after 1 hour. Thus, the higher the ratio the more insulin is absorbed during the first 15 minutes. It is seen that the insulins of the invention are associated with a higher ratio than similar analogues of the prior art and are thus more rapidly absorbed.

Consequently, the analogues of the invention are better suited for prandial administration than insulins of the prior art.

1. An acylated analogue of human insulin, which analogue is [B3aar¹, desB30] relative to human insulin; wherein
aar¹ represents Glu (E), Gln (Q), Asp (D), Ser (S) or Thr (T); and
one or two of the amino acid residues located in positions B26, B27 and/or B28 are substituted for Glu (E) and/or Asp (D);
which analogue may additionally comprise an A8aar² substitution, and/or an A14Glu (E) substitution, and/or an A21aar³ substitution; wherein
aar² represents His (H) or Arg (R); and
aar³ represents Gly (G) or Ala (A);
which insulin analogue is derivatized by acylation of the epsilon amino group of the naturally occurring lysine residue at the B29 position with a group of Formula II
[Acyl]-[Linker]-
wherein the Linker group is an amino acid chain composed of from 1 to 10 amino acid residues selected from gGlu and/or OEG; wherein
gGlu represents a gamma glutamic acid residue;
OEG represents a residue of 8-amino-3,6-dioxaoctanoic acid (i.e. a group of the formula —NH—(CH₂)₂—O—(CH₂)₂—O—CH₂—CO—);
which amino acid residues may be present in any order; and
which amino acid chain comprises at least one gGlu residue; and
wherein the Acyl group is a residue of an α,ω-di-carboxylic acid selected from 1,14-tetradecanedioic acid; 1,15-pentadecanedioic acid; and 1,16-hexadecanedioic acid.

2. (canceled)

3. The acylated insulin analogue according to claim 1, which analogue is [B3aar¹, B26aar⁴, desB30] relative to human insulin; wherein
aar¹ represents Glu (E), Gln (Q), Asp (D), Ser (S) or Thr (T); and
aar⁴ represents Glu (E) and/or Asp (D).

4. (canceled)

5. (canceled)

6. The acylated insulin analogue according to claim 1, which analogue is [B3aar¹, B26aar⁴, B28aar⁴, desB30] relative to human insulin; wherein
aar¹ represents Glu (E), Gln (Q), Asp (D), Ser (S) or Thr (T); and
aar⁴ independently of each other represent Glu (E) and/or Asp (D).

7. The acylated insulin analogue according to claim 1, which analogue is [B3aar¹, B27aar⁴, B28aar⁴, desB30] relative to human insulin; wherein
aar¹ represents Glu (E), Gln (Q), Asp (D), Ser (S) or Thr (T); and
aar⁴ independently of each other represent Glu (E) and/or Asp (D).

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. The acylated insulin analogue according to claim 1, which analogue is
[A8H, A21A, B3E, B28D, desB30];
[A8H, A21G, B3E, B27E, B28E, desB30];
[A8H, A21G, B3E, B28D, desB30];
[A8H, B3E, B27E, B28E, desB30];
[A8H, B3E, B28D, desB30];
[A14E, A21A, B3Q, B28D, desB30;
[A14E, B3Q, B28D, desB30];
[A21A, B3E, B26E, desB30];
[A21A, B3E, B26E, B28E, desB30];
[A21A, B3E, B27E, B28E, desB30];
[A21A, B3E, B28D, desB30];
[A21A, B3E, B28E, desB30];
[A21A, B3Q, B28D, desB30];
[A21G, B3E, B26E, desB30];
[A21G, B3E, B26E, B28E, desB30];
[A21G, B3E, B27E, desB30];
[A21G, B3E, B27E, B28D, desB30];
[A21G, B3E, B27E, B28E, desB30];
[A21G, B3E, B28D, desB30];
[A21G, B3E, B28E, desB30];
[B3E, B26E, desB30];
[B3E, B26E, B28E, desB30];
[B3E, B27E, B28E, desB30];
[B3E, B28E, desB30];
[B3E, B28D, desB30];
[B3Q, B26E, desB30];
[B3Q, B28E, desB30]; or
[B3Q, B28D, desB30];
relative to human insulin.

20. The acylated insulin analogue according to claim 1, wherein, in the group of Formula II
[Acyl]-[Linker]-
the Linker group is an amino acid chain composed of from 1 to 10 amino acid residues selected from gGlu and/or OEG; which amino acid residues may be present in any order; and which amino acid chain comprises at least one gGlu residue.

21. The acylated insulin analogue according to claim 1, wherein, in the group of Formula II
[Acyl]-[Linker]-
the Acyl group is a residue of an α,ω-di-carboxylic acid selected from 1,14-tetradecanedioic acid; 1,15-pentadecanedioic acid; and 1,16-hexadecanedioic acid.

22. The acylated insulin analogue according to claim 1, wherein the group of Formula II is
tetradecanedioyl-gGlu-2×OEG;
tetradecanedioyl-4×gGlu;
hexadecanedioyl-gGlu-2×OEG; or
hexadecanedioyl-4×gGlu.

23. The acylated insulin analogue according to claim 1, which is
B3E, B26E, B29K(N(eps)tetradecanedioyl-4×gGlu), desB30 human insulin;
B3E, B26E, B29K(N(eps)tetradecanedioyl-gGlu-2×OEG), desB30 human insulin;
B3E, B26E, B28E, B29K(N(eps)tetradecanedioyl-4×gGlu), desB30 human insulin;
B3E, B26E, B28E, B29K(N(eps)tetradecanedioyl-gGlu-2×OEG), desB30 human insulin;
B3E, B27E, B28E, B29K(N(eps)tetradecanedioyl-4×gGlu), desB30 human insulin;
B3E, B27E, B28E, B29K(N(eps)tetradecanedioyl-gGlu-2×OEG), desB30 human insulin;
B3E, B28D, B29K(N(eps)tetradecanedioyl-4×gGlu), desB30 human insulin;
B3E, B28D, B29K(N(eps)tetradecanedioyl-gGlu-2×OEG), desB30 human insulin;
B3E, B28E, B29K(N(eps)tetradecanedioyl-4×gGlu), desB30 human insulin;
B3E, B28E, B29K(N(eps)tetradecanedioyl-gGlu-2×OEG), desB30 human insulin;
B3Q, B28D, B29K(N(eps)tetradecanedioyl-4×gGlu), desB30 human insulin;
B3Q, B28D, B29K(N(eps)tetradecanedioyl-gGlu-2×OEG), desB30 human insulin;
A8H, B3E, B27E, B28E, B29K(N(eps)tetradecanedioyl-gGlu-2×OEG), desB30 human insulin;
A8H, B3E, B28D, B29K(N(eps)tetradecanedioyl-4×gGlu), desB30 human insulin;
A8H, B3E, B28D, B29K(N(eps)tetradecanedioyl-gGlu-2×OEG), desB30 human insulin;
A8H, A21A, B3E, B27E, B28E, B29K(N(eps)tetradecanedioyl-gGlu-2×OEG), desB30 human insulin;
A8H, A21A, B3E, B28D, B29K(N(eps)tetradecanedioyl-gGlu-2×OEG), desB30 human insulin;
A8H, A21G, B3E, B27E, B28E, B29K(N(eps)tetradecanedioyl-gGlu-2×OEG), desB30 human insulin;
A8H, A21G, B3E, B28D, B29K(N(eps)tetradecanedioyl-gGlu-2×OEG), desB30 human insulin;
A14E, A21A, B3Q, B28D, B29K(N(eps)tetradecanedioyl-4×gGlu), desB30 human insulin;
A14E, B3Q, B28D, B29K(N(eps)tetradecanedioyl-4×gGlu), desB30 human insulin;
A21A, B3E, B26E, B29K(N(eps)tetradecanedioyl-gGlu-2×OEG), desB30 human insulin;
A21A, B3E, B26E, B29K(N(eps)tetradecanedioyl-4×gGlu), desB30 human insulin;
A21A, B3E, B26E, B28E, B29K(N(eps)tetradecanedioyl-4×gGlu), desB30 human insulin;
A21A, B3E, B26E, B28E, B29K(N(eps)tetradecanedioyl-gGlu-2×OEG), desB30 human insulin;
A21A, B3E, B27E, B28E, B29K(N(eps)tetradecanedioyl-4×gGlu), desB30 human insulin;
A21A, B3E, B27E, B28E, B29K(N(eps)hexadecanedioyl-4×gGlu), desB30 human insulin;
A21A, B3E, B27E, B28E, B29K(N(eps)tetradecanedioyl-gGlu-2×OEG), desB30 human insulin;
A21A, B3E, B28D, B29K(N(eps)tetradecanedioyl-4×gGlu), desB30 human insulin;
A21A, B3E, B28D, B29K(N(eps)tetradecanedioyl-gGlu-2×OEG), desB30 human insulin;
A21A, B3E, B28E, B29K(N(eps)tetradecanedioyl-4×gGlu), desB30 human insulin;
A21A, B3E, B28E, B29K(N(eps)tetradecanedioyl-gGlu-2×OEG), desB30 human insulin;
A21A, B3Q, B28D, B29K(N(eps)tetradecanedioyl-gGlu-2×OEG), desB30 human insulin;
A21G, B3E, B26E, B29K(N(eps)tetradecanedioyl-4×gGluG), desB30 human insulin;
A21G, B3E, B26E, B29K(N(eps)tetradecanedioyl-gGlu-2×OEG), desB30 human insulin;
A21G, B3E, B26E, B28E, B29K(N(eps)tetradecanedioyl-4×gGlu), desB30 human insulin;
A21G, B3E, B26E, B28E, B29K(N(eps)tetradecanedioyl-gGlu-2×OEG), desB30 human insulin;
A21G, B3E, B27E, B29K(N(eps)tetradecanedioyl-4×gGlu), desB30 human insulin;
A21G, B3E, B27E, B28D, B29K(N(eps)tetradecanedioyl-gGlu-2×OEG), desB30 human insulin;
A21G, B3E, B27E, B28E, B29K(N(eps)tetradecanedioyl-gGlu-2×OEG), desB30 human insulin;
A21G, B3E, B28D, B29K(N(eps)tetradecanedioyl-4×gGlu), desB30 human insulin;
A21G, B3E, B28D, B29K(N(eps)tetradecanedioyl-gGlu-2×OEG), desB30 human insulin;
A21G, B3E, B28D, B29K(N(eps)hexadecanedioyl-4×gGlu), desB30 human insulin;
A21G, B3E, B28D, B29K(N(eps)hexadecanedioyl-gGlu-2×OEG), desB30 human insulin;
A21G, B3E, B28E, B29K(N(eps)tetradecanedioyl-4×gGlu), desB30 human insulin;
A21G, B3E, B28E, B29K(N(eps)tetradecanedioyl-gGlu-2×OEG), desB30 human insulin;
B3Q, B26E, B29K(N(eps)tetradecanedioyl-4×gGlu), desB30 human insulin;
A21A, B3Q, B28E, B29K(N(eps)tetradecanedioyl-4×gGlu), desB30 human insulin;
B3Q, B28E, B29K(N(eps)tetradecanedioyl-4×gGlu), desB30 human insulin;
A21A, B3Q, B26E, B29K(N(eps)tetradecanedioyl-4×gGlu), desB30 human insulin;
A21A, B3E, B28E, B29K(N(eps)hexadecanedioyl-4×gGlu), desB30 human insulin; or
A21A, B3E, B28E, B29K(N(eps)hexadecanedioyl-gGlu-2×OEG), desB30 human insulin.

24. A pharmaceutical composition comprising an insulin derivative according to claim 1, and one or more pharmaceutically acceptable carriers or diluents.

25. The pharmaceutical composition according to claim 24, formulated as a low-zinc composition, with no added zinc ions.

26. The pharmaceutical composition according claim 24, formulated as a low-zinc composition, comprising less than 0.2 Zn²⁺ ions per 6 insulin molecules.

27. (canceled)

28. The low-zinc pharmaceutical composition according to claim 25, comprising a nicotinic compound, and in particular nicotinamide.

29. (canceled)

30. A method of treatment, prevention or alleviation of a metabolic disease or disorder or condition of a living animal body, including a human, which method comprises the step of administering to such a living animal body in need thereof, a therapeutically effective amount of the acylated insulin analogue according to claim 1.
